(19) United States
(12) Patent Application Publication
(10) Pub. No.: US 2025/0115919 A1
(43) Pub. Date: Apr. 10, 2025

(54) COMPOSITIONS AND METHODS RELATED TO APTAMER-BASED COLORIMETRIC ASSAYS

(71) Applicant: North Carolina State University, Raleigh, NC (US)

(72) Inventors: Yi Xiao, Raleigh, NC (US); Juan Canoura, Raleigh, NC (US)

(21) Appl. No.: 18/884,798

(22) Filed: Sep. 13, 2024

Related U.S. Application Data
(60) Provisional application No. 63/582,330, filed on Sep. 13, 2023.

(51) Int. Cl. C12N 15/115; G01N 33/58; G01N 33/94

(52) U.S. Cl. CPC C12N 15/115; G01N 33/582; G01N 33/9486; C12N 2310/16

(57) ABSTRACT

The present disclosure provides compositions and methods related to opioid detection. In particular, the present disclosure provides aptamer-based colorimetric assays for the visualization and identification of specific opioids (natural, semi-synthetic, synthetic and the derivatives and analogs thereof) in a manner that is rapid, specific, and sensitive.

### CROSS REFERENCE TO RELATED APPLICATIONS

This application claims priority to and the benefit of U.S. Provisional Patent Application No. 63/582,330 filed Sep. 13, 2023, which is incorporated herein by reference in its entirety for all purposes.

### STATEMENT REGARDING FEDERALLY SPONSORED RESEARCH OR DEVELOPMENT

This invention was made with government support under grant number 2019-DU-BX-0024 awarded by the National Institute of Justice. The government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: 171,146 bytes; XML file named “NCSU_42149_202_SequenceListing” created on Sep. 13, 2024.

### FIELD

The present disclosure provides compositions and methods related to opioid detection. In particular, the present disclosure provides aptamer-based colorimetric assays for the visualization and identification of specific opioids (natural, semi-synthetic, synthetic and the derivatives and analogs thereof) in a manner that is rapid, specific, and sensitive.

### BACKGROUND

Opioids are a commonly used class of analgesics that can be categorized as natural, semi-synthetic, or fully synthetic. Natural opioids (i.e., opiates) such as morphine and codeine are derived from the poppy plant Papaver somniferum, whereas semi-synthetic opioids include compounds such as heroin, hydrocodone, oxycodone, hydromorphone, and oxymorphone that are produced by the chemical modification of these opiates. Synthetic opioids such as fentanyl, tramadol, and methadone are entirely manmade. Despite their structural differences, all opioids bind and activate μ-opioid receptors in the body, resulting in analgesia, sedation, and a reduction in respiratory rate. Although these drugs have great utility in medical settings, they also have a long history of misuse and abuse primarily due to their euphoric and addictive effects. Heroin is one of the most well-known and commonly abused illicit drugs in the world, and prescription opioids such as morphine, codeine, hydrocodone, oxycodone, hydromorphone, and oxymorphone are also often diverted and abused. Opioid addiction represents a major public health crisis, and in 2021, 75% of the approximately 106,000 drug overdose deaths in the United States were attributed to opioids.

The detection and identification of opioids in seized substances is important for investigating and curtailing their trafficking and distribution. Opioids are often prepared as powders, pharmaceutical tablets (e.g., oxycodone, hydrocodone), and counterfeit tablets laced with fentanyl, which account for 17.6% of seized opioids. Additionally, fentanyl was the fourth most commonly identified drug in seized substances in the United States in 2021, followed by heroin in fifth place, and oxycodone in eighth place. While methodologies such as mass spectrometry can detect opioids with high accuracy and precision, they are expensive and complex, have low throughput, and are primarily reserved for confirmatory analysis. More expedient means of screening opioids in drug samples include analysis by chemical testing. The Marquis Test, which is often used to identify morphine-like opioids, entails mixing a drug sample with formaldehyde and sulfuric acid, where the resulting reaction results in two opioid molecules being joined together by formaldehyde to form a more extensively conjugated, purple-colored product. Although this test is simple to perform and provides results relatively quickly, it is prone to false-positives and negatives in response to cutting agents, adulterants, and other drugs of abuse. For instance, the Marquis test yields similar colors as opioids with diverse substances such as acetylsalicylic acid, 3,4-methylenedioxymethamphetamine (MDMA), and lysergic acid diethylamide (LSD). When challenged with street drugs, which are notorious for their low purity, this test suffers from interference due to the mixing and/or masking of the color produced by the opioid with that arising from other substances that generate their own distinct colors, such as methamphetamine and fentanyl (orange) and methadone (pink).

An alternative method for opioid screening entails the use of portable Raman spectrometers, which can identify drugs such as opioids based on inelastic scattering of light resulting from vibrational transitions between ground and virtual excited states. This approach is powerful because each molecule has its own unique Raman spectrum, making it highly specific. However, although Raman spectrometers excel at identifying highly abundant drugs in relatively pure matrices, this method faces challenges in detecting heroin, which is typically present alongside endogenous compounds from the opium plant that cause fluorescence interference, as well as fentanyl, which can be present at only trace amounts. Surface-enhanced Raman spectroscopy can overcome some of these issues but entails extra costs for specialized test kits and involves a more complex testing procedure. Sensors based on aptamers have the potential to overcome current challenges associated with opioid detection.

### SUMMARY

Embodiments of the present disclosure include a single-stranded nucleic acid molecule capable of specifically binding heroin or morphine, or a derivative or analog thereof, comprising a nucleic acid sequence that is at least 50% identical to the following: GGATTCG(X)₁₆₋₁₈CTCGT; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; X₁₅ is A, T, C, or G; X₁₆ is A, T, C, or G; X₁₇ is A, T, C, or G; and X₁₈ is A, T, C, or G (SEQ ID NO: 1). In accordance with these embodiments, (X)₁₆₋₁₈ indicates that X₁₋₁₆ are present in the nucleic acid molecule, but X₁₇ and X₁₈ are optional.

In some embodiments, X₁ is G or C; X₂ is A or G; X₃ is T or C; X₄ is C; X₅ is G or C; X₆ is T or C; X₇ is G or A; X₈ is G or A; X₉ is A or G; X₁₀ is A or T; X₁₁ is C or G; X₁₂ is A or G; X₁₃ is G; X₁₄ is T or G; X₁₅ is G or A; X₁₆ is C or G; X₁₇ is G; and X₁₈ is G (SEQ ID NO: 2). In some embodiments, the nucleic acid molecule comprises a nucleic acid sequence that is at least 50% identical to SEQ ID NOs: 9 or 10. In some embodiments, the nucleic acid molecule comprises a K_{D} that is less than about 900 nM.

Embodiments of the present disclosure also include a single-stranded nucleic acid molecule capable of specifically binding heroin or morphine, or a derivative or analog thereof, comprising a nucleic acid sequence that is at least 50% identical to the following: GCGTA(X)₇TAG(X)₅CGTCGTTCAA; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; and X 12 is A, T, C, or G (SEQ ID NO: 3).

In some embodiments, X₁ is G or C; X₂ is G, C, or T; X₃ is T or A; X₄ is T, C, or G; X₅ is T, C, or G; X₆ is C, G, or A; X₇ is C or G; X₈ is C or T; X₉ is T or C; X₁₀ is G; X₁₁ is T or C; and X₁₂ is G or A (SEQ ID NO: 4). In some embodiments, the nucleic acid molecule comprises a nucleic acid sequence that is at least 50% identical to any one of SEQ ID NOs: 11-17. In some embodiments, the nucleic acid molecule comprises a K_{D} that is less than about 6000 nM.

Embodiments of the present disclosure also include a single-stranded nucleic acid molecule capable of specifically binding heroin or morphine, or a derivative or analog thereof, comprising a nucleic acid sequence that is at least 50% identical to the following: TAGC(X)₃₋₄GCGTTGTTCGA(X)₆₋₈AGTA; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; and X₁₂ is A, T, C, or G (SEQ ID NO: 5). In accordance with these embodiments, (X)₃₋₄ indicates that X₁₋₃ are present in the nucleic acid molecule, but X₄ is optional. Additionally, (X)₆₋₈ indicates that X₁₆ are present in the nucleic acid molecule, but X₇ and X₈ are optional.

In some embodiments, X₁ is C, G, or T; X₂ is C or G; X₃ is A, T, C, or G; X₄ is G, T, or A; X₅ is G, A, or C; X₆ is T, C, or G; X₇ is A, T, C, or G; X₈ is C, A, or T; X₉ is A, G, or T; X₁₀ is G, T, or C; X₁₁ is C or G; and X₁₂ is G or A (SEQ ID NO: 6). In some embodiments, the nucleic acid molecule comprises a nucleic acid sequence that is at least 50% identical to any one of SEQ ID NOs: 18-26. In some embodiments, the nucleic acid molecule comprises a K_{D} that is less than about 3500 nM.

Embodiments of the present disclosure also include a single-stranded nucleic acid molecule capable of specifically binding heroin or morphine, or a derivative or analog thereof, comprising a nucleic acid sequence that is at least 50% identical to the following: AGGGCACGTCT(X)₆₋₈AGGGTTTCGCG; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; and X₈ is A, T, C, or G (SEQ ID NO: 7). In accordance with these embodiments, (X)₆₋₈ indicates that X₁₆ are present in the nucleic acid molecule, but X₇ and X₈ are optional.

In some embodiments, X₁ is C or G; X₂ is G or T; X₃ is G; X₄ is C or T; X₅ is A or G; X₆ is T or C; X₇ is C; and X₈ is G (SEQ ID NO: 8). In some embodiments, the nucleic acid molecule comprises a nucleic acid sequence that is at least 50% identical to SEQ ID NOs: 27 or 28. In some embodiments, the nucleic acid molecule comprises a K_{D} that is less than about 5000 nM.

In some embodiments, a single-stranded nucleic acid molecule capable of specifically binding heroin or morphine, or a derivative or analog thereof, comprising a nucleic acid sequence that is at least 50% identical to any one of SEQ ID NOs: 29-33.

Embodiments of the present disclosure also include a single-stranded nucleic acid molecule capable of specifically binding oxycodone or oxymorphone, or a derivative or analog thereof, comprising a nucleic acid sequence that is at least 50% identical to the following: (X)₁₂₋₁₃TCTCAGCGAGTTCG(X)₃₋₄; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; X₁₅ is A, T, C, or G; X₁₆ is A, T, C, or G; and X₁₇ is A, T, C, or G (SEQ ID NO: 34). In accordance with these embodiments, (X)₁₂₋₁₃ indicates that X₁₋₁₂ are present in the nucleic acid molecule, but X₁₃ is optional. Additionally, (X)₃₋₄ indicates that X₁₋₃ are present in the nucleic acid molecule, but X₄ is optional.

In some embodiments, X₁ is A, T, or G; X₂ is A, C, or G; X₃ is G, C, or A; X₄ is T or G; X₅ is C or T; X₆ is A; X₇ is G; X₈ is G, C, or T; X₉ is C or T; X₁₀ is C; X₁₁ is T or A; X₁₂ is G or T; X₁₃ is C or G; X₁₄ is A, C, or T; X₁₅ is C, G, or T; X₁₆ is A or G; and X₁₇ is T or A (SEQ ID NO: 35). In some embodiments, the nucleic acid molecule comprises a nucleic acid sequence that is at least 50% identical to any one of SEQ ID NOs: 46-51. In some embodiments, the nucleic acid molecule comprises a K_{D} that is less than about 2500 nM.

Embodiments of the present disclosure also include a single-stranded nucleic acid molecule capable of specifically binding oxycodone or oxymorphone, or a derivative or analog thereof, comprising a nucleic acid sequence that is at least 50% identical to the following: GGCT(X)₁₁GTAGGGGT(X)₁CACGCT; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; and X₁₂ is A, T, C, or G (SEQ ID NO: 36).

In some embodiments, X₁ is T; X₂ is A or G; X₃ is G or C; X₄ is C or A; X₅ is A or T; X₆ is A or T; X₇ is A or G; X₈ is G or A; X₉ is C or G; X₁₀ is T or C; X₁₁ is A; and X₁₂ is C or A (SEQ ID NO: 37). In some embodiments, the nucleic acid molecule comprises a nucleic acid sequence that is at least 50% identical to SEQ ID NOs: 52 or 53. In some embodiments, the nucleic acid molecule comprises a K_{D} that is less than about 1000 nM.

Embodiments of the present disclosure also include a single-stranded nucleic acid molecule capable of specifically binding oxycodone or oxymorphone, or a derivative or analog thereof, comprising a nucleic acid sequence that is at least 50% identical to the following: (X)₂₋₆GGGAGT(X)₂₋₃GTTTGTGTGGGG(X)₂₋₆; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; and X₁₅ is A, T, C, or G (SEQ ID NO: 38). In accordance with these embodiments, (X)₂₋₃ indicates that X₁₋₂ are present in the nucleic acid molecule, but X₃ is optional. Additionally, (X)₂₋₆ indicates that X₁₋₂ are present in the nucleic acid molecule, but X₃, X₄, X₅, and X₆ are optional.

In some embodiments, X₁ is G; X₂ is A or G; X₃ is G; X₄ is T; X₅ is G; X₆ is C; X₇ is G, A, or T; X₈ is T or A; X₉ is T; X₁₀ is T, A, or G; X₁₁ is C or G; X₁₂ is C or T; X₁₃ is C; X₁₄ is G or C; and X₁₅ is A (SEQ ID NO: 39). In some embodiments, the nucleic acid molecule comprises a nucleic acid sequence that is at least 50% identical to any one of SEQ ID NOs: 54-56. In some embodiments, the nucleic acid molecule comprises a K_{D} that is less than about 2000 mM.

Embodiments of the present disclosure also include a single-stranded nucleic acid molecule capable of specifically binding oxycodone or oxymorphone, or a derivative or analog thereof, comprising a nucleic acid sequence that is at least 50% identical to the following: (X)₁₋₂CGTAGGGG(X)₁ACACGCT(X)₄₋₆GGGCTG(X)₁₋₂; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; and X₁₁ is A, T, C, or G (SEQ ID NO: 40). In accordance with these embodiments, (X)₁₋₂ indicates that X₁ is present in the nucleic acid molecule, but X₂ is optional. Additionally, (X)₄₋₆ indicates that X₄ is present in the nucleic acid molecule, but X₅ and X₆ are optional.

In some embodiments, X₁ is C or A; X₂ is T or G; X₃ is T, C, or A; X₄ is C or T; X₅ is T, A, or G; X₆ is A or C; X₇ is T, C, or A; X₈ is A or G; X₉ is G; X₁₀ is G or A; and X₁₁ is T or G (SEQ ID NO: 41). In some embodiments, the nucleic acid molecule comprises a nucleic acid sequence that is at least 50% identical to any one of SEQ ID NOs: 57-60. In some embodiments, the nucleic acid molecule comprises a K_{D} that is less than about 2500 nM.

Embodiments of the present disclosure also include a single-stranded nucleic acid molecule capable of specifically binding oxycodone or oxymorphone, or a derivative or analog thereof, comprising a nucleic acid sequence that is at least 50% identical to the following: ATGGGAT(X)₁₋₄ATGTGGTGT; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; and X₁₄ is A, T, C, or G (SEQ ID NO: 42).

In some embodiments, X₁ is A or G; X₂ is C or G; X₃ is G, A, or C; X₄ is A or G; X₅ is A, C, or G; X₆ is C, T, or A; X₇ is T, A, or G; X₈ is C or T; X₉ is G or C; X₁₀ is T or C; X₁₁ is T or G; X₁₂ is T, A, or G; X₁₃ is G; and X₁₄ is G or C (SEQ ID NO: 43). In some embodiments, the nucleic acid molecule comprises a nucleic acid sequence that is at least 50% identical to any one of SEQ ID NOs: 61-63. In some embodiments, the nucleic acid molecule comprises a K_{D} that is less than about 3500 nM.

Embodiments of the present disclosure also include a single-stranded nucleic acid molecule capable of specifically binding oxycodone or oxymorphone, or a derivative or analog thereof, comprising a nucleic acid sequence that is at least 50% identical to the following: (X)₃₋₄ATGTGGTGT(X)₇₋₈ATGGGAT(X)₃; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; and X₁₅ is A, T, C, or G (SEQ ID NO: 44). In accordance with these embodiments, (X)₃₋₄ indicates that X₃ is present in the nucleic acid molecule, but X₄ is optional. Additionally, (X)₇₋₈ indicates that X₇ is present in the nucleic acid molecule, but X₈ is optional.

In some embodiments, X₁ is G; X₂ is C or G; X₃ is G; X₄ is C; X₅ is C; X₆ is G or A; X₇ is G; X₈ is T; X₉ is C; X₁₀ is A; X₁₁ is G or T; X₁₂ is G; X₁₃ is G or C; X₁₄ is A; and X₁₅ is A (SEQ ID NO: 45). In some embodiments, the nucleic acid molecule comprises a nucleic acid sequence that is at least 50% identical to SEQ ID NOs: 64 or 65. In some embodiments, the nucleic acid molecule comprises a K_{D} that is less than about 4000 nM.

Embodiments of the present disclosure also include a single-stranded nucleic acid molecule capable of specifically binding oxycodone or oxymorphone, or a derivative or analog thereof, comprising a nucleic acid sequence that is at least 50% identical to any one of SEQ ID NOs: 66-70.

In some embodiments, the nucleic acid molecule comprises a detection moiety. In some embodiments, the nucleic acid molecule is in solution or attached to a substrate. Embodiments of the present disclosure also include a vector comprising any of the nucleic acid sequences of the aptamers described herein.

Embodiments of the present disclosure also include a method of detecting an opioid, or a derivative or analog thereof. In accordance with these embodiments, the methods include combining any of the nucleic acid molecules of the aptamers described herein to a fluorescent moiety with a quencher-labeled nucleic acid molecule that is at least partially complementary to the nucleic acid molecules of the aptamers described herein to form a quenched composition; and exposing the quenched composition to a sample comprising or suspected of comprising an opioid, or a derivative or analog thereof. In some embodiments, the presence of the opioid, or a derivative or analog thereof, in the sample displaces the quencher-labeled nucleic acid molecule, thereby producing a fluorescent signal proportional to the concentration of the opioid, or a derivative or analog thereof, in the sample.

Embodiments of the present disclosure also include a method of detecting opioid, or a derivative or analog thereof. In accordance with these embodiments, the methods include combining any of the nucleic acid molecules of the aptamers described herein with a reporter compound that binds to the nucleic acid molecules of the aptamers described herein to form a complexed composition; and exposing the complexed composition to a sample comprising or suspected of comprising an opioid, or a derivative or analog thereof. In some embodiments, the presence of the opioid, or a derivative or analog thereof, in the sample displaces the reporter compound, thereby allowing the reporter compound to form detectable aggregates proportional to the concentration of the opioid, or a derivative or analog thereof, in the sample.

Embodiments of the present disclosure also include a method of detecting opioid, or a derivative or analog thereof. In accordance with these embodiments, the methods include immobilizing any of the nucleic acid molecules of the aptamers described herein to an electrically conductive substrate, wherein the nucleic acid molecules of the aptamers described herein comprising a redox tag, to form a detection sensor; and exposing the detection sensor to a sample comprising or suspected of comprising an opioid, or a derivative or analog thereof. In some embodiments, the presence of the opioid, or a derivative or analog thereof, in the sample binds the nucleic acid molecules of the aptamers described herein, thereby producing an electrochemical signal proportional to the concentration of the opioid, or a derivative or analog thereof, in the sample.

In some embodiments, the sample is a biological sample from a human subject. In some embodiments, the biological sample is a saliva sample, a urine sample, a blood sample, a serum sample, a plasma sample, a fecal sample, a CSF sample, or a tissue sample.

### BRIEF DESCRIPTION OF THE DRAWINGS

FIGS. 1A-1C. SELEX targets and selection strategy. (A) Structure of the opioid targets. All opioids share the 4,5-epoxymorphinan core structure and vary at the indicated R groups and bond order between C7 and C8. (B) Library design. (C) Working principle of library-immobilized SELEX.

FIGS. 2A-2F. Isolation of opioid binding aptamers using library-immobilized SELEX. (A) Parallel and serial selection strategy for generating aptamers binding to morphine and heroin. (B) The percentage of pool eluted each round by target per micromolar concentration of heroin or morphine. (C) Cross-reactivity of the round S4 pool against heroin, morphine and interferents. Cross-reactivity is calculated relative to heroin. (D) Toggle selection strategy for generating aptamers binding to oxycodone and oxymorphone. (E) The percentage of pool eluted each round by target per micromolar concentration of oxycodone and oxymorphone. (F) Cross-reactivity of the round T6 pool against oxycodone, oxymorphone and interferents. Cross-reactivity is calculated relative to oxycodone.

FIGS. 3A-3H. High-throughput sequencing (HTS) analysis of enriched aptamer pools. Percent of unique sequences from (A) rounds S1-4 and (B) rounds T1, T2, T4 and T6. Four categories of aptamers were defined based on their growth pattern during SELEX: (C, D) exponential or linear growth (Category 1), (E) a gradually reducing growth rate (Category 2), (F) peaked (Category 3), and (G, H) declining between rounds (Category 4).

FIGS. 4A-4B. Binding affinity characterization of isolated aptamers. Isothermal titration calorimetry (ITC)-derived binding affinity of the 25 aptamers from category 1, 2, and 3 isolated from (A) HM and (B) OM SELEX.

FIG. 5. Scheme for the T5 Exo/Exo I fluorescence assay.

FIGS. 6A-6D. Using exonuclease digestion fluorescence assays to determine the binding profile of opioid aptamers. (A, B) Binding affinity and (C, D) specificity of HM and OM aptamers for various opioids and interferents as determined using the T5 Exo/Exo I fluorescence assay. Data are presented as heat maps, with each square representing one aptamer and color intensity corresponding to resistance value. Increasing color intensity indicates higher resistance values, and hence tighter aptamer-ligand binding.

FIGS. 7A-7G. Detection of heroin and fentanyl using aptamer-based dye-displacement assays. (A) Secondary structures of HM20 and F17 based on NUPACK predictions. (B) Scheme of the dye-displacement assay using HM20, F17, and MTC. (C) Absorbance spectra of HM20-MTC complexes challenged with various concentrations of heroin, with the pink-to-blue color gradient representing increasing heroin concentration. (D) Detection of heroin in binary mixtures of 25 μM heroin (˜33%) with 50 μM interferent (˜67%). (E) Absorbance spectra of HM20-MTC complexes in the presence of a mixture of heroin, fentanyl, and interferent as well as interferent alone. The spectra of HM20-MTC complex overlaps for all four drug mixtures and all four interferent samples. (F) Photographs of the dye-displacement assays. (G) Absorbance spectra of F17-MTC complexes in the presence of a mixture of heroin, fentanyl, and interferent as well as interferent alone. Error bars represent the standard deviation of three independent experiments.

FIGS. 8A-8I. Utilizing the dye-displacement assay and OM9 to detect oxycodone. (A) Secondary structure of OM9 based on NUPACK prediction. (B) Absorbance spectra of OM9-MTC complexes in the presence of various concentrations of oxycodone, with the pink-to-blue color gradient representing increasing concentrations. (C) Calibration curve and (D) linear range for oxycodone detection. (E) Assay specificity against 25 μM oxycodone or 50 μM interferent, with cross-reactivity shown relative to oxycodone. (F) Assay response to binary mixtures of 25 μM oxycodone and 50 μM interferent. (G) Absorbance spectra of OM9-MTC complexes challenged with oxycodone tablet extract as well as extracts of other over-the-counter drugs. (H) Quantified assay response to these extracts, and (I) photographs of the assay results. Error bars represent the standard deviation of three independent experiments. The tablets numbered 1-8 are Advil, Claritin, Benadryl, Mucinex, Tylenol, generic hydrocodone/acetaminophen, generic oxycodone/acetaminophen, respectively.

FIG. 9. Chemical structure of interferents employed in counter-SELEX.

FIGS. 10A-10D. The binding affinity of the Round P5 (A) heroin and (B) morphine pools and Round P6 (C) heroin and (D) morphine pools. All eluents were analyzed via gel-elution assay. All binding curves were fitted with a modified one-site Langmuir equation to determine K_{D}.

FIGS. 11A-11B. The binding affinity of the round S4 pool for (A) heroin and (B) morphine. All eluents were analyzed via gel-elution assay. All binding curves were fitted with a modified one-site Langmuir equation to determine K_{D}.

FIGS. 12A-12B. Binding affinity of the (A) R5 oxycodone pool and (B) R6 oxycodone pool. All eluents were analyzed via gel-elution assay. All binding curves were fitted with a modified one-site Langmuir equation to determine K_{D}.

FIGS. 13A-13B. Binding affinity of the round T6 pool for (A) oxycodone and (B) oxymorphone. All eluents were analyzed via gel-elution assay. All binding curves were fitted with a modified one-site Langmuir equation to determine K_{D}.

FIGS. 14A-14B. HTS analysis of the final pool for heroin and morphine. (A) The number of unique sequences and (B) percent of total abundance of the S4 pool belonging to each of the four categories described in the main text.

FIGS. 15A-15B. HTS analysis of the final pool for oxycodone and oxymorphone. (A) The number of unique sequences and (B) percent of total abundance of the T6 pool belonging to each of the four categories described in the main text.

FIGS. 16A-16B. Binding of MTC to HM20. (A) Absorbance spectra of solutions containing 2.5 μM MTC with 0, 1, 2, 3, 4, 5, 6, 7, 8, 9 or 10 μM HM20, where the blue-to-purple color gradient shows increasing aptamer concentrations. (B) Absorbance of MTC dimers at 550 nm and J-aggregates at 650 nm plotted against concentration of HM20.

FIGS. 17A-17D. Detection of heroin using MTC and HM20. (A) Calibration curve and (B) linear range of absorbance spectra of solutions containing 2.5 μM MTC and 6 μM HM20 challenged with 0, 0.5, 1, 2, 4, 8, 16, 32, 64, 128, 256, and 512 μM heroin. (C) Spectra of 2.5μ M MTC in the presence of 0, 0.5, 1, 2, 4, 8, 16, 32, 64, 128, 256, and 512μ M heroin, where blue-to-purple color gradient represents increasing target concentrations. (D) Photographs taken five minutes after addition of various concentrations of heroin to solutions of HM20-MTC complexes.

FIGS. 18A-18B. Specificity of an MTC-displacement assay based on aptamer HM20. (A) Signal gain and cross-reactivity of the assay to heroin and various interferents. (B) A photograph of samples taken 5 min after adding ligand to aptamer-dye complexes. [Heroin]: 25 μM. [Interferent]: 50 HM.

FIGS. 19A-19B. Detection of heroin and fentanyl using aptamer-based MTC-displacement assays. Signal gain from (A) HM20-MTC complexes and (B) F17-MTC complexes in the presence of a 33:8:59 mixture of heroin, fentanyl, and interferent (caffeine, lidocaine, lactose, or mannitol for Sample ID 1, 2, 3, or 4 respectively). The analysis was derived from FIGS. 7E and G.

FIGS. 20A-20B. Isothermal titration calorimetry (ITC) measurements of the binding affinity of (A) heroin-binding aptamer HM20 to fentanyl and (B) fentanyl-binding aptamer F17 to heroin. The top panels present the raw data showing heat generated from each titration of target into the aptamers, and bottom panels depict the integrated heat of each titration after correcting for dilution heat of titrant. Data were fitted with a single-site binding model.

FIGS. 21A-21B. Binding of MTC to OM9. (A) Absorbance spectra of solutions containing 2.5 μM MTC with 0, 1, 2, 3, 4, 5, 6, 7, 8, 9 or 10 μM OM9, where the blue-to-purple color gradient shows increasing aptamer concentration. (B) Absorbance of MTC monomers at 580 nm and J-aggregates at 650 nm plotted versus concentration of OM9.

FIG. 22. Detection of oxycodone using MTC and OM9. Photographs taken five minutes after addition of various concentrations of oxycodone (0, 0.5, 1, 2, 4, 8, 16, 32, 64, 128, 256, and 512 μM) to solutions of OM9-MTC complexes containing 2.5 μM MTC and 4 UM OM9.

FIG. 23. Effect of target on spectra of MTC-OM9 complexes. Spectra of 2.5 μM MTC in the presence of 0, 0.5, 1, 2, 4, 8, 16, 32, 64, 128, 256, and 512 μM oxycodone with no aptamer present, where the blue-to-purple color gradient represents increasing target concentrations.

### DETAILED DESCRIPTION

In accordance with the various embodiments of the present disclosure, in vitro selections were performed to isolate DNA aptamers that bind to morphine-related opioids. Using two different selection strategies, three sets of opioid-binding aptamers with distinct specificity profiles were identified. The HM series of aptamers bind to opioids containing hydrogen at C14, including morphine, codeine, heroin, hydrocodone, and hydromorphone, but do not recognize those with hydroxyl at C14, such as oxycodone and oxymorphone, or the opioid antagonists naloxone, naltrexone, and methylnaltrexone. This information, along with the fact that the aptamers tolerate substitutions at C3 and C6 and have no preference for the double bond between C7 and C8, indicates that the HM aptamers interact with the A, B, and D rings of the opioids, whereas substituents at C3 and C6 should face away from the aptamer and towards the solution. Selection with oxycodone and oxymorphone revealed two aptamers with differing opioid cross-reactivities. Aptamers such as OM9 recognize opioids with a C14 hydroxyl group, suggesting that there are important interactions between these aptamers and substituents at the B and D rings. The moderate cross-reactivity of OM9 to naloxone also demonstrates that the hydroxyl group at C14 is important for binding, but that bulky substituents at the amino group decrease affinity. The intolerance of the aptamer for bulk at the amino moiety is further evidenced by the lack of affinity for naltrexone and methylnaltrexone, which feature a methyl cyclopropyl substituent. On the other hand, aptamers like OM4 can bind oxycodone, oxymorphone, hydrocodone, and hydromorphone, but not morphine, codeine, and heroin. This indicates that the keto moiety on the C ring is a significant interaction point for this aptamer, and its tolerance to variation at C6 and C14 substituents suggests that it primarily interacts with the C and D ring systems of the opioid molecules. Notably, aptamers HM20 and OM9, among several others identified in this work, are highly specific and do not cross-react to cutting agents like caffeine and lactose; adulterants such as procaine, quinine, and acetaminophen; endogenous poppy plant constituents noscapine and papaverine; or other drugs of abuse such as cocaine, fentanyl, methamphetamine, ethylone, and various benzodiazepines.

As described further herein, a new approach to analyze the HTS data from the SELEX pools was utilized to easily identify high-affinity aptamers. Specifically, aptamer sequences were categorized based on whether they had consistently accelerated growth rates (Category 1), consistently positive but decelerating growth rates (Category 2), or growth rates that peaked before decreasing (Category 3). It was determined that aptamers in Category 1 had superior target-binding affinity to those in Category 2, and Category 3 aptamers had the lowest affinity overall. These data indicate that selecting aptamer candidates based on their growth rate throughout multiple rounds might be a promising positive predictor of aptamer quality. As part of the aptamer characterization process, as previously reported, exonuclease assays were utilized to screen the binding performance of aptamers.

Additionally, newly isolated HM20 and OM9 aptamers and the organic dye MTC were successfully utilized to develop dye-displacement assays for detecting opioids. In general, these assays are not only sensitive and specific, but also rapid—requiring only seconds for a color change—and easy to perform onsite. It has been demonstrated that these assays can detect oxycodone, heroin, and fentanyl in complex mixtures without meaningful interference or matrix effects. For instance, using HM20 and the fentanyl-binding aptamer F17, could accurately detect the presence of 33% heroin and 8% fentanyl, respectively, in the presence of ˜60% interferent without any crosstalk. It was also shown that these assays were able to identify oxycodone in prescription tablets without any false positives to commonly used over-the-counter medications including ibuprofen, loratadine, diphenhydramine, acetaminophen, and dextromethorphan/guaifenesin. These results highlight both the excellent specificity of the present aptamers and the robustness of the dye-displacement assay platform. These assays may be key for on-site drug testing relative to conventional chemical drug tests like the Marquis test because they are highly specific, and it could be anticipated that this assay could augment drug interdiction efforts and prevent harm associated with the consumption of fentanyl-adulterated substances.

### 1. DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

The term “aptamer” generally refers to either an oligonucleotide of a single defined sequence or a mixture of said oligonucleotides, wherein the mixture retains the properties of binding specifically to a target molecule. Thus, as used herein “aptamer” denotes both singular and plural sequences of oligonucleotides. The term “aptamer” generally refers to a single-stranded oligonucleotide that is capable of binding to a protein or other molecule, and thereby modulating function.

In some embodiments of the present disclosure, aptamer sequences include one or more variable nucleic acids, which are designated “X.” In some embodiments, groups of variable nucleic acids in an aptamer sequence are designated with Xₙ, or Xₙ₋ₙ, in which “n” represents an integer. For example, an aptamer sequence comprising “X₅” indicates that there are 5 consecutive variable nucleic acids in that sequence region, and each of positions 1-5 have one or more possible nucleic acids at that position (e.g., variable). Additionally, an aptamer sequence comprising “X₃₋₅,” for example, indicates that each of nucleic acids 1-3 are present in that nucleic acid molecule, but the nucleic acids at position 4 and 5 are optional.

The term “single-stranded” oligonucleotides generally refers to those oligonucleotides that contain a single covalently linked series of nucleotide residues.

The terms “oligomers” or “oligonucleotides” include RNA or DNA sequences of more than one nucleotide in either single chain or duplex form and specifically includes short sequences such as dimers and trimers, in either single chain or duplex form, which can be intermediates in the production of the specifically binding oligonucleotides. “Modified” forms used in candidate pools contain at least one non-native residue. “Oligonucleotide” or “oligomer” is generic to polydeoxyribonucleotides (containing 2′-deoxy-D-ribose or modified forms thereof), such as DNA, to polyribonucleotides (containing D-ribose or modified forms thereof), such as RNA, and to any other type of polynucleotide which is an N-glycoside or C-glycoside of a purine or pyrimidine base, or modified purine or pyrimidine base or abasic nucleotides. “Oligonucleotide” or “oligomer” can also be used to describe artificially synthesized polymers that are similar to RNA and DNA, including, but not limited to, oligos of peptide nucleic acids (PNA).

The terms “binding activity” and “binding affinity” generally refer to the tendency of a ligand molecule to bind or not to bind to a target. The energetics of these interactions are significant in “binding activity” and “binding affinity” because they can include definitions of the concentrations of interacting partners, the rates at which these partners are capable of associating, and the relative concentrations of bound and free molecules in a solution.

The terms “specific binding” and “specifically binding” and derivatives thereof refer to molecules that exhibit high substrate specificity for a given target and very low or no substrate specificity for any other target within an operating concentration range.

“Complementary” refers to the characteristic of two or more structural elements (e.g., peptide, polypeptide, nucleic acid, small molecule, etc.) of being able to hybridize, dimerize, or otherwise form a complex with each other. For example, a “complementary peptide and polypeptide” are capable of coming together to form a complex. Complementary elements may require assistance to form a complex (e.g., from interaction elements), for example, to place the elements in the proper conformation for complementarity, to co-localize complementary elements, to lower interaction energy for complementation, etc.

As used herein, the terms “nucleotide sequence identity” or “nucleic acid sequence identity” refers to the presence of identical nucleotides at corresponding positions of two polynucleotides. Polynucleotides have “identical” sequences if the sequence of nucleotides in the two polynucleotides is the same when aligned for maximum correspondence (e.g., in a comparison window). Sequence comparison between two or more polynucleotides is generally performed by comparing portions of the two sequences over a comparison window to identify and compare local regions of sequence similarity. The comparison window is generally from about 20 to 200 contiguous nucleotides. The “percentage of sequence identity” for polynucleotides, such as about 5, 10, 15, 20, 25, 30, 35, 40, 45, 50, 55, 60, 65, 70, 75, 80, 85, 90, 95, 98, 99 or 100 percent sequence identity, can be determined by comparing two optimally aligned sequences over a comparison window, wherein the portion of the polynucleotide sequence in the comparison window can include additions or deletions (i.e., gaps) as compared to the reference sequence for optimal alignment of the two sequences. In some embodiments, the percentage is calculated by: (a) determining the number of positions at which the identical nucleic acid base occurs in both sequences; (b) dividing the number of matched positions by the total number of positions in the window of comparison; and (c) multiplying the result by 100. Optimal alignment of sequences for comparison can also be conducted by computerized implementations of known algorithms, or by visual inspection. Readily available sequence comparison and multiple sequence alignment algorithms are, respectively, the Basic Local Alignment Search Tool (BLAST) and ClustalW/ClustalW2/Clustal Omega programs available on the Internet (e.g., the website of the EMBL-EBI). Other suitable programs include, but are not limited to, GAP, BestFit, Plot Similarity, and FASTA, which are part of the Accelrys GCG Package available from Accelrys, Inc. of San Diego, Calif., United States of America. See also Smith & Waterman, 1981; Needleman & Wunsch, 1970; Pearson & Lipman, 1988; Ausubel et al., 1988; and Sambrook & Russell, 2001.

### 2. APTAMERS

In accordance with the various embodiments of the present disclosure, described herein are methods and compositions pertaining to colorimetric dye-displacement assays. In particular, the present disclosure provides aptamer-based colorimetric assays enabled for visualization, by the naked-eye, for the identification of specific opioids (natural, semi-synthetic, synthetic and the derivatives and analogs thereof) in a manner that is rapid, specific, and sensitive.

In some embodiments of the present disclosure, the compositions described herein include a single-stranded nucleic acid molecule capable of specifically binding heroin or morphine, or a derivative or analog thereof, comprising a nucleic acid sequence that is at least 50% identical to the following: GGATTCG(X)₁₆₋₁₈CTCGT; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; X₁₅ is A, T, C, or G; X₁₆ is A, T, C, or G; X₁₇ is A, T, C, or G; and X₁₈ is A, T, C, or G (SEQ ID NO: 1). In some embodiments of the present disclosure, the nucleic at least 60% identical to the following: GGATTCG(X)₁₆₋₁₈CTCGT; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; X₁₅ is A, T, C, or G; X₁₆ is A, T, C, or G; X₁₇ is A, T, C, or G; and X₁₈ is A, T, C, or G (SEQ ID NO: 1). In some embodiments of the present disclosure, the nucleic at least 70% identical to the following: GGATTCG(X)₁₆₋₁₈CTCGT; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; X₁₅ is A, T, C, or G; X₁₆ is A, T, C, or G; X₁₇ is A, T, C, or G; and X₁₈ is A, T, C, or G (SEQ ID NO: 1). In some embodiments of the present disclosure, the nucleic at least 80% identical to the following: GGATTCG(X)₁₆₋₁₈CTCGT; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; X₁₅ is A, T, C, or G; X₁₆ is A, T, C, or G; X₁₇ is A, T, C, or G; and X₁₈ is A, T, C, or G (SEQ ID NO: 1). In some embodiments of the present disclosure, the nucleic at least 90% identical to the following: GGATTC(X)₁₆₋₁₈CTCGT; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; X₁₅ is A, T, C, or G; X₁₆ is A, T, C, or G; X₁₇ is A, T, C, or G; and X₁₈ is A, T, C, or G (SEQ ID NO: 1). In accordance with these embodiments, (X)₁₆₋₁₈ indicates that X₁₋₁₆ are present in the nucleic acid molecule, but X₁₇ and X₁₈ are optional.

In some embodiments, the nucleic sequence is from about 50% to about 90% identical to the following: GGATTCG(X)₁₆₋₁₈CTCGT; wherein X₁ is G or C; X₂ is A or G; X₃ is T or C; X₄ is C; X₅ is G or C; X₆ is T or C; X₇ is G or A; X₈ is G or A; X₉ is A or G; X₁₀ is A or T; X₁₁ is C or G; X₁₂ is A or G; X₁₃ is G; X₁₄ is T or G; X₁₅ is G or A; X₁₆ is C or G; X₁₇ is G; and X₁₈ is G (SEQ ID NO: 2). In accordance with these embodiments, (X)₁₆₋₁₈ indicates that X₁₋₁₆ are present in the nucleic acid molecule, but X₁₇ and X₁₈ are optional.

In some embodiments, the nucleic acid molecule comprises a nucleic acid sequence that is at least 50% identical to SEQ ID NOs: 9 or 10. In some embodiments, the nucleic acid molecule comprises a nucleic acid sequence that is at least 60% identical to SEQ ID NOs: 9 or 10. In some embodiments, the nucleic acid molecule comprises a nucleic acid sequence that is at least 70% identical to SEQ ID NOs: 9 or 10. In some embodiments, the nucleic acid molecule comprises a nucleic acid sequence that is at least 80% identical to SEQ ID NOs: 9 or 10. In some embodiments, the nucleic acid molecule comprises a nucleic acid sequence that is at least 90% identical to SEQ ID NOs: 9 or 10.

In accordance with these embodiments, the nucleic acid molecule comprises a K_{D} that is less than about 900 nM. In some embodiments, the nucleic acid molecule comprises a K_{D} that is less than about 850 nM. In some embodiments, the nucleic acid molecule comprises a K_{D} that is less than about 750 nM. In some embodiments, the nucleic acid molecule comprises a K_{D} that is less than about 650 nM. In some embodiments, the nucleic acid molecule comprises a K_{D} that is less than about 550 nM. In some embodiments, the nucleic acid molecule comprises a K_{D} that is less than about 450 nM. In some embodiments, the nucleic acid molecule comprises a K_{D} that is less than about 350 nM. In some embodiments, the nucleic acid molecule comprises a K_{D} that is less than about 250 nM. In some embodiments, the nucleic acid molecule comprises a K_{D} that is less than about 150 nM. In some embodiments, the nucleic acid molecule comprises a K_{D} that is less than about 100 nM. In some embodiments, the nucleic acid molecule comprises a K_{D} that is less than about 50 nM. In some embodiments, the nucleic acid molecule comprises a K_{D} that is less than about 25 nM.

Embodiments of the present disclosure also include a single-stranded nucleic acid molecule capable of specifically binding heroin or morphine, or a derivative or analog thereof, comprising a nucleic acid sequence that is at least 50% identical to the following: GCGTA(X)₇TAG(X)₅CGTCGTTCAA; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; and X₁₂ is A, T, C, or G (SEQ ID NO: 3). In some embodiments, the nucleic acid sequence is at least 60% identical to the following: GCGTA(X)₇TAG(X)₅CGTCGTTCAA; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; and X₁₂ is A, T, C, or G (SEQ ID NO: 3). In some embodiments, the nucleic acid is at least 70% identical to sequence the following: GCGTA(X)₇TAG(X)₅CGTCGTTCAA; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; and X₁₂ is A, T, C, or G (SEQ ID NO: 3). In some embodiments, the nucleic acid sequence is at least 80% identical to the following: GCGTA(X)₇TAG(X)₅CGTCGTTCAA; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; and X₁₂ is A, T, C, or G (SEQ ID NO: 3). In some embodiments, the nucleic acid is sequence at least 90% identical to the following: GCGTA(X)₇TAG(X)₅CGTCGTTCAA; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; and X₁₂ is A, T, C, or G (SEQ ID NO: 3).

In some embodiments, the nucleic acid sequence is from about 50% to about 90% identical to the following: GCGTA(X)₇TAG(X)₅CGTCGTTCAA; wherein X₁ is G or C; X₂ is G, C, or T; X₃ is T or A; X₄ is T, C, or G; X₅ is T, C, or G; X₆ is C, G, or A; X₇ is C or G; X₈ is C or T; X₉ is T or C; X₁₀ is G; X₁₁ is T or C; and X₁₂ is G or A (SEQ ID NO: 4).

In some embodiments, the nucleic acid molecule comprises a nucleic acid sequence that is at least 50% identical to any one of SEQ ID NOs: 11-17. In some embodiments, the nucleic acid molecule comprises a nucleic acid sequence that is at least 60% identical to any one of SEQ ID NOs: 11-17. In some embodiments, the nucleic acid molecule comprises a nucleic acid sequence that is at least 70% identical to any one of SEQ ID NOs: 11-17. In some embodiments, the nucleic acid molecule comprises a nucleic acid sequence that is at least 80% identical to any one of SEQ ID NOs: 11-17. In some embodiments, the nucleic acid molecule comprises a nucleic acid sequence that is at least 90% identical to any one of SEQ ID NOs: 11-17.

In accordance with these embodiments, the nucleic acid molecule comprises a K_{D} that is less than about 6000 nM. In some embodiments, the nucleic acid molecule comprises a K_{D} that is less than about 5000 nM. In some embodiments, the nucleic acid molecule comprises a K_{D} that is less than about 4000 nM. In some embodiments, the nucleic acid molecule comprises a K_{D} that is less than about 3000 nM. In some embodiments, the nucleic acid molecule comprises a K_{D} that is less than about 2000 nM. In some embodiments, the nucleic acid molecule comprises a K_{D} that is less than about 1000 nM. In some embodiments, the nucleic acid molecule comprises a K_{D} that is less than about 900 nM. In some embodiments, the nucleic acid molecule comprises a K_{D} that is less than about 800 nM. In some embodiments, the nucleic acid molecule comprises a K_{D} that is less than about 700 nM. In some embodiments, the nucleic acid molecule comprises a K_{D} that is less than about 600 nM. In some embodiments, the nucleic acid molecule comprises a K_{D} that is less than about 500 nM. In some embodiments, the nucleic acid molecule comprises a K_{D} that is less than about 400 nM. In some embodiments, the nucleic acid molecule comprises a K_{D} that is less than about 300 nM. In some embodiments, the nucleic acid molecule comprises a K_{D} that is less than about 200 nM. In some embodiments, the nucleic acid molecule comprises a K_{D} that is less than about 100 nM. In some embodiments, the nucleic acid molecule comprises a K_{D} that is less than about 50 nM. In some embodiments, the nucleic acid molecule comprises a K_{D} that is less than about 25 nM.

Embodiments of the present disclosure also include a single-stranded nucleic acid molecule capable of specifically binding heroin or morphine, or a derivative or analog thereof, comprising a nucleic acid sequence that is at least 50% identical to the following: TAGC(X)₃₋₄GCGTTGTTCGA(X)₆₋₈AGTA; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; and X₁₂ is A, T, C, or G (SEQ ID NO: 5). In some embodiments, the nucleic acid is at least 60% identical to the following: TAGC(X)₃₋₄GCGTTGTTCGA(X)₆₋₈AGTA; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; and X₁₂ is A, T, C, or G (SEQ ID NO: 5). In some embodiments, the nucleic acid is at least 70% identical to the following: TAGC(X)₃₋₄GCGTTGTTCGA(X)₆₋₈AGTA; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; and X 12 is A, T, C, or G (SEQ ID NO: 5). In some embodiments, the nucleic acid is at least 80% identical to the following: TAGC(X)₃₋₄GCGTTGTTCGA(X)₆₋₈AGTA; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; and X 12 is A, T, C, or G (SEQ ID NO: 5). In some embodiments, the nucleic acid is at least 90% identical to the following: TAGC(X)₃₋₄GCGTTGTTCGA(X)₆₋₈AGTA; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; and X₁₂ is A, T, C, or G (SEQ ID NO: 5). In accordance with these embodiments, (X) 34 indicates that X₁₋₃ are present in the nucleic acid molecule, but X₄ is optional. Additionally, (X)₆₋₈ indicates that X₁₆ are present in the nucleic acid molecule, but X₇ and X₈ are optional.

In some embodiments, the nucleic acid sequence is from about 50% to about 90% identical to the following: TAGC(X)₃₋₄GCGTTGTTCGA(X)₆₋₈AGTA; wherein X₁ is C, G, or T; X₂ is C or G; X₃ is A, T, C, or G; X₄ is G, T, or A; X₅ is G, A, or C; X₆ is T, C, or G; X₇ is A, T, C, or G; X₈ is C, A, or T; X₉ is A, G, or T; X₁₀ is G, T, or C; X₁₁ is C or G; and X₁₂ is G or A (SEQ ID NO: 6). In accordance with these embodiments, (X)₃₋₄ indicates that X₁₋₃ are present in the nucleic acid molecule, but X₄ is optional. Additionally, (X)₆₋₈ indicates that X₁₆ are present in the nucleic acid molecule, but X₇ and X₈ are optional.

In some embodiments, the nucleic acid molecule comprises a nucleic acid sequence that is at least 50% identical to any one of SEQ ID NOs: 18-26. In some embodiments, the nucleic acid molecule comprises a nucleic acid sequence that is at least 60% identical to any one of SEQ ID NOs: 18-26. In some embodiments, the nucleic acid molecule comprises a nucleic acid sequence that is at least 70% identical to any one of SEQ ID NOs: 18-26. In some embodiments, the nucleic acid molecule comprises a nucleic acid sequence that is at least 80% identical to any one of SEQ ID NOs: 18-26. In some embodiments, the nucleic acid molecule comprises a nucleic acid sequence that is at least 90% identical to any one of SEQ ID NOs: 18-26.

In accordance with these embodiments, the nucleic acid molecule comprises a K_{D} that is less than about 3500 nM. In some embodiments, the nucleic acid molecule comprises a K_{D} that is less than about 3000 nM. In some embodiments, the nucleic acid molecule comprises a K_{D} that is less than about 2500 nM. In some embodiments, the nucleic acid molecule comprises a K_{D} that is less than about 2000 nM. In some embodiments, the nucleic acid molecule comprises a K_{D} that is less than about 1500 nM. In some embodiments, the nucleic acid molecule comprises a K_{D} that is less than about 1000 nM. In some embodiments, the nucleic acid molecule comprises a K_{D} that is less than about 900 nM. In some embodiments, the nucleic acid molecule comprises a K_{D} that is less than about 800 nM. In some embodiments, the nucleic acid molecule comprises a K_{D} that is less than about 700 nM. In some embodiments, the nucleic acid molecule comprises a K_{D} that is less than about 600 nM. In some embodiments, the nucleic acid molecule comprises a K_{D} that is less than about 500 nM. In some embodiments, the nucleic acid molecule comprises a K_{D} that is less than about 400 nM. In some embodiments, the nucleic acid molecule comprises a K_{D} that is less than about 300 nM. In some embodiments, the nucleic acid molecule comprises a K_{D} that is less than about 200 nM. In some embodiments, the nucleic acid molecule comprises a K_{D} that is less than about 100 nM. In some embodiments, the nucleic acid molecule comprises a K_{D} that is less than about 50 nM. In some embodiments, the nucleic acid molecule comprises a K_{D} that is less than about 25 nM.

Embodiments of the present disclosure also include a single-stranded nucleic acid molecule capable of specifically binding heroin or morphine, or a derivative or analog thereof, comprising a nucleic acid sequence that is at least 50% identical to the following: AGGGCACGTCT(X)₆₋₈AGGGTTTCGCG; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; and X₈ is A, T, C, or G (SEQ ID NO: 7). In some embodiments, the nucleic acid is at least 60% identical to the following: AGGGCACGTCT(X)₆₋₈AGGGTTTCGCG; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; and X₈ is A, T, C, or G (SEQ ID NO: 7). In some embodiments, the nucleic acid is at least 70% identical to the following: AGGGCACGTCT(X)₆₋₈AGGGTTTCGCG; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; and X₈ is A, T, C, or G (SEQ ID NO: 7). In some embodiments, the nucleic acid is at least 80% identical to the following: AGGGCACGTCT(X)₆₋₈AGGGTTTCGCG; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; and X₈ is A, T, C, or G (SEQ ID NO: 7). In some embodiments, the nucleic acid is at least 90% identical to the following: AGGGCACGTCT(X)₆₋₈AGGGTTTCGCG; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; and X₈ is A, T, C, or G (SEQ ID NO: 7). In accordance with these embodiments, (X)₆₋₈ indicates that X₁₆ are present in the nucleic acid molecule, but X₇ and X₈ are optional.

In some embodiments, the nucleic sequence is from about 50% to about 90% identical to the following: AGGGCACGTCT(X)₆₋₈AGGGTTTCGCG; X₁ is C or G; X₂ is G or T; X₃ is G; X₄ is C or T; X₅ is A or G; X₆ is T or C; X₇ is C; and X₈ is G (SEQ ID NO: 8). In accordance with these embodiments, (X)₆₋₈ indicates that X₁₆ are present in the nucleic acid molecule, but X₇ and X₈ are optional.

In some embodiments, the nucleic acid molecule comprises a nucleic acid sequence that is at least 50% identical to SEQ ID NOs: 27 or 28. In some embodiments, the nucleic acid molecule comprises a nucleic acid sequence that is at least 60% identical to SEQ ID NOs: 27 or 28. In some embodiments, the nucleic acid molecule comprises a nucleic acid sequence that is at least 70% identical to SEQ ID NOs: 27 or 28. In some embodiments, the nucleic acid molecule comprises a nucleic acid sequence that is at least 80% identical to SEQ ID NOs: 27 or 28. In some embodiments, the nucleic acid molecule comprises a nucleic acid sequence that is at least 90% identical to SEQ ID NOs: 27 or 28.

In accordance with these embodiments, the nucleic acid molecule comprises a K_{D} that is less than about 5000 nM. In some embodiments, the nucleic acid molecule comprises a K_{D} that is less than about 4000 nM. In some embodiments, the nucleic acid molecule comprises a K_{D} that is less than about 3000 nM. In some embodiments, the nucleic acid molecule comprises a K_{D} that is less than about 2000 nM. In some embodiments, the nucleic acid molecule comprises a K_{D} that is less than about 1000 nM. In some embodiments, the nucleic acid molecule comprises a K_{D} that is less than about 900 nM. In some embodiments, the nucleic acid molecule comprises a K_{D} that is less than about 800 nM. In some embodiments, the nucleic acid molecule comprises a K_{D} that is less than about 700 nM. In some embodiments, the nucleic acid molecule comprises a K_{D} that is less than about 600 nM. In some embodiments, the nucleic acid molecule comprises a K_{D} that is less than about 500 nM. In some embodiments, the nucleic acid molecule comprises a K_{D} that is less than about 400 nM. In some embodiments, the nucleic acid molecule comprises a K_{D} that is less than about 300 nM. In some embodiments, the nucleic acid molecule comprises a K_{D} that is less than about 200 nM. In some embodiments, the nucleic acid molecule comprises a K_{D} that is less than about 100 nM. In some embodiments, the nucleic acid molecule comprises a K_{D} that is less than about 50 nM. In some embodiments, the nucleic acid molecule comprises a K_{D} that is less than about 25 nM.

In some embodiments, a single-stranded nucleic acid molecule capable of specifically binding heroin or morphine, or a derivative or analog thereof, comprising a nucleic acid sequence that is at least 50% identical to any one of SEQ ID NOs: 29-33. In some embodiments, a single-stranded nucleic acid molecule capable of specifically binding heroin or morphine, or a derivative or analog thereof, comprising a nucleic acid sequence that is at least 60% identical to any one of SEQ ID NOs: 29-33. In some embodiments, a single-stranded nucleic acid molecule capable of specifically binding heroin or morphine, or a derivative or analog thereof, comprising a nucleic acid sequence that is at least 70% identical to any one of SEQ ID NOs: 29-33. In some embodiments, a single-stranded nucleic acid molecule capable of specifically binding heroin or morphine, or a derivative or analog thereof, comprising a nucleic acid sequence that is at least 80% identical to any one of SEQ ID NOs: 29-33. In some embodiments, a single-stranded nucleic acid molecule capable of specifically binding heroin or morphine, or a derivative or analog thereof, comprising a nucleic acid sequence that is at least 90% identical to any one of SEQ ID NOs: 29-33.

In accordance with embodiments, the single-stranded nucleic acid molecule capable of specifically binding oxycodone or oxymorphone, or a derivative or analog thereof, comprising a nucleic acid sequence that is at least 50% identical to the following: (X)₁₂₋₁₃TCTCAGCGAGTTCG(X)₃₋₄; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; X₁₅ is A, T, C, or G; X₁₆ is A, T, C, or G; and X₁₇ is A, T, C, or G (SEQ ID NO: 34). In some embodiments, the nucleic acid is at least 60% identical to the following: (X)₁₂₋₁₃TCTCAGCGAGTTCG(X)₃₋₄; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; X₁₅ is A, T, C, or G; X₁₆ is A, T, C, or G; and X₁₇ is A, T, C, or G (SEQ ID NO: 34). In some embodiments, the nucleic acid is at least 70% identical to the following: (X)₁₂₋₁₃TCTCAGCGAGTTCG(X)₃₋₄; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; X₁₅ is A, T, C, or G; X₁₆ is A, T, C, or G; and X₁₇ is A, T, C, or G (SEQ ID NO: 34). In some embodiments, the nucleic acid is at least 80% identical to the following: (X)₁₂₋₁₃TCTCAGCGAGTTCG(X)₃₋₄; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; X₁₅ is A, T, C, or G; X₁₆ is A, T, C, or G; and X 17 is A, T, C, or G (SEQ ID NO: 34). In some embodiments, the nucleic acid is at least 90% identical to the following: (X)₁₂₋₁₃TCTCAGCGAGTTCG(X)₃₋₄; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; X₁₅ is A, T, C, or G; X₁₆ is A, T, C, or G; and X₁₇ is A, T, C, or G (SEQ ID NO: 34). In accordance with these embodiments, (X)₁₂₋₁₃ indicates that X₁₋₁₂ are present in the nucleic acid molecule, but X₁₃ is optional. Additionally, (X)₃₋₄ indicates that X₁₋₃ are present in the nucleic acid molecule, but X₄ is optional.

In some embodiments, the nucleic sequence is from about 50% to about 90% identical to the following: (X)₁₂₋₁₃TCTCAGCGAGTTCG(X)₃₋₄; wherein X₁ is A, T, or G; X₂ is A, C, or G; X₃ is G, C, or A; X₄ is T or G; X₅ is C or T; X₆ is A; X₇ is G; X₈ is G, C, or T; X₉ is C or T; X₁₀ is C; X₁₁ is T or A; X₁₂ is G or T; X₁₃ is C or G; X₁₄ is A, C, or T; X₁₅ is C, G, or T; X₁₆ is A or G; and X₁₇ is T or A (SEQ ID NO: 35). In accordance with these embodiments, (X)₁₂₋₁₃ indicates that X₁₋₁₂ are present in the nucleic acid molecule, but X₁₃ is optional. Additionally, (X)₃₋₄ indicates that X₁₋₃ are present in the nucleic acid molecule, but X₄ is optional.

In some embodiments, the nucleic acid molecule comprises a nucleic acid sequence that is at least 50% identical to any one of SEQ ID NOs: 46-51. In some embodiments, the nucleic acid molecule comprises a nucleic acid sequence that is at least 60% identical to any one of SEQ ID NOs: 46-51. In some embodiments, the nucleic acid molecule comprises a nucleic acid sequence that is at least 70% identical to any one of SEQ ID NOs: 46-51. In some embodiments, the nucleic acid molecule comprises a nucleic acid sequence that is at least 80% identical to any one of SEQ ID NOs: 46-51. In some embodiments, the nucleic acid molecule comprises a nucleic acid sequence that is at least 90% identical to any one of SEQ ID NOs: 46-51.

In accordance with these embodiments, the nucleic acid molecule comprises a K_{D} that is less than about 2500 nM. In some embodiments, the nucleic acid molecule comprises a K_{D} that is less than about 2000 nM. In some embodiments, the nucleic acid molecule comprises a K_{D} that is less than about 1500 nM. In some embodiments, the nucleic acid molecule comprises a K_{D} that is less than about 1000 nM. In some embodiments, the nucleic acid molecule comprises a K_{D} that is less than about 900 nM. In some embodiments, the nucleic acid molecule comprises a K_{D} that is less than about 800 nM. In some embodiments, the nucleic acid molecule comprises a K_{D} that is less than about 700 nM. In some embodiments, the nucleic acid molecule comprises a K_{D} that is less than about 600 nM. In some embodiments, the nucleic acid molecule comprises a K_{D} that is less than about 500 nM. In some embodiments, the nucleic acid molecule comprises a K_{D} that is less than about 400 nM. In some embodiments, the nucleic acid molecule comprises a K_{D} that is less than about 300 nM. In some embodiments, the nucleic acid molecule comprises a K_{D} that is less than about 200 nM. In some embodiments, the nucleic acid molecule comprises a K_{D} that is less than about 100 nM. In some embodiments, the nucleic acid molecule comprises a K_{D} that is less than about 50 nM. In some embodiments, the nucleic acid molecule comprises a K_{D} that is less than about 25 nM.

Embodiments of the present disclosure also include a single-stranded nucleic acid molecule capable of specifically binding oxycodone or oxymorphone, or a derivative or analog thereof, comprising a nucleic acid sequence that is at least 50% identical to the following: GGCT(X)₁₁GTAGGGGT(X)₁CACGCT; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; and X₁₂ is A, T, C, or G (SEQ ID NO: 36). In some embodiments, the nucleic acid is at least 60% identical to the following: GGCT(X)₁₁GTAGGGGT(X)₁CACGCT; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; and X₁₂ is A, T, C, or G (SEQ ID NO: 36). In some embodiments, the nucleic acid is at least 70% identical to the following: GGCT(X)₁₁GTAGGGGT(X)₁CACGCT; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; and X₁₂ is A, T, C, or G (SEQ ID NO: 36). In some embodiments, the nucleic acid is at least 80% identical to the following: GGCT(X)₁₁GTAGGGGT(X)₁CACGCT; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; and X₁₂ is A, T, C, or G (SEQ ID NO: 36). In some embodiments, the nucleic acid is at least 90% identical to the following: GGCT(X)₁₁GTAGGGGT(X)₁CACGCT; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; and X 12 is A, T, C, or G (SEQ ID NO: 36).

In some embodiments, the nucleic sequence is from about 50% to about 90% identical to the following: GGCT(X)₁₁GTAGGGGT(X)₁CACGCT; wherein X₁ is T; X₂ is A or G; X₃ is G or C; X₄ is C or A; X₅ is A or T; X₆ is A or T; X₇ is A or G; X₈ is G or A; X₉ is C or G; X₁₀ is T or C; X₁₁ is A; and X 12 is C or A (SEQ ID NO: 37).

In some embodiments, the nucleic acid molecule comprises a nucleic acid sequence that is at least 50% identical to SEQ ID NOs: 52 or 53. In some embodiments, the nucleic acid molecule comprises a nucleic acid sequence that is at least 60% identical to SEQ ID NOs: 52 or 53. In some embodiments, the nucleic acid molecule comprises a nucleic acid sequence that is at least 70% identical to SEQ ID NOs: 52 or 53. In some embodiments, the nucleic acid molecule comprises a nucleic acid sequence that is at least 80% identical to SEQ ID NOs: 52 or 53. In some embodiments, the nucleic acid molecule comprises a nucleic acid sequence that is at least 90% identical to SEQ ID NOs: 52 or 53.

In accordance with these embodiments, the nucleic acid molecule comprises a K_{D} that is less than about 1000 nM. In some embodiments, the nucleic acid molecule comprises a K_{D} that is less than about 900 nM. In some embodiments, the nucleic acid molecule comprises a K_{D} that is less than about 800 nM. In some embodiments, the nucleic acid molecule comprises a K_{D} that is less than about 700 nM. In some embodiments, the nucleic acid molecule comprises a K_{D} that is less than about 600 nM. In some embodiments, the nucleic acid molecule comprises a K_{D} that is less than about 500 nM. In some embodiments, the nucleic acid molecule comprises a K_{D} that is less than about 400 nM. In some embodiments, the nucleic acid molecule comprises a K_{D} that is less than about 300 nM. In some embodiments, the nucleic acid molecule comprises a K_{D} that is less than about 200 nM. In some embodiments, the nucleic acid molecule comprises a K_{D} that is less than about 100 nM. In some embodiments, the nucleic acid molecule comprises a K_{D} that is less than about 50 nM. In some embodiments, the nucleic acid molecule comprises a K_{D} that is less than about 25 nM.

Embodiments of the present disclosure also include a single-stranded nucleic acid molecule capable of specifically binding oxycodone or oxymorphone, or a derivative or analog thereof, comprising a nucleic acid sequence that is at least 50% identical to the following: (X)₂₋₆GGGAGT(X)₂₋₃GTTTGTGTGGGG(X)₂₋₆; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; and X₁₅ is A, T, C, or G (SEQ ID NO: 38). In some embodiments, the nucleic acid is at least 60% identical to the following: (X)₂₋₆GGGAGT(X)₂₋₃GTTTGTGTGGGG(X)₂₋₆; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; and X₁₅ is A, T, C, or G (SEQ ID NO: 38). In some embodiments, the nucleic acid is at least 70% identical to the following: (X)₂₋₆GGGAGT(X)₂₋₃GTTTGTGTGGGG(X)₂₋₆; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; and X₁₅ is A, T, C, or G (SEQ ID NO: 38). In some embodiments, the nucleic acid is at least 80% identical to the following: (X)₂₋₆GGGAGT(X)₂₋₃GTTTGTGTGGGG(X)₂₋₆; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; and X₁₅ is A, T, C, or G (SEQ ID NO: 38). In some embodiments, the nucleic acid is at least 90% identical to the following: (X)₂₋₆GGGAGT(X)₂₋₃GTTTGTGTGGGG(X)₂₋₆; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; and X₁₅ is A, T, C, or G (SEQ ID NO: 38). In accordance with these embodiments, (X)₂₋₃ indicates that X₁₋₂ are present in the nucleic acid molecule, but X₃ is optional. Additionally, (X)₂₋₆ indicates that X₁₋₂ are present in the nucleic acid molecule, but X₃, X₄, X₅, and X₆ are optional.

In some embodiments, the nucleic sequence is from about 50% to about 90% identical to the following: (X)₂₋₆GGGAGT(X)₂₋₃GTTTGTGTGGGG(X)₂₋₆; wherein X₁ is G; X₂ is A or G; X₃ is G; X₄ is T; X₅ is G; X₆ is C; X₇ is G, A, or T; X₈ is T or A; X₉ is T; X₁₀ is T, A, or G; X₁₁ is C or G; X₁₂ is C or T; X₁₃ is C; X₁₄ is G or C; and X₁₅ is A (SEQ ID NO: 39). In accordance with these embodiments, (X)₂₋₃ indicates that X₁₋₂ are present in the nucleic acid molecule, but X₃ is optional. Additionally, (X)₂₋₆ indicates that X₁₋₂ are present in the nucleic acid molecule, but X₃, X₄, X₅, and X₆ are optional.

In some embodiments, the nucleic acid molecule comprises a nucleic acid sequence that is at least 50% identical to any one of SEQ ID NOs: 54-56. In some embodiments, the nucleic acid molecule comprises a nucleic acid sequence that is at least 60% identical to any one of SEQ ID NOs: 54-56. In some embodiments, the nucleic acid molecule comprises a nucleic acid sequence that is at least 70% identical to any one of SEQ ID NOs: 54-56. In some embodiments, the nucleic acid molecule comprises a nucleic acid sequence that is at least 80% identical to any one of SEQ ID NOs: 54-56. In some embodiments, the nucleic acid molecule comprises a nucleic acid sequence that is at least 90% identical to any one of SEQ ID NOs: 54-56.

In accordance with these embodiments, the nucleic acid molecule comprises a K_{D} that is less than about 2000 nM. In some embodiments, the nucleic acid molecule comprises a K_{D} that is less than about 1500 nM. In some embodiments, the nucleic acid molecule comprises a K_{D} that is less than about 1000 nM. In some embodiments, the nucleic acid molecule comprises a K_{D} that is less than about 900 nM. In some embodiments, the nucleic acid molecule comprises a K_{D} that is less than about 800 nM. In some embodiments, the nucleic acid molecule comprises a K_{D} that is less than about 700 nM. In some embodiments, the nucleic acid molecule comprises a K_{D} that is less than about 600 nM. In some embodiments, the nucleic acid molecule comprises a K_{D} that is less than about 500 nM. In some embodiments, the nucleic acid molecule comprises a K_{D} that is less than about 400 nM. In some embodiments, the nucleic acid molecule comprises a K_{D} that is less than about 300 nM. In some embodiments, the nucleic acid molecule comprises a K_{D} that is less than about 200 nM. In some embodiments, the nucleic acid molecule comprises a K_{D} that is less than about 100 nM. In some embodiments, the nucleic acid molecule comprises a K_{D} that is less than about 50 nM. In some embodiments, the nucleic acid molecule comprises a K_{D} that is less than about 25 nM.

Embodiments of the present disclosure also include a single-stranded nucleic acid molecule capable of specifically binding oxycodone or oxymorphone, or a derivative or analog thereof, comprising a nucleic acid sequence that is at least 50% identical to the following: (X)₁₋₂CGTAGGGG(X)₁ACACGCT(X)₄₋₆GGGCTG(X)₁₋₂; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; and X₁₁ is A, T, C, or G (SEQ ID NO: 40). In some embodiments, the nucleic acid is at least 60% identical to the following: (X)₁₋₂CGTAGGGG(X)₁ACACGCT(X)₄₋₆GGGCTG(X)₁₋₂; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; and X₁₁ is A, T, C, or G (SEQ ID NO: 40). In some embodiments, the nucleic acid is at least 70% identical to the following: (X)₁₋₂CGTAGGGG(X)₁ACACGCT(X)₄₋₆GGGCTG(X)₁₋₂; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; and X₁₁ is A, T, C, or G (SEQ ID NO: 40). In some embodiments, the nucleic acid is at least 80% identical to the following: (X)₁₋₂CGTAGGGG(X)₁ACACGCT(X)₄₋₆GGGCTG(X)₁₋₂; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; and X₁₁ is A, T, C, or G (SEQ ID NO: 40). In some embodiments, the nucleic acid is at least 90% identical to the following: (X)₁₋₂CGTAGGGG(X)₁ACACGCT(X)₄₋₆GGGCTG(X)₁₋₂; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; and X₁₁ is A, T, C, or G (SEQ ID NO: 40). In accordance with these embodiments, (X)₁₋₂ indicates that X₁ is present in the nucleic acid molecule, but X₂ is optional. Additionally, (X)₄₋₆ indicates that X₄ is present in the nucleic acid molecule, but X₅ and X₆ are optional.

In some embodiments, the nucleic sequence is from about 50% to about 90% identical to the following: (X)₁₋₂CGTAGGGG(X)₁ACACGCT(X)₄₋₆GGGCTG(X)₁₋₂; wherein X₁ is C or A; X₂ is T or G; X₃ is T, C, or A; X₄ is C or T; X₅ is T, A, or G; X₆ is A or C; X₇ is T, C, or A; X₈ is A or G; X₉ is G; X₁₀ is G or A; and X₁₁ is T or G (SEQ ID NO: 41). In accordance with these embodiments, (X)₁₋₂ indicates that X₁ is present in the nucleic acid molecule, but X₂ is optional. Additionally, (X)₄₋₆ indicates that X₄ is present in the nucleic acid molecule, but X₅ and X₆ are optional.

In some embodiments, the nucleic acid molecule comprises a nucleic acid sequence that is at least 50% identical to any one of SEQ ID NOs: 57-60. In some embodiments, the nucleic acid molecule comprises a nucleic acid sequence that is at least 60% identical to any one of SEQ ID NOs: 57-60. In some embodiments, the nucleic acid molecule comprises a nucleic acid sequence that is at least 70% identical to any one of SEQ ID NOs: 57-60. In some embodiments, the nucleic acid molecule comprises a nucleic acid sequence that is at least 80% identical to any one of SEQ ID NOs: 57-60. In some embodiments, the nucleic acid molecule comprises a nucleic acid sequence that is at least 90% identical to any one of SEQ ID NOs: 57-60.

In accordance with these embodiments, the nucleic acid molecule comprises a K_{D} that is less than about 2500 nM. In some embodiments, the nucleic acid molecule comprises a K_{D} that is less than about 2000 nM. In some embodiments, the nucleic acid molecule comprises a K_{D} that is less than about 1500 nM. In some embodiments, the nucleic acid molecule comprises a K_{D} that is less than about 1000 mM. In some embodiments, the nucleic acid molecule comprises a K_{D} that is less than about 900 nM. In some embodiments, the nucleic acid molecule comprises a K_{D} that is less than about 800 nM. In some embodiments, the nucleic acid molecule comprises a K_{D} that is less than about 700 nM. In some embodiments, the nucleic acid molecule comprises a K_{D} that is less than about 600 nM. In some embodiments, the nucleic acid molecule comprises a K_{D} that is less than about 500 nM. In some embodiments, the nucleic acid molecule comprises a K_{D} that is less than about 400 nM. In some embodiments, the nucleic acid molecule comprises a K_{D} that is less than about 300 nM. In some embodiments, the nucleic acid molecule comprises a K_{D} that is less than about 200 nM. In some embodiments, the nucleic acid molecule comprises a K_{D} that is less than about 100 nM. In some embodiments, the nucleic acid molecule comprises a K_{D} that is less than about 50 nM. In some embodiments, the nucleic acid molecule comprises a K_{D} that is less than about 25 nM.

Embodiments of the present disclosure also include a single-stranded nucleic acid molecule capable of specifically binding oxycodone or oxymorphone, or a derivative or analog thereof, comprising a nucleic acid sequence that is at least 50% identical to the following: ATGGGAT(X)₁₋₄ATGTGGTGT; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; and X₁₄ is A, T, C, or G (SEQ ID NO: 42). In some embodiments, the nucleic acid is that is at least 60% identical to the following: ATGGGAT(X)₁₋₄ATGTGGTGT; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; and X₁₄ is A, T, C, or G (SEQ ID NO: 42). In some embodiments, the nucleic acid is that is at least 70% identical to the following: ATGGGAT(X)₁₋₄ATGTGGTGT; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; and X₁₄ is A, T, C, or G (SEQ ID NO: 42). In some embodiments, the nucleic acid is that is at least 80% identical to the following: ATGGGAT(X)₁₋₄ATGTGGTGT; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; and X₁₄ is A, T, C, or G (SEQ ID NO: 42). In some embodiments, the nucleic acid is that is at least 90% identical to the following: ATGGGAT(X)₁₋₄ATGTGGTGT; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; and X₁₄ is A, T, C, or G (SEQ ID NO: 42).

In some embodiments, the nucleic sequence is from about 50% to about 90% identical to the following: ATGGGAT(X)₁₋₄ATGTGGTGT; wherein X₁ is A or G; X₂ is C or G; X₃ is G, A, or C; X₄ is A or G; X₅ is A, C, or G; X₆ is C, T, or A; X₇ is T, A, or G; X₈ is C or T; X₉ is G or C; X₁₀ is T or C; X₁₁ is T or G; X₁₂ is T, A, or G; X₁₃ is G; and X₁₄ is G or C (SEQ ID NO: 43).

In some embodiments, the nucleic acid molecule comprises a nucleic acid sequence that is at least 50% identical to any one of SEQ ID NOs: 61-63. In some embodiments, the nucleic acid molecule comprises a nucleic acid sequence that is at least 60% identical to any one of SEQ ID NOs: 61-63. In some embodiments, the nucleic acid molecule comprises a nucleic acid sequence that is at least 70% identical to any one of SEQ ID NOs: 61-63. In some embodiments, the nucleic acid molecule comprises a nucleic acid sequence that is at least 80% identical to any one of SEQ ID NOs: 61-63. In some embodiments, the nucleic acid molecule comprises a nucleic acid sequence that is at least 90% identical to any one of SEQ ID NOs: 61-63.

In accordance with these embodiments, the nucleic acid molecule comprises a K_{D} that is less than about 3500 nM. In some embodiments, the nucleic acid molecule comprises a K_{D} that is less than about 3000 nM. In some embodiments, the nucleic acid molecule comprises a K_{D} that is less than about 2500 nM. In some embodiments, the nucleic acid molecule comprises a K_{D} that is less than about 2000 nM. In some embodiments, the nucleic acid molecule comprises a K_{D} that is less than about 1500 nM. In some embodiments, the nucleic acid molecule comprises a K_{D} that is less than about 1000 nM. In some embodiments, the nucleic acid molecule comprises a K_{D} that is less than about 900 nM. In some embodiments, the nucleic acid molecule comprises a K_{D} that is less than about 800 nM. In some embodiments, the nucleic acid molecule comprises a K_{D} that is less than about 700 nM. In some embodiments, the nucleic acid molecule comprises a K_{D} that is less than about 600 nM. In some embodiments, the nucleic acid molecule comprises a K_{D} that is less than about 500 nM. In some embodiments, the nucleic acid molecule comprises a K_{D} that is less than about 400 nM. In some embodiments, the nucleic acid molecule comprises a K_{D} that is less than about 300 nM. In some embodiments, the nucleic acid molecule comprises a K_{D} that is less than about 200 nM. In some embodiments, the nucleic acid molecule comprises a K_{D} that is less than about 100 nM. In some embodiments, the nucleic acid molecule comprises a K_{D} that is less than about 50 nM. In some embodiments, the nucleic acid molecule comprises a K_{D} that is less than about 25 nM.

Embodiments of the present disclosure also include a single-stranded nucleic acid molecule capable of specifically binding oxycodone or oxymorphone, or a derivative or analog thereof, comprising a nucleic acid sequence that is at least 50% identical to the following: (X)₃₋₄ATGTGGTGT(X)₇₋₈ATGGGAT(X)₃; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; and X₁₅ is A, T, C, or G (SEQ ID NO: 44). In some embodiments, X₁ is G; X₂ is C or G; X₃ is G; X₄ is C; X₅ is C; X₆ is G or A; X₇ is G; X₈ is T; X₉ is C; X₁₀ is A; X₁₁ is G or T; X₁₂ is G; X₁₃ is G or C; X₁₄ is A; and X₁₅ is A (SEQ ID NO: 45). In some embodiments, the nucleic acid molecule comprises a nucleic acid sequence that is at least 50% identical to SEQ ID NOs: 64 or 65. In some embodiments, the nucleic acid molecule comprises a nucleic acid sequence that is at least 60% identical to SEQ ID NOs: 64 or 65. In some embodiments, the nucleic acid molecule comprises a nucleic acid sequence that is at least 70% identical to SEQ ID NOs: 64 or 65. In some embodiments, the nucleic acid molecule comprises a nucleic acid sequence that is at least 80% identical to SEQ ID NOs: 64 or 65. In some embodiments, the nucleic acid molecule comprises a nucleic acid sequence that is at least 90% identical to SEQ ID NOs: 64 or 65. In accordance with these embodiments, (X)₃₋₄ indicates that X₃ is present in the nucleic acid molecule, but X₄ is optional. Additionally, (X)₇₋₈ indicates that X₇ is present in the nucleic acid molecule, but X₈ is optional.

In accordance with these embodiments, the nucleic acid molecule comprises a K_{D} that is less than about 4000 nM. In some embodiments, the nucleic acid molecule comprises a K_{D} that is less than about 3500 nM. In some embodiments, the nucleic acid molecule comprises a K_{D} that is less than about 3000 nM. In some embodiments, the nucleic acid molecule comprises a K_{D} that is less than about 2500 nM. In some embodiments, the nucleic acid molecule comprises a K_{D} that is less than about 2000 nM. In some embodiments, the nucleic acid molecule comprises a K_{D} that is less than about 1500 nM. In some embodiments, the nucleic acid molecule comprises a K_{D} that is less than about 1000 nM. In some embodiments, the nucleic acid molecule comprises a K_{D} that is less than about 900 nM. In some embodiments, the nucleic acid molecule comprises a K_{D} that is less than about 800 nM. In some embodiments, the nucleic acid molecule comprises a K_{D} that is less than about 700 nM. In some embodiments, the nucleic acid molecule comprises a K_{D} that is less than about 600 nM. In some embodiments, the nucleic acid molecule comprises a K_{D} that is less than about 500 nM. In some embodiments, the nucleic acid molecule comprises a K_{D} that is less than about 400 nM. In some embodiments, the nucleic acid molecule comprises a K_{D} that is less than about 300 nM. In some embodiments, the nucleic acid molecule comprises a K_{D} that is less than about 200 nM. In some embodiments, the nucleic acid molecule comprises a K_{D} that is less than about 100 nM. In some embodiments, the nucleic acid molecule comprises a K_{D} that is less than about 50 nM. In some embodiments, the nucleic acid molecule comprises a K_{D} that is less than about 25 nM.

Embodiments of the present disclosure also include a single-stranded nucleic acid molecule capable of specifically binding oxycodone or oxymorphone, or a derivative or analog thereof, comprising a nucleic acid sequence that is at least 50% identical to any one of SEQ ID NOs: 66-70. In some embodiments, the nucleic acid is at least 60% identical to any one of SEQ ID NOs: 66-70. In some embodiments, the nucleic acid is at least 70% identical to any one of SEQ ID NOs: 66-70. In some embodiments, the nucleic acid is at least 80% identical to any one of SEQ ID NOs: 66-70. In some embodiments, the nucleic acid is at least 90% identical to any one of SEQ ID NOs: 66-70.

In some embodiments, the nucleic acid molecule comprises a detection moiety. In some embodiments, the detection moiety is a florescent dye, an antibody, a probe, an enzyme, a ligand, a nanoparticle, a radioactive isotope, a chemiluminescent substrate, a click chemistry reagent, a biotin conjugated molecule or a derivative or analog thereof. In some embodiments, the nucleic acid molecule is in solution or attached to a substrate.

Embodiments of the present disclosure also include a vector comprising any of the nucleic acid sequences of the aptamers described herein.

### 3. METHODS OF USE

Embodiments of the present disclosure also include a method of detecting an opioid, or a derivative or analog thereof. In accordance with these embodiments, the methods include combining any of the nucleic acid molecules of the aptamers described herein to a fluorescent moiety with a quencher-labeled nucleic acid molecule that is at least partially complementary to the nucleic acid molecules of the aptamers described herein to form a quenched composition; and exposing the quenched composition to a sample comprising or suspected of comprising an opioid, or a derivative or analog thereof. In some embodiments, the presence of the opioid, or a derivative or analog thereof, in the sample displaces the quencher-labeled nucleic acid molecule, thereby producing a fluorescent signal proportional to the concentration of the opioid, or a derivative or analog thereof, in the sample.

In other embodiments, a method of detecting opioid, or a derivative or analog thereof. In accordance with these embodiments, the methods include combining any of the nucleic acid molecules of the aptamers described herein with a reporter compound that binds to the nucleic acid molecules of the aptamers described herein to form a complexed composition; and exposing the complexed composition to a sample comprising or suspected of comprising an opioid, or a derivative or analog thereof. In some embodiments, the presence of the opioid, or a derivative or analog thereof, in the sample displaces the reporter compound, thereby allowing the reporter compound to form detectable aggregates proportional to the concentration of the opioid, or a derivative or analog thereof, in the sample.

In other embodiments, a method of detecting opioid, or a derivative or analog thereof. In accordance with these embodiments, the methods include immobilizing any of the nucleic acid molecules of the aptamers described herein to an electrically conductive substrate, wherein the nucleic acid molecules of the aptamers described herein comprising a redox tag, to form a detection sensor; and exposing the detection sensor to a sample comprising or suspected of comprising an opioid, or a derivative or analog thereof. In some embodiments, the presence of the opioid, or a derivative or analog thereof, in the sample binds the nucleic acid molecules of the aptamers described herein, thereby producing an electrochemical signal proportional to the concentration of the opioid, or a derivative or analog thereof, in the sample.

In some embodiments, the sample is a biological sample from a human subject. In some embodiments, the biological sample is a saliva sample, a urine sample, a blood sample, a serum sample, a plasma sample, a fecal sample, a CSF sample, or a tissue sample.

In some embodiments of these methods, the opioid derivative or analog comprised of: Dihydrocodeine, Oxymorphone, Hydromorphone, Levorphanol, Meperidine, Dextropropoxyphene, Butorphanol, Pentazocine, Nalbuphine, Tapentadol, AH-7921, U-47700, MT-45, Acetyl fentanyl, Furanyl fentanyl, Desomorphine, Carfentanil, Sufentanil, Remifentanil, Alfentanil, Butyrfentanyl, Valeryl fentanyl, Para-Methoxyfentanyl, Cyclopropyl fentanyl, Cyclopentyl fentanyl, Isobutyryl fentanyl, Methoxyacetyl fentanyl, 3-Methylfentanyl, 3-Methylthiofentanyl, 4-Fluoroisobutyrfentanyl, Hydrocodone, Oxycodone, Buprenorphine, Codeine, Fentanyl, Methadone.

### 4. MATERIALS AND METHODS

Reagents and Materials. Molecular biology grade water was purchased from Corning. Ultrapure water with a resistivity of 18.2 MΩ·cm was obtained from a Milli-Q EQ7000 water purification system. Exonuclease I (Exo I, E. coli; 20 U/μl), and T5 exonuclease (T5 Exo; 10 U/μl) were purchased from New England Biolabs. Morphine sulfate hydrate, codeine phosphate hydrate, heroin HCl, oxycodone HCl, oxymorphone HCl, hydrocodone bitartate, hydromorphone HCl, acetyl fentanyl HCl, fentanyl HCl, diazepam, alprazolam, clonazepam, (+)-methamphetamine HCl, ethylone HCl polymorph B and methylnaltrexone bromide was purchased from Cayman Chemicals. Acetaminophen, benzocaine HCl, caffeine, cocaine HCl, chlorpromazine HCl, diphenhydramine HCl, lactose, mannitol, lidocaine HCl, naloxone HCl, naltrexone HCl, quinine hemisulfate monohydrate, and sodium dodecyl sulfate were purchased from Sigma-Aldrich. Noscapine HCl was purchased from Tokyo Chemical Industry. Papaverine HCl was purchased from Acros Organics. Levamisole HCl and xylazine HCl were purchased from MP Biomedicals. GoTaq Hot Start Master Mix was purchased from Promega. QIAquick PCR purification kit was purchased from Qiagen. SYBR Gold, streptavidin-coated agarose resin (capacity: 1-3 mg biotinylated BSA/ml resin), 0.5 M EDTA solution (pH 8.0), and formamide were purchased from Thermo Fisher Scientific. All other chemicals were purchased from Sigma-Aldrich unless otherwise specified.

Oligonucleotides. DNA oligonucleotides were purchased from Integrated DNA Technologies with standard desalting purification. The random library, primers, and a 15-nt biotinylated complementary DNA (cDNA15-bio) were HPLC purified. DNA was dissolved in molecular biology grade water and their concentrations were determined using a NanoDrop 2000 Spectrophotometer (Thermo Fisher Scientific). The oligonucleotide sequences in this work are shown in Table 1 below.

TABLE 1
Sequences of library, cDNA, primers employed during SELEX.

| Seq. ID | Sequence (5′ to 3′) |
|---|---|
| Library | GGAGGCTCTCGGGACGAC(N30)GTCGTCCCGCCTTTAGGATTTACAG (SEQ ID NO: 71) |
| cDNA15-bio | GTCGTCCCGAGAGCCATA/3BioTEG/ (SEQ ID NO: 72) |
| FP | GGAGGCTCTCGGGACGAC (SEQ ID NO: 73) |
| RP-bio | /5Biosg/CTGTAAATCCTAAAGGCGGGACGAC (SEQ ID NO: 74) |
| FP-HTS | ACACTCTTTCCCTACACGACGCTCTTCCGATCT(N45)GGAGGCTCTCGGGACGAC (SEQ |
| | ID NO: 75) |
| RP-HTS | GACTGGAGTTCAGACGTGTGCTCTTCCGATCT(N43)CTGTAAATCCTAAAGGCGG |
| | GACGAC (SEQ ID NO: 76) |

/3BioTEG/ and /5Biosg/ = 3′ and 5′ biotin modification, respectively.

Buffers. All experiments were performed in the selection buffer (10 mM Tris-HCl (pH 7.4), 20 mM NaCl, 0.5 mM MgCl₂). For dye displacement experiments, the selection buffer included 0.01% SDS as a surfactant. Quenching solution for exonuclease digestion assays contained 10 mM Tris-HCl (pH 7.4), 21 mM EDTA, 12.5% (v/v) formamide, and 1×SYBR Gold (final concentrations).

Library-immobilized selection for isolating morphinan-based opioid-binding aptamers. A library-immobilized selection strategy was employed as previously described. For each round, the library was mixed with a 5-fold excess of biotinylated 15-nt cDNA in selection buffer, heated to 90° C. for 10 min, and slowly cooled to room temperature over 20 min in a water bath to promote annealing of both strands. The library-cDNA complex was then immobilized onto streptavidin-coated agarose beads loaded in a gravity column (0.8 mL) and pre-conditioned with selection buffer. The library-bead assembly was washed several times with the selection buffer to remove non-specifically eluting library strands. Counter-SELEX was performed from Round 2 onward to remove aptamers that bind to interferents. Afterwards, positive selection with the target was performed by adding the target to the column and collecting all eluted library molecules. These eluted strands were PCR amplified under the following reaction conditions: 2 min at 95° C.; 11 cycles of 95° C. for 15 s, 58° C. for 30 s, and 72° C. for 45 s; and finally, 5 min at 72° C. Successful PCR amplification of the library was confirmed using agarose gel electrophoresis. The amplicons were then converted to single-stranded DNA using streptavidin agarose resin and NaOH treatment. Finally, the pool was subjected to another round of SELEX. Pool affinity and specificity were periodically assessed using a previously reported gel-elution assay.

Parallel-and-serial selection was employed to isolate cross-reactive aptamers for heroin and morphine. Individual pools for heroin or morphine were enriched for six rounds, after which a gel elution assay was performed to confirm target binding. To perform serial selection, 150 pmol each of the Round 6 heroin and morphine pools were mixed and challenged with heroin for one round, followed by morphine in the next round. This selection was then repeated for Rounds 3 and 4 of serial selection. A gel elution assay was used to determine the binding affinity and specificity of the final pool. Details about the selection conditions can be found in Tables 2-4 below.

TABLE 2
Selection strategy and experimental conditions for heroin.

| | Pool size | Buffer | | Buffer | [Heroin] |
|---|---|---|---|---|---|
| Round | (pmole) | Wash | Counter-targets | Wash | (μM) |
| 1 | 1000 | 10 | NA | NA | 500 |
| 2 | 419 | 10 | 200 μM PAP (250 μl x10), 200 μM NOS (250 μl x10), 200 μM DIA (250 μl x10), | 30 | 500 |
| | | | 200 μM G1 (250 μl x3), 200 μM G2 (250 μl x3), 200 μM G3 (250 μl x3), 200 μM | | |
| | | | NOL (250 μl x3), 200 μM NAL (250 μl x3), 200 μM mNAL (250 μl x3) | | |
| 3 | 301 | 10 | 200 μM PAP (250 μl x10), 200 μM NOS (250 μl x10), 200 μM DIA (250 μl x10), | 30 | 500 |
| | | | 200 μM G1 (250 μl x3), 200 μM G2 (250 μl x3), 200 μM G3 (250 μl x3), 200 μM | | |
| | | | NOL (250 μl x3), 200 μM NAL (250 μl x3), 200 μM mNAL (250 μl x3) | | |
| 4 | 300 | 10 | 200 μM PAP (250 μl x10), 200 μM NOS (250 μl x10), 200 μM DIA (250 μl x10), | 30 | 500 |
| | | | 300 μM G1 (250 μl x3), 300 μM G2 (250 μl x3), 300 μM G3 (250 μl x3), 300 μM | | |
| | | | NOL (250 μl x3), 300 μM NAL (250 μl x3), 300 μM mNAL (250 μl x3) | | |
| 5 | 300 | 10 | 200 μM PAP (250 μl x10), 200 μM NOS (250 μl x10), 200 μM DIA (250 μl x10), | 30 | 500 |
| | | | 400 μM G1 (250 μl x3), 400 μM G2 (250 μl x3), 400 μM G3 (250 μl x3), 400 μM | | |
| | | | NOL (250 μl x3), 400 μM NAL (250 μl x3), 400 μM mNAL (250 μl x3) | | |
| 6 | 300 | 10 | 200 μM PAP (250 μl x10), 200 μM NOS (250 μl x10), 200 μM DIA (250 μl x10), | 30 | 250 |
| | | | 500 μM G1 (250 μl x3), 500 μM G2 (250 μl x3), 500 μM G3 (250 μl x3), 500 μM | | |
| | | | NOL (250 μl x3), 500 μM NAL (250 μl x3), 500 μM mNAL (250 μl x3), 500 μM | | |
| | | | AF (250 μl x3), 500 μM G4 (250 μl x3), 200 μM G5 (250 μl x3), 500 μM G6 | | |
| | | | (250 μl x3), | | |

Counter-target abbreviations: Papaverine (PAP), noscapine (NOS), diazepam (DIA), procaine (PRO), quinine (QUI), chlorpromazine (CPZ), cocaine (COC), lidocaine (LID), benzocaine (BZC), diphenhydramine (DPH), levamisole (LEV), acetaminophen (ACM), caffeine (CAF), mannitol (MAN), lactose, (LAC), xylazine (XYL), clonazepam (CLO), alprazolam (ALP), (+)-methamphetamine (METH), (+)-pseudoephedrine (PSU), ethylone (ETY), G1 (combination of PRO, QUI, & CPZ), G2 (combination of COC, LID, & BZC), G3 (combination of DPH, LEV, & ACM), G4 (combination of CAF, MAN, LAC, & XYL), G5 (combination of CLO & ALP), and G6 (combination of METH, PSU, & ETY).

TABLE 3
Selection strategy and experimental conditions for morphine.

| | Pool size | Buffer | | Buffer | [Morphine] |
|---|---|---|---|---|---|
| Round | (pmole) | Wash | Counter-targets | Wash | (μM) |
| 1 | 1000 | 10 | NA | NA | 500 |
| 2 | 390 | 10 | 200 μM PAP (250 μl x10), 200 μM NOS (250 μl x10), 200 μM DIA (250 μl x10), | 30 | 500 |
| | | | 200 μM G1 (250 μl x3), 200 μM G2 (250 μl x3), 200 μM G3 (250 μl x3), 200 μM | | |
| | | | NOL (250 μl x3), 200 μM NAL (250 μl x3), 200 μM mNAL (250 μl x3) | | |
| 3 | 352 | 10 | 200 μM PAP (250 μl x10), 200 μM NOS (250 μl x10), 200 μM DIA (250 μl x10), | 30 | 500 |
| | | | 200 μM G1 (250 μl x3), 200 μM G2 (250 μl x3), 200 μM G3 (250 μl x3), 200 μM | | |
| | | | NOL (250 μl x3), 200 μM NAL (250 μl x3), 200 μM mNAL (250 μl x3) | | |
| 4 | 300 | 10 | 200 μM PAP (250 μl x10), 200 μM NOS (250 μl x10), 200 μM DIA (250 μl x10), | 30 | 500 |
| | | | 300 μM G1 (250 μl x3), 300 μM G2 (250 μl x3), 300 μM G3 (250 μl x3), 300 μM | | |
| | | | NOL (250 μl x3), 300 μM NAL (250 μl x3), 300 μM mNAL (250 μl x3) | | |
| 5 | 300 | 10 | 200 μM PAP (250 μl x10), 200 μM NOS (250 μl x10), 200 μM DIA (250 μl x10), | 30 | 500 |
| | | | 400 μM G1 (250 μl x3), 400 μM G2 (250 μl x3), 400 μM G3 (250 μl x3), 400 μM | | |
| | | | NOL (250 μl x3), 400 μM NAL (250 μl x3), 400 μM mNAL (250 μl x3) | | |
| 6 | 300 | 10 | 200 μM PAP (250 μl x10), 200 μM NOS (250 μl x10), 200 μM DIA (250 μl x10), | 30 | 250 |
| | | | 500 μM G1 (250 μl x3), 500 μM G2 (250 μl x3), 500 μM G3 (250 μl x3), 500 μM | | |
| | | | NOL (250 μl x3), 500 μM NAL (250 μl x3), 500 μM mNAL (250 μl x3), 500 μM | | |
| | | | AF (250 μl x3), 500 μM G4 (250 μl x3), 200 μM G5 (250 μl x3), 500 μM G6 | | |
| | | | (250 μl x3), | | |

Counter-target abbreviations: Papaverine (PAP), noscapine (NOS), diazepam (DIA), procaine (PRO), quinine (QUI), chlorpromazine (CPZ), cocaine (COC), lidocaine (LID), benzocaine (BZC), diphenhydramine (DPH), levamisole (LEV), acetaminophen (ACM), caffeine (CAF), mannitol (MAN), lactose, (LAC), xylazine (XYL), clonazepam (CLO), alprazolam (ALP), (+)-methamphetamine (METH), (+)-pseudoephedrine (PSU), ethylone (ETY), G1 (combination of PRO, QUI, & CPZ), G2 (combination of COC, LID, & BZC), G3 (combination of DPH, LEV, & ACM), G4 (combination of CAF, MAN, LAC, & XYL), G5 (combination of CLO & ALP), and G6 (combination of METH, PSU, & ETY).

TABLE 4
Selection strategy and conditions for heroin and morphine serial selection.

| | Pool size | Buffer | | Buffer | [Target] |
|---|---|---|---|---|---|
| Round | (pmole) | Wash | Counter-targets | Wash | (μM) |
| S1 | 150 Heroin R6 and | 10 | 100 μM PAP (250 μl x10), 100 μM NOS (250 μl x10), 100 μM DIA (250 μl | 30 | 50 |
| | 150 Morphine R6 | | x10), 250 μM QUI (250 μl x3), 250 μM CPZ (250 μl x3), 1000 μM PRO (250 μl | | Heroin |
| | | | x3), 1000 μM COC (250 μl x3), 1000 μM LID (250 μl x3), 1000 μM BZC | | |
| | | | (250 μl x3), 1000 μM DPH (250 μl x3), 1000 μM LEV (250 μl x3), 1000 μM | | |
| | | | ACM (250 μl x3), 1000 μM NOL (250 μl x3), 1000 μM NAL (250 μl x3), | | |
| | | | 1000 μM mNAL (250 μl x3), 1000 μM CAF (250 μl x3), 1000 μM MAN (250 μl | | |
| | | | x3), 1000 μM LAC (250 μl x3), 1000 μM XYL (250 μl x3), 200 μM AF | | |
| | | | (250 μl x3), 100 μM CLO (250 μl x3), 100 μM ALP (250 μl x3), 1000 μM | | |
| | | | METH (250 μl x3), 1000 μM PSU (250 μl x3), 1000 μM ETY (250 μl x3), | | |
| S2 | 300 | 10 | 100 μM PAP (250 μl x10), 100 μM NOS (250 μl x10), 100 μM DIA (250 μl | 30 | 50 |
| | | | x10), 250 μM QUI (250 μl x3), 250 μM CPZ (250 μl x3), 1000 μM PRO (250 μl | | Morphine |
| | | | x3), 1000 μM COC (250 μl x3), 1000 μM LID (250 μl x3), 1000 μM BZC | | |
| | | | (250 μl x3), 1000 μM DPH (250 μl x3), 1000 μM LEV (250 μl x3), 1000 μM | | |
| | | | ACM (250 μl x3), 1000 μM NOL (250 μl x3), 1000 μM NAL (250 μl x3), | | |
| | | | 1000 μM mNAL (250 μl x3), 1000 μM CAF (250 μl x3), 1000 μM MAN (250 μl | | |
| | | | x3), 1000 μM LAC (250 μl x3), 1000 μM XYL (250 μl x3), 200 μM AF | | |
| | | | (250 μl x3), 100 μM CLO (250 μl x3), 100 μM ALP (250 μl x3), 1000 μM | | |
| | | | METH (250 μl x3), 1000 μM PSU (250 μl x3), 1000 μM ETY (250 μl x3), | | |
| S3 | 300 | 10 | 100 μM PAP (250 μl x10), 100 μM NOS (250 μl x10), 100 μM DIA (250 μl | 30 | 25 |
| | | | x10), 250 μM QUI (250 μl x3), 250 μM CPZ (250 μl x3), 1000 μM PRO (250 μl | | Heroin |
| | | | x3), 1000 μM COC (250 μl x3), 1000 μM LID (250 μl x3), 1000 μM BZC | | |
| | | | (250 μl x3), 1000 μM DPH (250 μl x3), 1000 μM LEV (250 μl x3), 1000 μM | | |
| | | | ACM (250 μl x3), 1000 μM NOL (250 μl x3), 1000 μM NAL (250 μl x3), | | |
| | | | 1000 μM mNAL (250 μl x3), 1000 μM CAF (250 μl x3), 1000 μM MAN (250 μl | | |
| | | | x3), 1000 μM LAC (250 μl x3), 1000 μM XYL (250 μl x3), 200 μM AF | | |
| | | | (250 μl x3), 100 μM CLO (250 μl x3), 100 μM ALP (250 μl x3), 1000 μM | | |
| | | | METH (250 μl x3), 1000 μM PSU (250 μl x3), 1000 μM ETY (250 μl x3), | | |
| S4 | 300 | 10 | 100 μM PAP (250 μl x10), 100 μM NOS (250 μl x10), 100 μM DIA (250 μl | 30 | 25 |
| | | | x10), 250 μM QUI (250 μl x3), 250 μM CPZ (250 μl x3), 1000 μM PRO (250 μl | | Morphine |
| | | | x3), 1000 μM COC (250 μl x3), 1000 μM LID (250 μl x3), 1000 μM BZC | | |
| | | | (250 μl x3), 1000 μM DPH (250 μl x3), 1000 μM LEV (250 μl x3), 1000 μM | | |
| | | | ACM (250 μl x3), 1000 μM NOL (250 μl x3), 1000 μM NAL (250 μl x3), | | |
| | | | 1000 μM mNAL (250 μl x3), 1000 μM CAF (250 μl x3), 1000 μM MAN (250 μl | | |
| | | | x3), 1000 μM LAC (250 μl x3), 1000 μM XYL (250 μl x3), 200 μM AF | | |
| | | | (250 μl x3), 100 μM CLO (250 μl x3), 100 μM ALP (250 μl x3), 1000 μM | | |
| | | | METH (250 μl x3), 1000 μM PSU (250 μl x3), 1000 μM ETY (250 μl x3), | | |

Counter-target abbreviations: Papaverine (PAP), noscapine (NOS), diazepam (DIA), procaine (PRO), quinine (QUI), chlorpromazine (CPZ), cocaine (COC), lidocaine (LID), benzocaine (BZC), diphenhydramine (DPH), levamisole (LEV), acetaminophen (ACM), caffeine (CAF), mannitol (MAN), lactose, (LAC), xylazine (XYL), clonazepam (CLO), alprazolam (ALP), (+)-methamphetamine (METH), (+)-pseudoephedrine (PSU), ethylone (ETY), G1 (combination of PRO, QUI, & CPZ), G2 (combination of COC, LID, & BZC), G3 (combination of DPH, LEV, & ACM), G4 (combination of CAF, MAN, LAC, & XYL), G5 (combination of CLO & ALP), and G6 (combination of METH, PSU, & ETY).

For the isolation of cross-reactive aptamers for oxycodone and oxymorphone, a pool was first enriched using oxycodone as the target for six rounds. Then, a toggle selection strategy was applied, such that in the seventh round, oxymorphone was used as the selection target, followed by oxycodone during the eighth round. The selection target was subsequently toggled between oxycodone or oxymorphone for four additional rounds, after which the pool affinity and specificity were confirmed using a gel elution assay. Details about selection conditions can be found in Tables 5-6 below.

TABLE 5
Selection strategy and conditions for oxycodone.

| | Pool size | Buffer | | Buffer | [Oxycodone] |
|---|---|---|---|---|---|
| Round | (pmole) | Wash | Counter-targets | Wash | (μM) |
| 1 | 1000 | 10 | NA | NA | 500 |
| 2 | 400 | 10 | 100 μM PAP (250 μl x10), 100 μM NOS (250 μl x10), 100 μM DIA (250 μl x10), | 30 | 500 |
| | | | 200 μM QUI (250 μl x3), 200 μM PRO (250 μl x3), 200 μM CPZ (250 μl x3), 200 μM | | |
| | | | G2 (250 μl x3), 200 μM G3 (250 μl x3), 200 μM NOL (250 μl x3), 200 μM | | |
| | | | NAL (250 μl x3), 200 μM mNAL (250 μl x3), 200 μM AF (250 μl x3), 100 μM | | |
| | | | CLO (250 μl x3), 100 μM ALP (250 μl x3) | | |
| 3 | 386 | 10 | 100 μM PAP (250 μl x10), 100 μM NOS (250 μl x10), 100 μM DIA (250 μl x10), | 30 | 500 |
| | | | 250 μM QUI (250 μl x3), 300 μM PRO (250 μl x3), 250 μM CPZ (250 μl x3), 300 μM | | |
| | | | G2 (250 μl x3), 300 μM G3 (250 μl x3), 300 μM NOL (250 μl x3), 300 μM | | |
| | | | NAL (250 μl x3), 300 μM mNAL (250 μl x3), 300 μM AF (250 μl x3), 100 μM | | |
| | | | CLO (250 μl x3), 100 μM ALP (250 μl x3) | | |
| 4 | 300 | 10 | 100 μM PAP (250 μl x10), 100 μM NOS (250 μl x10), 100 μM DIA (250 μl x10), | 30 | 500 |
| | | | 250 μM QUI (250 μl x3), 400 μM PRO (250 μl x3), 250 μM CPZ (250 μl x3), 400 μM | | |
| | | | G2 (250 μl x3), 400 μM G3 (250 μl x3), 400 μM NOL (250 μl x3), 400 μM | | |
| | | | NAL (250 μl x3), 400 μM mNAL (250 μl x3), 400 μM AF (250 μl x3), 100 μM | | |
| | | | CLO (250 μl x3), 100 μM ALP (250 μl x3) | | |
| 5 | 300 | 10 | 100 μM PAP (250 μl x10), 100 μM NOS (250 μl x10), 100 μM DIA (250 μl x10), | 30 | 500 |
| | | | 250 μM QUI (250 μl x3), 500 μM PRO (250 μl x3), 250 μM CPZ (250 μl x3), 500 μM | | |
| | | | G2 (250 μl x3), 500 μM G3 (250 μl x3), 500 μM NOL (250 μl x3), 500 μM | | |
| | | | NAL (250 μl x3), 500 μM mNAL (250 μl x3), 500 μM G4 (250 μl x3), 500 μM AF | | |
| | | | (250 μl x3), 100 μM CLO (250 μl x3), 100 μM ALP (250 μl x3), 500 μM G6 | | |
| | | | (250 μl x3), | | |
| 6 | 300 | 10 | 100 μM PAP (250 μl x10), 100 μM NOS (250 μl x10), 100 μM DIA (250 μl x10), | 30 | 250 |
| | | | 250 μM QUI (250 μl x3), 500 μM PRO (250 μl x3), 250 μM CPZ (250 μl x3), 500 μM | | |
| | | | G2 (250 μl x3), 500 μM G3 (250 μl x3), 500 μM NOL (250 μl x3), 500 μM | | |
| | | | NAL (250 μl x3), 500 μM mNAL (250 μl x3), 500 μM G4 (250 μl x3), 500 μM AF | | |
| | | | (250 μl x3), 100 μM CLO (250 μl x3), 100 μM ALP (250 μl x3), 500 μM G6 | | |
| | | | (250 μl x3), | | |

Counter target abbreviations: Papaverine (PAP), noscapine (NOS), diazepam (DIA), procaine (PRO), quinine (QUI), chlorpromazine (CPZ), cocaine (COC), lidocaine (LID), benzocaine (BZC), diphenhydramine (DPH), levamisole (LEV), acetaminophen (ACM), caffeine (CAF), mannitol (MAN), lactose, (LAC), xylazine (XYL), clonazepam (CLO), alprazolam (ALP), (+)-methamphetamine (METH), (+)-pseudoephedrine (PSU), ethylone (ETY), G1 (combination of PRO, QUI, & CPZ), G2 (combination of COC, LID, & BZC), G3 (combination of DPH, LEV, & ACM), G4 (combination of CAF, MAN, LAC, & XYL), G5 (combination of CLO & ALP), and G6 (combination of METH, PSU, & ETY).

TABLE 6
Selection strategy and conditions of toggle selection for oxycodone and oxymorphone.

| | Pool size | Buffer | | Buffer | [Target] |
|---|---|---|---|---|---|
| Round | (pmole) | Wash | Counter-targets | Wash | (μM) |
| T1 | 300 R6 | 10 | 100 μM PAP (250 μl x10), 100 μM NOS (250 μl x10), 100 μM DIA (250 μl | 30 | 100 |
| | Oxycodone | | x10), 250 μM QUI (250 μl x3), 250 μM CPZ (250 μl x3), 1000 μM PRO (250 μl | | Oxymorphone |
| | | | x3), 1000 μM COC (250 μl x3), 1000 μM LID (250 μl x3), 1000 μM BZC | | |
| | | | (250 μl x3), 1000 μM DPH (250 μl x3), 1000 μM LEV (250 μl x3), 1000 μM | | |
| | | | ACM (250 μl x3), 1000 μM NOL (250 μl x10), 1000 μM NAL (250 μl x10), | | |
| | | | 1000 μM mNAL (250 μl x10), 1000 μM CAF (250 μl x3), 1000 μM MAN | | |
| | | | (250 μl x3), 1000 μM LAC (250 μl x3), 1000 μM XYL (250 μl x3), 200 μM | | |
| | | | AF (250 μl x3), 100 μM CLO (250 μl x3), 100 μM ALP (250 μl x3), 1000 μM | | |
| | | | METH (250 μl x3), 1000 μM PSU (250 μl x3), 1000 μM ETY (250 μl x3), | | |
| T2 | 300 | 10 | 100 μM PAP (250 μl x10), 100 μM NOS (250 μl x10), 100 μM DIA (250 μl | 30 | 100 |
| | | | x10), 250 μM QUI (250 μl x3), 250 μM CPZ (250 μl x3), 1000 μM PRO (250 μl | | Oxycodone |
| | | | x3), 1000 μM COC (250 μl x3), 1000 μM LID (250 μl x3), 1000 μM BZC | | |
| | | | (250 μl x3), 1000 μM DPH (250 μl x3), 1000 μM LEV (250 μl x3), 1000 μM | | |
| | | | ACM (250 μl x3), 1000 μM NOL (250 μl x10), 1000 μM NAL (250 μl x10), | | |
| | | | 1000 μM mNAL (250 μl x10), 1000 μM CAF (250 μl x3), 1000 μM MAN | | |
| | | | (250 μl x3), 1000 μM LAC (250 μl x3), 1000 μM XYL (250 μl x3), 200 μM | | |
| | | | AF (250 μl x3), 100 μM CLO (250 μl x3), 100 μM ALP (250 μl x3), 1000 μM | | |
| | | | METH (250 μl x3), 1000 μM PSU (250 μl x3), 1000 μM ETY (250 μl x3), | | |
| T3 | 300 | 10 | 100 μM PAP (250 μl x10), 100 μM NOS (250 μl x10), 100 μM DIA (250 μl | 30 | 25 |
| | | | x10), 250 μM QUI (250 μl x3), 250 μM CPZ (250 μl x3), 1000 μM PRO (250 μl | | Oxymorphone |
| | | | x3), 1000 μM COC (250 μl x3), 1000 μM LID (250 μl x3), 1000 μM BZC | | |
| | | | (250 μl x3), 1000 M DPH (250 μl x3), 1000 μM LEV (250 μl x3), 1000 μM | | |
| | | | ACM (250 μl x3), 1000 μM NOL (250 μl x10), 1000 μM NAL (250 μl x10), | | |
| | | | 1000 μM mNAL (250 μl x10), 1000 μM CAF (250 μl x3), 1000 μM MAN | | |
| | | | (250 μl x3), 1000 μM LAC (250 μl x3), 1000 μM XYL (250 μl x3), 200 μM | | |
| | | | AF (250 μl x3), 100 μM CLO (250 μl x3), 100 μM ALP (250 μl x3), 1000 μM | | |
| | | | METH (250 μl x3), 1000 μM PSU (250 μl x3), 1000 μM ETY (250 μl x3), | | |
| T4 | 300 | 10 | 100 μM PAP (250 μl x10), 100 μM NOS (250 μl x10), 100 μM DIA (250 μl | 30 | 25 |
| | | | x10), 250 μM QUI (250 μl x3), 250 μM CPZ (250 μl x3), 1000 μM PRO (250 μl | | Oxycodone |
| | | | x3), 1000 μM COC (250 μl x3), 1000 μM LID (250 μl x3), 1000 μM BZC | | |
| | | | (250 μl x3), 1000 μM DPH (250 μl x3), 1000 μM LEV (250 μl x3), 1000 μM | | |
| | | | ACM (250 μl x3), 1000 μM NOL (250 μl x10), 1000 μM NAL (250 μl x10), | | |
| | | | 1000 μM mNAL (250 μl x10), 1000 μM CAF (250 μl x3), 1000 μM MAN | | |
| | | | (250 μl x3), 1000 μM LAC (250 μl x3), 1000 μM XYL (250 μl x3), 200 μM | | |
| | | | AF (250 μl x3), 100 μM CLO (250 μl x3), 100 μM ALP (250 μl x3), 1000 μM | | |
| | | | METH (250 μl x3), 1000 μM PSU (250 μl x3), 1000 μM ETY (250 μl x3), | | |
| T5 | 300 | 10 | 100 μM PAP (250 μl x10), 100 μM NOS (250 μl x10), 100 μM DIA (250 μl | 30 | 10 |
| | | | x10), 250 μM QUI (250 μl x3), 250 μM CPZ (250 μl x3), 1000 μM PRO (250 μl | | Oxymorphone |
| | | | x3), 1000 μM COC (250 μl x3), 1000 μM LID (250 μl x3), 1000 μM BZC | | |
| | | | (250 μl x3), 1000 μM DPH (250 μl x3), 1000 μM LEV (250 μl x3), 1000 μM | | |
| | | | ACM (250 μl x3), 1000 μM NOL (250 μl x10), 1000 μM NAL (250 μl x10), | | |
| | | | 1000 μM mNAL (250 μl x10), 1000 μM CAF (250 μl x3), 1000 μM MAN | | |
| | | | (250 μl x3), 1000 μM LAC (250 μl x3), 1000 μM XYL (250 μl x3), 200 μM | | |
| | | | AF (250 μl x3), 100 μM CLO (250 μl x3), 100 μM ALP (250 μl x3), 1000 μM | | |
| | | | METH (250 μl x3), 1000 μM PSU (250 μl x3), 1000 μM ETY (250 μl x3), | | |
| T6 | 300 | 10 | 100 μM PAP (250 μl x10), 100 μM NOS (250 μl x10), 100 μM DIA (250 μl | 30 | 10 |
| | | | x10), 250 μM QUI (250 μl x3), 250 μM CPZ (250 μl x3), 1000 μM PRO (250 μl | | Oxycodone |
| | | | x3), 1000 μM COC (250 μl x3), 1000 μM LID (250 μl x3), 1000 μM BZC | | |
| | | | (250 μl x3), 1000 μM DPH (250 μl x3), 1000 μM LEV (250 μl x3), 1000 μM | | |
| | | | ACM (250 μl x3), 1000 μM NOL (250 μl x10), 1000 μM NAL (250 μl x10), | | |
| | | | 1000 μM mNAL (250 μl x10), 1000 μM CAF (250 μl x3), 1000 μM MAN | | |
| | | | (250 μl x3), 1000 μM LAC (250 μl x3), 1000 μM XYL (250 μl x3), 200 μM | | |
| | | | AF (250 μl x3), 100 μM CLO (250 μl x3), 100 μM ALP (250 μl x3), 1000 μM | | |
| | | | METH (250 μl x3), 1000 μM PSU (250 μl x3), 1000 μM ETY (250 μl x3), | | |

Counter target abbreviations: Papaverine (PAP), noscapine (NOS), diazepam (DIA), procaine (PRO), quinine (QUI), chlorpromazine (CPZ), cocaine (COC), lidocaine (LID), benzocaine (BZC), diphenhydramine (DPH), levamisole (LEV), acetaminophen (ACM), caffeine (CAF), mannitol (MAN), lactose, (LAC), xylazine (XYL), clonazepam (CLO), alprazolam (ALP), (+)-methamphetamine (METH), (+)-pseudoephedrine (PSU), ethylone (ETY), G1 (combination of PRO, QUI, & CPZ), G2 (combination of COC, LID, & BZC), G3 (combination of DPH, LEV, & ACM), G4 (combination of CAF, MAN, LAC, & XYL), G5 (combination of CLO & ALP), and G6 (combination of METH, PSU, & ETY).

High-throughput sequencing (HTS). Serial selection rounds S1-4 for the morphine and heroin pools and toggle selection rounds T1, T2, T3, T4, and T6 for the oxycodone and oxymorphone pools were subjected to Illumina-based HTS by Azenta Life Sciences. Prior to submission, partial Illumina adapters were added to each sequence via PCR amplification using customized forward and reverse primers (Table 1, FP-HTS and RP-HTS). Specifically, 100 nM of each pool was mixed with 1 μM FP-HTS and RP-HTS and subjected to 10 PCR cycles under the following conditions: 2 min at 95° C.; 9 cycles of 95° C. for 15 s, 58° C. for 30 s, and 72° C. for 45 s; and finally, 5 min at 72° C. The PCR product was confirmed using polyacrylamide gel electrophoresis (PAGE) and then purified using the QIAquick PCR purification kit. A 20 ng/μL solution of the purified pool was submitted for sequencing. For analysis, the constant regions were removed from each pool using cutadapt software. FASTAptamer was then used to obtain the population of each unique sequence during SELEX as well as its enrichment-fold between rounds.

Exonuclease digestion fluorescence assay for aptamer affinity screening. The exonuclease digestion fluorescence assay was performed as previously described. The aptamer (final concentration: 0.5 M) was first diluted in Tris buffer (final concentration: 10 mM, pH 7.4) and heated to 95° C. for 10 min and immediately cooled on ice for 1 min. NaCl (final concentration: 20 mM), MgCl₂ (final concentration: 0.5 mM), and BSA (final concentration: 0.1 mg/mL) were then immediately added. 5 μL of the aptamer solution was then added to 20 μL of buffer, target, or interferent dissolved in selection buffer at various concentrations. The mixture was incubated at 25° C. for 30 min, after which a 25 μL solution of T5 and Exo I (final concentrations: 0.2 U/μL and 0.015 U/μL, respectively) in selection buffer containing 0.1 mg/mL BSA was added to begin the digestion reaction. 5 μL of sample was collected at various time-points and added to 30 μL of quenching solution in the wells of a 384-well black microplate. SYBR Gold fluorescence was recorded using a Tecan Spark plate reader with an excitation wavelength of 495 nm and emission wavelength of 537 nm. The fluorescence was plotted against each time point to construct time-course digestion plots of each sample. Enzymatic inhibition was measured in terms of resistance value which is calculated using the formula (AUCₜ/AUC₀)−1, where AUCₜ and AUC₀ are the areas under the curve of the time-course data with and without target, respectively. The integration time was customized for each aptamer and was chosen as the point at which fluorescence reached 10% of its initial value. The fluorescence of each sample was recorded 10 times, and average values were used for analysis.

Determination of aptamer binding affinities using isothermal titration calorimetry (ITC). ITC experiments were performed in selection buffer at 23° C. using a Malvern MicroCal iTC200 instrument. Aptamer and target concentrations used for ITC experiments are described in Table 7 (for heroin-binding (HM) aptamers) and 8 (for oxycodone-binding (OM) aptamers) below. A 320 μL solution of aptamer in Tris buffer (final concentration: 10 mM, pH 7.4) was heated to 95° C. for 10 min, and then cooled immediately on ice for 1 min. 40 μL of 10×NaCl and 40 μL of 10×MgCl₂ was then added to reach the final selection buffer conditions. 300 μL of the solution was loaded into the cell, and the syringe was loaded with a minimum of 38.4 μL of either morphine or oxycodone dissolved in selection buffer. During ITC experiments, an initial purge injection of 0.4 μL was performed followed by 19 successive injections of 2 μL with a spacing of 180 sec between each injection. The data was then fitted with a one-site binding model using the MicroCal analysis kit integrated into Origin 7 software.

TABLE 7
Aptamer dissociation constants (K_{D}) and ITC conditions for heroin-binding aptamers.

| Aptamer | | [Aptamer] | [Morphine] | ΔH | ΔS | K_{D} |
|---|---|---|---|---|---|---|
| ID | Category | (μM) | (μM) | (kcal/mol) | (cal/mol/K) | (μM) |
| HM1 | 1 | 15 | 200 | −29.4 ± 0.2 | −71.5 | 0.85 ± 0.03 |
| HM2 | 3 | 15 | 200 | −19 ± 0.6 | −39.8 | 4.57 ± 0.25 |
| HM4 | 1 | 15 | 200 | −15.8 ± 0.6 | −27.6 | 2.39 ± 0.25 |
| HM5 | 2 | 15 | 200 | −15.6 ± 0.5 | −27 | 2.61 ± 0.22 |
| HM7 | 3 | 15 | 200 | −17.7 ± 0.9 | −35.4 | 4.74 ± 0.43 |
| HM9 | 1 | 15 | 200 | −21.1 ± 0.4 | −45.6 | 2.4 ± 0.14 |
| HM10 | 1 | 15 | 200 | −19.3 ± 1 | −40.4 | 3.76 ± 0.4 |
| HM11 | 2 | 15 | 200 | −15.7 ± 0.5 | −28.6 | 4.35 ± 0.25 |
| HM14 | 2 | 15 | 200 | −14.8 ± 0.9 | −25.4 | 4.13 ± 0.5 |
| HM15 | 1 | 15 | 200 | −15.9 ± 0.3 | −28.6 | 3.41 ± 0.15 |
| HM16 | 1 | 15 | 200 | −15.1 ± 0.3 | −24.6 | 1.61 ± 0.1 |
| HM20 | 1 | 15 | 200 | −16.5 ± 0.2 | −28.3 | 1.11 ± 0.06 |
| HM22 | 3 | 15 | 200 | −15.1 ± 0.5 | −26.5 | 4.08 ± 0.33 |
| HM23 | 2 | 15 | 200 | −14.2 ± 0.3 | −22.6 | 3.11 ± 0.17 |
| HM24 | 2 | 15 | 200 | −13.9 ± 0.2 | −21.1 | 2.18 ± 0.1 |
| HM25 | 3 | 15 | 200 | −16.7 ± 0.1 | −32.7 | 6.49 ± 0.49 |
| HM29 | 2 | 15 | 200 | −14.7 ± 0.4 | −24 | 2.67 ± 0.17 |
| HM30 | 2 | 15 | 200 | −15.8 ± 0.5 | −27.4 | 2.28 ± 0.2 |
| HM31 | 2 | 15 | 200 | −15.7 ± 0.6 | −28.9 | 5.62 ± 0.44 |
| HM31.2 | 3 | 15 | 200 | −15.1 ± 0.6 | −26 | 3.31 ± 0.28 |
| HM41 | 2 | 15 | 200 | −15.8 ± 0.5 | −28.4 | 3.31 ± 0.28 |
| HM54 | 2 | 15 | 200 | NA | NA | NA |
| HM116 | 1 | 15 | 200 | −14.7 ± 0.2 | −23.5 | 2.11 ± 0.11 |
| HM383 | 1 | 15 | 200 | NA | NA | NA |
| HM426 | 1 | 15 | 200 | −14.8 ± 0.3 | −25.5 | 4.52 ± 0.21 |

TABLE 8
Aptamer dissociation constants (K_{D}) and ITC
conditions for oxycodone-binding aptamers.

| Aptamer | | [Aptamer] | [Oxycodone] | ΔH | ΔS | K_{D} |
|---|---|---|---|---|---|---|
| ID | Category | (μM) | (μM) | (kcal/mol) | (cal/mol/K) | (μM) |
| OM1 | 1 | 15 | 200 | −26.5 ± 0.2 | −60.3 | 0.45 ± 0.03 |
| OM2 | 2 | 15 | 200 | −16.8 ± 0.3 | −29.1 | 0.9 ± 0.07 |
| OM3 | 1 | 15 | 200 | −24.4 ± 0.9 | −57.2 | 3.06 ± 0.29 |
| OM4 | 1 | 15 | 200 | −12.8 ± 0.3 | −15.7 | 0.99 ± 0.13 |
| OM5 | 1 | 15 | 200 | −12.3 ± 0.2 | −12.4 | 0.41 ± 0.05 |
| OM6 | 1 | 20 | 200 | −14.8 ± 0.2 | −21 | 0.5 ± 0.04 |
| OM7 | 1 | 15 | 200 | −22 ± 0.3 | −47.5 | 1.42 ± 0.08 |
| OM8 | 1 | 15 | 200 | −14.8 ± 0.3 | −24.2 | 2.22 ± 0.15 |
| OM9 | 2 | 15 | 200 | −25.3 ± 0.3 | −58.3 | 1.15 ± 0.06 |
| OM10 | 2 | 15 | 200 | −21.9 ± 0.3 | −46.5 | 1.06 ± 0.07 |
| OM11 | 3 | 15 | 200 | −14.8 ± 0.3 | −22.2 | 0.79 ± 0.09 |
| OM12 | 1 | 15 | 200 | −18.9 ± 0.4 | −38.4 | 2.9 ± 0.15 |
| OM13 | 2 | 15 | 200 | −16.5 ± 0.6 | −30.8 | 3.7 ± 0.28 |
| OM14 | 2 | 15 | 200 | −36.1 ± 0.4 | −95.9 | 2.16 ± 0.07 |
| OM15 | 1 | 15 | 200 | −22.5 ± 0.3 | −49.7 | 1.69 ± 0.07 |
| OM16 | 2 | 15 | 200 | −10 ± 0.2 | −6.76 | 1.19 ± 0.13 |
| OM17 | 3 | 15 | 200 | −17.4 ± 0.4 | −33.9 | 3.65 ± 0.21 |
| OM19 | 3 | 15 | 200 | −13.6 ± 0.4 | −20.1 | 2.43 ± 0.18 |
| OM20 | 3 | 15 | 200 | −12.9 ± 0.4 | −17.7 | 2.16 ± 0.19 |
| OM22 | 2 | 15 | 200 | −10.3 ± 0.2 | −8.45 | 1.75 ± 0.12 |
| OM25 | 3 | 15 | 200 | −19.3 ± 0.9 | −41.3 | 6.29 ± 0.42 |
| OM28 | 2 | 15 | 200 | −19 ± 0.9 | −40.3 | 6.13 ± 0.43 |
| OM29 | 1 | 15 | 200 | −25.7 ± 0.2 | −57.9 | 0.51 ± 0.03 |
| OM31 | 2 | 15 | 200 | −17.6 ± 0.6 | −34.4 | 3.19 ± 0.24 |
| OM36 | 2 | 15 | 200 | −18.4 ± 0.3 | −36.6 | 2.77 ± 0.13 |

Optimization of aptamer concentration for the MTC-displacement assay. Each dye-displacement sensor was first tested to identify the optimal aptamer-to-dye ratio as previously described. First, a 99 μL solution of HM20 or OM9 was prepared at various concentrations (final concentrations: 0, 1, 2, 3, 4, 5, 6, 7, 8, 9, and 10 μM) in 1.01× selection buffer containing SDS (final concentration: 0.01% w/v) and incubated at room temperature for 5 mins. 1 μL of 250 μM MTC dissolved in DMSO was then added directly to the solution and rapidly mixed. 72 μL of this mixture was then loaded into the wells of a transparent 384-well microplate, and the absorbance spectra were recorded using a Tecan Spark microplate reader from 400-800 nm with a 5-nm step size.

Detection of opioids using a colorimetric MTC-displacement aptamer assay. The optimal aptamer concentrations for the dye-displacement sensors were 6 and 4 μM for HM20 and OM9, respectively. Each aptamer was diluted to a volume of 39.2 μL at their respective concentrations in 1.01× selection buffer containing SDS (final concentration: 0.01% (w/v)) and incubated at room temperature for 5 mins, after which 0.8 μL of 250 μM MTC was added and rapidly mixed. The mixture was then added to 40 μL of SDS-containing buffer with or without target (heroin for HM20, oxycodone for OM9). Final target concentrations were 0, 0.5, 1, 2, 4, 8, 16, 32, 64, 128, 256, and 512 μM. Finally, 72 μL of the sample was loaded into the wells of a transparent 384-well microplate, and the absorbance spectra were recorded as described above.

For specificity testing, the aptamer-dye complexes were prepared using the same protocol, after which the mixture was added to 40 μL of buffer containing 0.01% SDS, 25 μM target (oxycodone for OM9 and heroin for HM20) in buffer containing 0.01% SDS, or 50 μM interferent (lactose, mannitol, cocaine, benzocaine, naloxone, naltrexone, methylnaltrexone, levamisole, lidocaine, fentanyl, procaine, (+)-pseudoephedrine, diphenhydramine, (+)-methamphetamine, acetaminophen, xylazine, ethylone, alprazolam, diazepam, clonazepam, papaverine, noscapine, quinine, or caffeine) in buffer containing 0.01% SDS. For binary mixture testing, the aptamer-dye complexes were prepared using the same protocol, after which the mixture was added to 40 μL of buffer containing 0.01% SDS, 25 μM target (oxycodone for OM9 and heroin for HM20) dissolved in buffer containing 0.01% SDS, or 25 μM target mixed with 50 μM interferent (lactose, mannitol, cocaine, benzocaine, naloxone, naltrexone, methylnaltrexone, levamisole, lidocaine, fentanyl, procaine, (+)-pseudoephedrine, diphenhydramine, (+)-methamphetamine, acetaminophen, xylazine, ethylone, alprazolam, diazepam, clonazepam, papaverine, noscapine, quinine, or caffeine) in buffer containing 0.01% SDS.

Extraction of pharmaceutical pill contents. Pills tested include Advil (200 mg ibuprofen), Claritin (10 mg loratadine), Benadryl (25 mg diphenhydramine), Mucinex (60 mg dextromethorphan and 1,200 mg guaifenesin), Tylenol (325 mg acetaminophen), Generic hydrocodone (Tris Pharma Inc, G035) (5 mg hydrocodone and 325 mg acetaminophen), and generic oxycodone (4839V) (5 mg oxycodone and 325 mg acetaminophen). Each pill was placed into its own Ziploc bag and gently crushed into a fine powder. The bag contents were then transferred into a 25 mL centrifugation tube after dissolving in 10 mL DI water containing 10% MeOH (v/v) cosolvent and allowed to extract for 30 mins at room temperature on an end-over-end rotator. The extract was then centrifuged at 5,000 ref for 10 mins, and the supernatant was filtered using a 0.45-μm MCE syringe filter.

Detection of opioids in pill extracts using an aptamer-based MTC-displacement assay. OM9 was utilized for oxycodone detection using the optimal aptamer concentration. The aptamer was diluted to a volume of 39.2 μL in 1.01× selection buffer containing SDS (final concentration: 0.01% (w/v)) and allowed to sit at room temperature for 5 mins, after which 0.8 μL of 250 μM MTC was added and rapidly mixed. The mixture was then added to 40 μL of pill extract diluted 5-fold in selection buffer. Finally, 72 μL of the sample was loaded into the wells of a transparent 384-well microplate, and the absorbance spectra were recorded as described above.

Sequences. The various embodiments of the present disclosure include polynucleotides having the following nucleic acid sequences.

The various embodiments of the present disclosure include polynucleotides having the following nucleic acid sequences.

HM1 Family Consensus Sequence (SEQ ID NO: 1): GGATTCG(X)₁₆₋₁₈CTCGT; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; X₁₅ is A, T, C, or G; X₁₆ is A, T, C, or G; X₁₇ is A, T, C, or G; and X₁₈ is A, T, C, or G (SEQ ID NO: 1).

HM1 Family Consensus Sequence (SEQ ID NO: 2): GGATTCG(X)₁₆₋₁₈CTCGT; wherein X₁ is G or C; X₂ is A or G; X₃ is T or C; X₄ is C; X₅ is G or C; X₆ is T or C; X₇ is G or A; X₈ is G or A; X₉ is A or G; X₁₀ is A or T; X₁₁ is C or G; X₁₂ is A or G; X₁₃ is G; X₁₄ is T or G; X₁₅ is G or A; X₁₆ is C or G; X₁₇ is G; and X₁₈ is G (SEQ ID NO: 2).

HM2 Family Consensus Sequence (SEQ ID NO: 3): GCGTA(X)₇TAG(X)₅CGTCGTTCAA; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; and X₁₂ is A, T, C, or G.

HM2 Family Consensus Sequence (SEQ ID NO: 4): GCGTA(X)₇TAG(X)₅CGTCGTTCAA; wherein X₁ is G or C; X₂ is G, C, or T; X₃ is T or A; X₄ is T, C, or G; X₅ is T, C, or G; X₆ is C, G, or A; X₇ is C or G; X₈ is C or T; X₉ is T or C; X₁₀ is G; X₁₁ is T or C; and X₁₂ is G or A.

HM4 Family Consensus Sequence (SEQ ID NO: 5): TAGC(X)₃₋₄GCGTTGTTCGA(X)₆₋₈AGTA; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; and X₁₂ is A, T, C, or G.

HM4 Family Consensus Sequence (SEQ ID NO: 6): TAGC(X)₃₋₄GCGTTGTTCGA(X)₆₋₈AGTA; wherein X₁ is C, G, or T; X₂ is C or G; X₃ is A, T, C, or G; X₄ is G, T, or A; X₅ is G, A, or C; X₆ is T, C, or G; X₇ is A, T, C, or G; X₈ is C, A, or T; X₉ is A, G, or T; X₁₀ is G, T, or C; X₁₁ is C or G; and X₁₂ is G or A.

HM7 Family Consensus Sequence (SEQ ID NO: 7): AGGGCACGTCT(X)₆₋₈AGGGTTTCGCG; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; and X₈ is A, T, C, or G.

HM7 Family Consensus Sequence (SEQ ID NO: 8): AGGGCACGTCT(X)₆₋₈AGGGTTTCGCG; wherein X₁ is C or G; X₂ is G or T; X₃ is G; X₄ is C or T; X₅ is A or G; X₆ is T or C; X₇ is C; and X₈ is G.

OM1 Family Consensus Sequence (SEQ ID NO: 34): (X)₁₂₋₁₃TCTCAGCGAGTTCG(X)₃₋₄; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; X₁₅ is A, T, C, or G; X₁₆ is A, T, C, or G; and X₁₇ is A, T, C, or G.

OM1 Family Consensus Sequence (SEQ ID NO: 35): (X)₁₂₋₁₃TCTCAGCGAGTTCG(X)₃₋₄; wherein X₁ is A, T, or G; X₂ is A, C, or G; X₃ is G, C, or A; X₄ is T or G; X₅ is C or T; X₆ is A; X₇ is G; X₈ is G, C, or T; X₉ is C or T; X₁₀ is C; X₁₁ is T or A; X₁₂ is G or T; X₁₃ is C or G; X₁₄ is A, C, or T; X₁₅ is C, G, or T; X₁₆ is A or G; and X₁₇ is T or A.

OM2 Family Consensus Sequence (SEQ ID NO: 36): GGCT(X)₁₁GTAGGGGT(X)₁CACGCT; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; and X₁₂ is A, T, C, or G.

OM2 Family Consensus Sequence (SEQ ID NO: 37): GGCT(X)₁₁GTAGGGGT(X)₁CACGCT; wherein X₁ is T; X₂ is A or G; X₃ is G or C; X₄ is C or A; X₅ is A or T; X₆ is A or T; X₇ is A or G; X₈ is G or A; X₉ is C or G; X₁₀ is T or C; and X₁₁ is A; and X₁₂ is C or A.

OM4 Family Consensus Sequence (SEQ ID NO: 38): (X)₂₋₆GGGAGT(X)₂₋₃GTTTGTGTGGGG(X)₂₋₆; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; and X₁₅ is A, T, C, or G.

OM4 Family Consensus Sequence (SEQ ID NO: 39): (X)₂₋₆GGGAGT(X)₂₋₃GTTTGTGTGGGG(X)₂₋₆; wherein X₁ is G; X₂ is A or G; X₃ is G; X₄ is T; X₅ is G; X₆ is C; X₇ is G, A, or T; X₈ is T or A; X₉ is T; X₁₀ is T, A, or G; X₁₁ is C or G; X₁₂ is C or T; X₁₃ is C; X₁₄ is G or C; and X₁₅ is A.

OM5 Family Consensus Sequence (SEQ ID NO: 40): (X)₁₋₂CGTAGGGG(X)₁ACACGCT(X)₄₋₆GGGCTG (X)₁₋₂; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; and X₁₀ is A, T, C, or G.

OM5 Family Consensus Sequence (SEQ ID NO: 41): (X)₁₋₂CGTAGGGG(X)₁ACACGCT(X)₄₋₆GGGCTG(X)₁₋₂; wherein X₁ is C or A; X₂ is T or G; X₃ is T, C, or A; X₄ is C or T; X₅ is T, A, or G; X₆ is A or C; X₇ is T, C, or A; X₈ is A or G; X₉ is G; X₁₀ is G or A; and X₁₁ is T or G.

OM12 Family Consensus Sequence (SEQ ID NO: 42): ATGGGAT(X)₁₋₄ATGTGGTGT; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; and X₁₄ is A, T, C, or G.

OM12 Family Consensus Sequence (SEQ ID NO: 43): ATGGGAT(X)₁₋₄ATGTGGTGT; wherein X₁ is A or G; X₂ is C or G; X₃ is G, A, or C; X₄ is A or G; X₅ is A, C, or G; X₆ is C, T, or A; X₇ is T, A, or G; X₈ is C or T; X₉ is G or C; X₁₀ is T or C; X₁₁ is T or G; X₁₂ is T, A, or G; X₁₃ is G; and X₁₄ is G or C.

OM13 Family Consensus Sequence (SEQ ID NO: 44): (X)₃₋₄ATGTGGTGT(X)₇₋₈ATGGGAT(X)₃; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; and X₁₅ is A, T, C, or G.

OM13 Family Consensus Sequence (SEQ ID NO: 45): (X)₃₋₄ATGTGGTGT(X)₇₋₈ATGGGAT(X)₃; wherein X₁ is G; X₂ is C or G; X₃ is G; X₄ is C; X₅ is C; X₆ is G or A; X₇ is G; X₈ is T; X₉ is C; X₁₀ is A; X₁₁ is G or T; X₁₂ is G; X₁₃ is G or C; X₁₄ is A; and X₁₅ is A.

TABLE 9
Aptamer sequences.

| | | SEQ ID |
|---|---|---|
| ID | Sequence (5′-3′) | NO: |
| HM-1 | GGGATTCGGATCGTGGAACAGTGCCTCGTC | 9 |
| HM-54 | GGGATTCGCGCCCCAAGTGGGGAGGGCTCGT | 10 |
| HM-2 | GCGTAGGTTTCCTAGCTGTGCGTCGTTCAA | 11 |
| HM-11 | GCGTAGGTCTCCTAGCTGTGCGTCGTTCAA | 12 |
| HM-15 | GCGTAGGTTCCCTAGCTGTGCGTCGTTCAA | 13 |
| HM-14 | GCGTACCTGTGGTAGTTGTACGTCGTTCAA | 14 |
| HM-22 | GCGTACTTCCAGTAGCCGTGCGTCGTTCAA | 15 |
| HM-31 | GCGTACCATGGGTAGTTGCACGTCGTCCAA | 16 |
| HM-426 | GCGTACCTGTGGTAGTCGTACGTCGTTCAA | 17 |
| HM-4 | TAGCCCGGCGTTGTTCGAGTGCAGCGAGTA | 18 |
| HM-5 | TAGCCGTGGCGTTGTCCGAACCAGTGAGTA | 19 |
| HM-23 | TAGCCGCGCGTTGTCCGACCCTTGGAAGTA | 20 |
| HM-29 | TAGCGCATGCGTTGTTCGAGGATACGAGTA | 21 |
| HM-31.2 | TAGCTGGTGCGTTGTCCGAAGTCATGAGTA | 22 |
| HM-41 | TAGCCCTAGCGTTGTTCGAGTCCATGAGTA | 23 |
| HM-116 | TAGCCGTGGCGTTGTTCGAGTGCAGCGAGTA | 24 |
| HM-24 | AAGTACCAAGGGTAGCTCTGCGTTGTTCGAG | 25 |
| HM-30 | CGTTGTTCGAGCCTTGAGTACCATTGGTAG | 26 |
| HM-7 | AGGGCACGTCTCGGCATCGAGGGTTTCGCG | 27 |
| HM-10 | GAGGGCACGTCTGTGTGCAGGGTTTCGCGC | 28 |
| HM-9 | GGGAAGGTTGGCAGCGCATCGTCGGGTCAC | 29 |
| HM-16 | CGTTGGCGAGTGTGTCCGGGGACTACAGGG | 30 |
| HM-20 | ATGCGTCGAAAAGACCCCTGAGGGCAGCTG | 31 |
| HM-25 | GGGGCTGTGGGAGGGGGTTCATGGGTCCCG | 32 |
| HM-383 | ATGGGATCATGCCTAGTAATGATGTGGTGT | 33 |
| OM-1 | AAGTCAGGCCTGTCTCAGCGAGTTCGACAT | 46 |
| OM-20 | AAGTCAGGTCTGTCTCAGCGAGTTCGACAT | 47 |
| OM-9 | TACGTAGCCCTGTCTCAGCGAGTTCGCGGA | 48 |
| OM-10 | AGAGTAGGCCTGTCTCAGCGAGTTCGCTAT | 49 |
| OM-8 | GCAGCAGTCCATCTCTCAGCGAGTTCGTCA | 50 |
| OM-29 | TGAGTAGGCCTGTCTCAGCGAGTTCACTAT | 51 |
| OM-2 | GGCTTAGCAAAGCTAGTAGGGGTCCACGCT | 52 |
| OM-11 | GGCTTGCATTGAGCAGAAGGGGTACACGCT | 53 |
| OM-4 | GAGTGCGGGGAGTGTGTTTCTGTGGGGGTC | 54 |
| OM-16 | GGGGGGAGTAATGTTTGTGTGGGGGAGCCG | 55 |
| OM-22 | GGGGGGAGTTTGTTTGTGTGGGAGGGTCCA | 56 |
| OM-5 | CCGTAGGGGTACACGCTCTATAGGGGCTGG | 57 |
| OM-6 | CCGTAGGGGTACACGCTCTATGGGGGCTGG | 58 |
| OM-7 | ATCGTAGGGGCACACGCTTAACGGGCTGAT | 59 |
| OM-19 | CGCGTAGGGGAACACGCTTGCAGGGCTGAG | 60 |
| OM-12 | ATGGGATACGAACTCGTTTGGATGTGGTGT | 61 |
| OM-31 | ATGGGATGGAGCTATGCGAGCATGTGGTGT | 62 |
| OM-36 | ATGGGATACCAGAGTCCTGGGATGTGGTGT | 63 |
| OM-13 | GCGCATGTGGTGTCGGTCAGATGGGATGAA | 64 |
| OM-17 | GGGATGTGGTGTCAGTCATGATGGGATCAA | 65 |
| OM-3 | TGGCGCCGGATTCCCTAAGGTACACGGACT | 66 |
| OM-14 | GGATTGCGCTTGCTAACGCCGTGTAGAGTC | 67 |
| OM-15 | CGGACTTGGGTGGGTGCTGGGGCTTGGGCG | 68 |
| OM-25 | GGCCATGGGATTGTCTAATGTGGTGTGGAA | 69 |
| OM-28 | GCGTGGGATACCTTTTGGGATGTGGTGCAC | 70 |

### 5. EXAMPLES

The accompanying Examples are offered as illustrative as a partial scope and particular embodiments of the disclosure and are not meant to be limiting of the scope of the disclosure.

The present disclosure describes the development of several sets of opioid-binding aptamers with distinct specificity profiles and the organic dye-displacement assays for detecting opioids in samples rapidly and with high specificity. As described herein, the ability to rapidly identify opioid compounds in seized drug samples on-site would be a key tool for curtailing trafficking and distribution. Although chemical reagent-based tests are fast and simple it has repeatedly been shown that these tests are prone to giving false results due to poor specificity. Reagent-based tests such as the portable Raman spectrometers, are considered the current alternative to chemical reagent-based tests as they have been shown to have excellent selectivity to opioids however, these spectrometers have often faced interference challenges with impure drug samples decreasing their capability. In this work, on-site sensors for morphine and structurally related opioid compounds based on in vitro-selected oligonucleotide affinity reagents are known as aptamers. The approach employed is a parallel-and-serial selection strategy to isolate aptamers that recognize heroin, morphine, codeine, hydrocodone, and hydromorphone, along with a toggle-selection approach to isolate aptamers that bind oxycodone and oxymorphone. Next, a novel high-throughput sequencing-based approach was utilized to examine aptamer growth patterns over the course of selection, and a high-throughput exonuclease-based screening assay to identify optimal aptamer candidates. Lastly, two high-performance aptamers were used to develop colorimetric dye-displacement assays that can specifically detect opioids in complex chemical matrices, including pharmaceutical tablets and drug mixtures. These aptamer-based colorimetric assays enable naked-eye identification of specific opioids within seconds and will potentially play an important role in combatting opioid abuse.

### Example 1

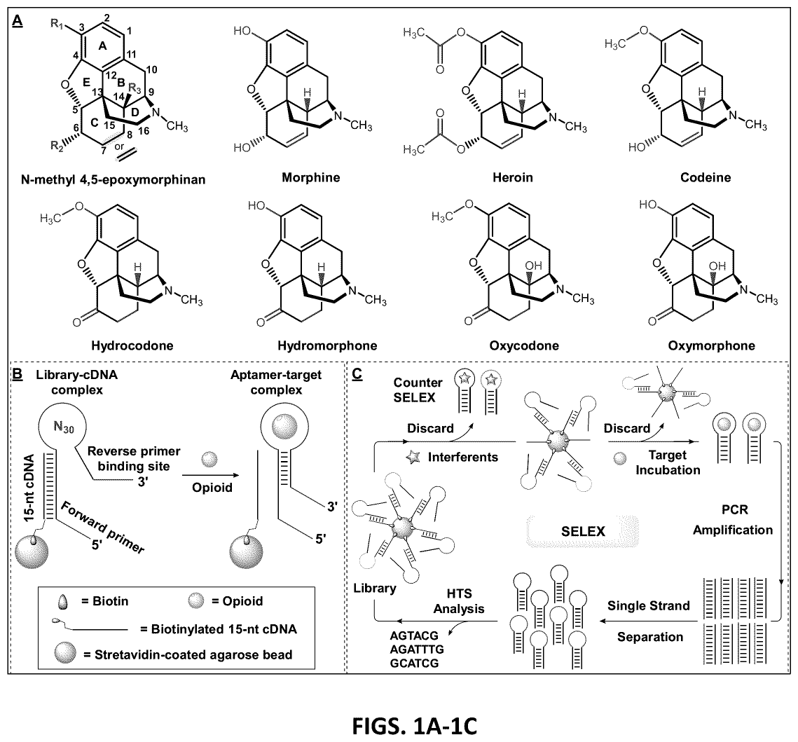

Choice of selection targets and selection strategy. Opioids with the structural similarity to morphine include the opiate codeine and the semi-synthetic opioids heroin, hydrocodone, hydromorphone, oxycodone, and oxymorphone. Morphine, codeine, heroin, hydrocodone, and hydromorphone share the same N-methyl 4,5-epoxymorphinan core structure, while oxycodone and oxymorphone contain a similar, yet distinct, core that contains a hydroxyl group at carbon 14 (C14) in place of hydrogen (FIG. 1A). Categorized are targets into two different groups based on these differences in their core structure. The goal was to isolate two different aptamers that could recognize these different core structures while tolerating alterations at other substituent sites. Therefore, performed were two independent selections using representative molecules from each subfamily: one using morphine and heroin as targets, and the other using oxycodone and oxymorphone as targets. Next, performed was a library-immobilized SELEX with a 73-nucleotide (nt) stem-loop-structured DNA library as previously reported. Each library molecule contains a 30-nt random domain serving as the putative binding domain, flanked by an 8-base-pair (bp) stem with two primer-binding sites at both termini for PCR amplification. The library can hybridize to a 15-nt biotinylated complementary DNA (cDNA15-bio), which facilitates immobilization of the library-cDNA complex onto streptavidin-modified agarose beads. Upon binding of the selection target to a library strand, the aptamer undergoes a conformational change, becomes displaced from the cDNA, and is released into solution (FIG. 1B). These target binders are collected, and PCR amplified to generate a new pool for another round of selection until the pool displays high affinity for the target(s) (FIG. 1C).

### Example 2

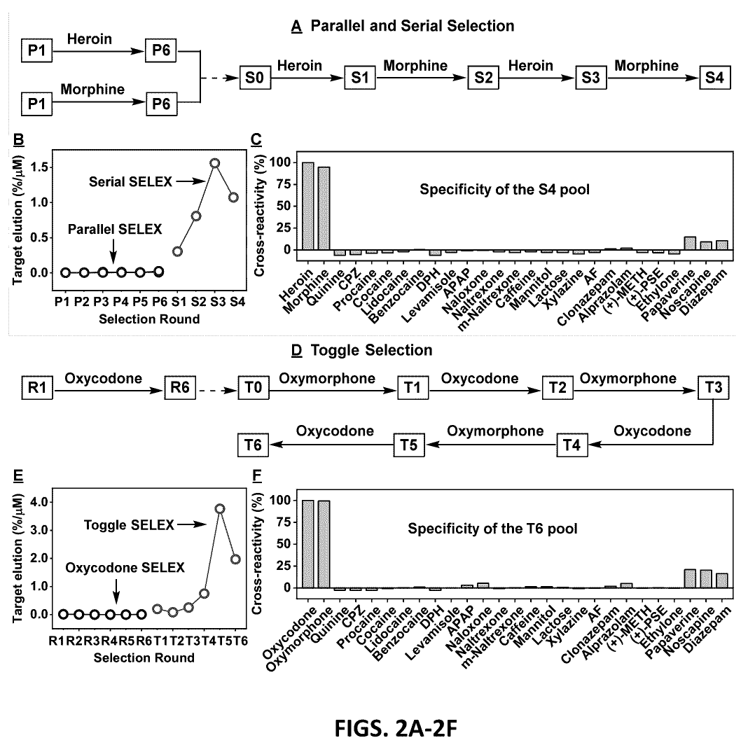

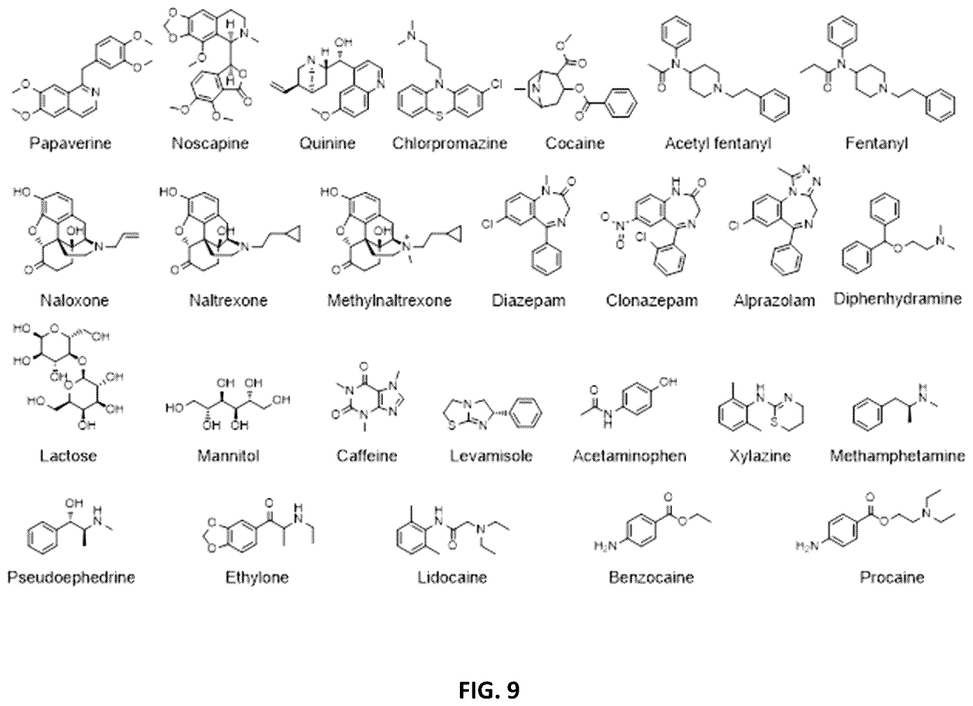

Isolation of cross-reactive aptamers via parallel-and-serial selection using morphine and heroin and toggle selection for oxycodone and oxymorphone aptamers. To enrich aptamers that can broadly recognize morphine-related opioids, the parallel-and-serial selection strategy was utilized to isolate class-specific aptamers for the synthetic cathinone drug family. Initially performed were two independent selections using morphine and heroin as targets in parallel for six rounds to enrich target-binding sequences (FIG. 2A, Rounds P1-P6). For Rounds P1-P5, 500 μM target were employed, reducing target concentration to 250 μM in Round P6. Beginning from Round P2, counter-SELEX was employed to remove binders to interferents such as cutting agents (caffeine, lactose, and mannitol), adulterants (quinine, chlorpromazine, procaine, lidocaine, benzocaine, diphenhydramine, levamisole, acetaminophen, and xylazine), controlled substances (cocaine, diazepam, alprazolam, clonazepam, acetyl fentanyl, (+)-methamphetamine, (+)-pseudoephedrine, and ethylone), endogenous compounds in the opium plant (papaverine and noscapine), and opioid receptor antagonists (naloxone, naltrexone, and methylnaltrexone) (see FIG. 9 for chemical structures). Counter-targets were used at a concentration of 200-500 μM and were employed individually or as a group; detailed selection conditions are provided in Tables 2 and 3.

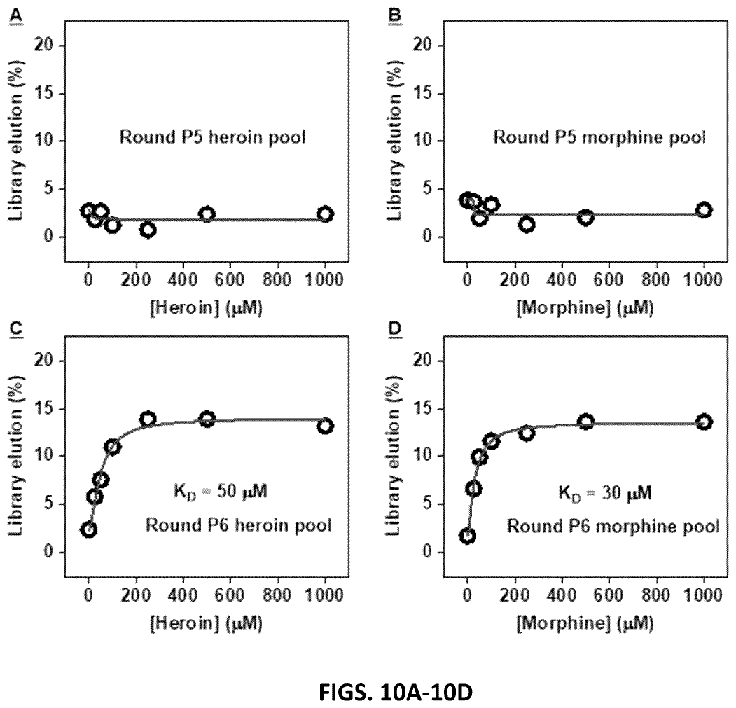

During Rounds P1-P6, the proportion of pool eluted by the targets (0.3-2%), even when adjusted as per micromolar target concentration, was relatively low (FIG. 2B). However, certain counter-targets such as papaverine and noscapine eluted a sizeable quantity of pool during these rounds, ranging from 8-23%. This can be attributed to the fact that these substances are capable of non-specifically binding double-stranded DNA. Also, observed was that counter-target group 1 (procaine, quinine, and chlorpromazine) and group 2 (cocaine, lidocaine, and benzocaine) demonstrated high elution relative to the targets, ranging from 4-6%. Surprisingly, the most structurally-similar counter-targets, naloxone, naltrexone, and methylnaltrexone, eluted just 1-2% of the library. After a gel elution assay was employed to determine the target-binding affinity of the Rounds P5 and P6 heroin and morphine pools. For both P5 pools, no target binding was observed, indicating that these pools had not been sufficiently enriched with target binders (FIG. 10A, B). However, the Round P6 heroin and morphine pools displayed dissociation constants (K_{D}) of 50 μM (FIG. 10C) and 30 μM (FIG. 10D), respectively, with maximum elution of ˜14% for their respective targets. These results indicated that both pools had been enriched with target-binding sequences.

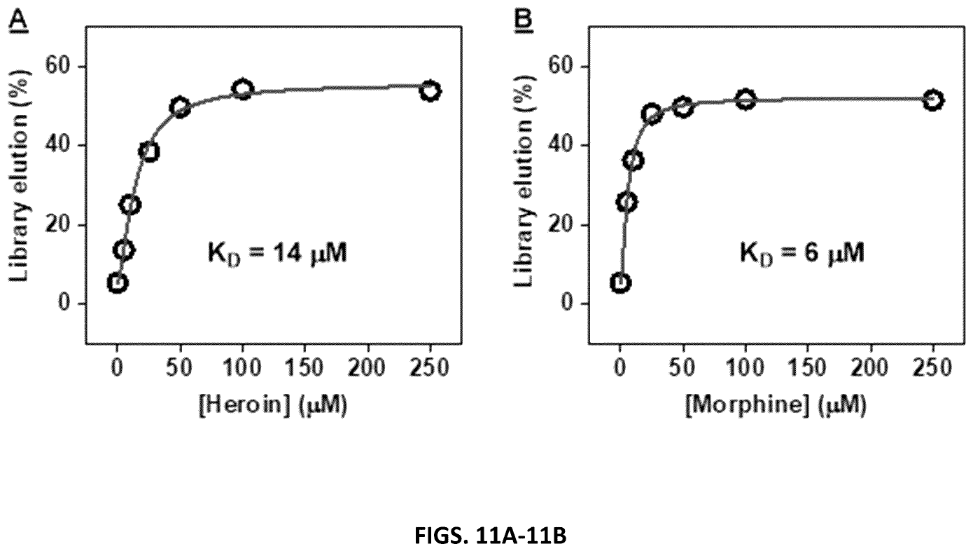

Next, an equimolar quantity of the heroin and morphine Round P6 pools were combined and performed two cycles of serial selection (FIG. 2A, Rounds S1-S4). Each cycle consisted of first challenging the pool with heroin, PCR amplifying binders, and then challenging the resultant pool with morphine. The rationale underlying this strategy is that aptamers that can recognize both targets—and perhaps target analogs like codeine, hydrocodone, and hydromorphone—will be preferentially enriched relative to those that bind only one target. In Rounds S1 and S2, 50 μM heroin and morphine was used, while 25 μM was used for each target in Rounds S3 and S4. To increase the stringency of counter-SELEX, all interferents were used individually rather than as a group, and their concentrations were increased to 1 mM when possible; since papaverine, noscapine, diazepam, alprazolam, clonazepam, quinine, and chlorpromazine have poor solubility in the selection buffer, they were used at lower concentrations (Table 4). For Rounds S1 and S2, heroin eluted 4.5% of the pool, while morphine eluted 12.1%. In Rounds S3 and S4, pool elution rose to 11.7% for heroin and 16.1% for morphine. The proportion of pool eluted per micromolar concentration of target applied is shown in FIG. 2B. Next, the binding affinity and specificity of the resulting S4 pool using the gel-elution assay were characterized. The pool demonstrated a K_{D} of 14 μM and 6 μM for heroin and morphine (FIG. 11), respectively, and had minimal response to most counter-targets at a ten-fold higher concentration relative to the targets, including naloxone, naltrexone, and methylnaltrexone (papaverine, noscapine, diazepam, alprazolam, and clonazepam were used at four-fold higher concentration) (FIG. 2C). However, papaverine, noscapine, and diazepam demonstrated cross-reactivities of 14.8, 9.2, and 10.6%, respectively. Nevertheless, since the pool bound to the two selection targets with decent affinity, which concluded this selection.

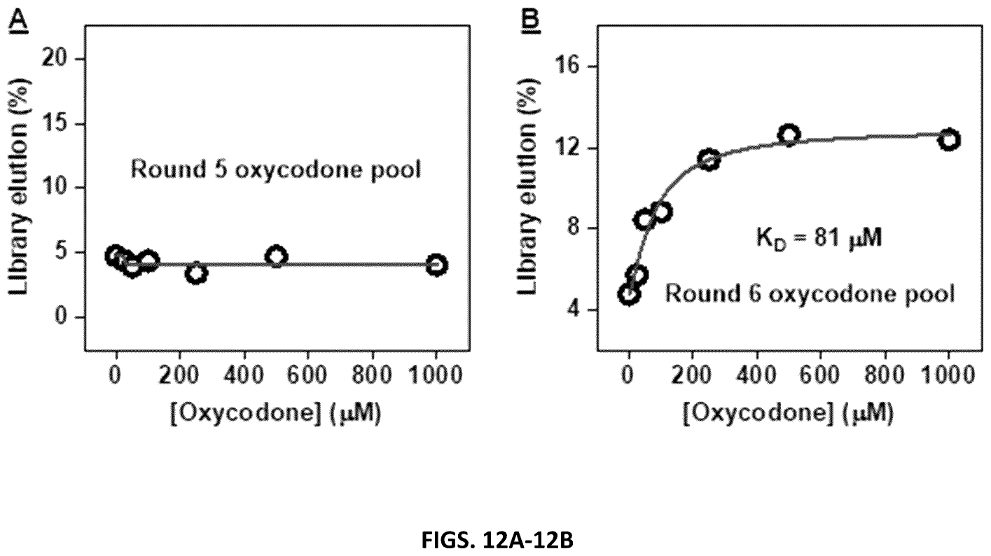

A separate SELEX experiment was preformed using oxycodone and oxymorphone as the selection targets (FIG. 2D, Rounds R1-R6). For this experiment, 500 μM of oxycodone for Round R1-5 was used, reducing this to 250 μM for R6. Oxycodone eluted a relatively low proportion of library during these rounds (0.4-1.2%; percentage of pool eluted per micromolar of target is shown in FIG. 2E). Starting from Round R2, counter-SELEX similar to the morphine-heroin selection were preformed (Table 5). Papaverine, noscapine, diazepam, and a combination of cocaine, lidocaine, and benzocaine eluted ˜20% of the pool in Rounds R2-4. In Rounds R4-6, chlorpromazine, acetyl fentanyl, and naloxone also demonstrated increased pool elution relative to earlier rounds. The binding affinity of the Rounds R5 and R6 pools was determined using a gel elution assay. Although the R5 pool did not have any affinity for oxycodone (FIG. 12A), the R6 pool had a K_{D} of 81 μM and maximum pool elution of 12% (FIG. 12B).

To enrich aptamers that cross-react to oxycodone and oxymorphone, toggle SELEX strategy was applied as previously reported, wherein first applied was oxymorphone as a target and then oxycodone the next round, alternating targets every round for a total of six rounds (FIG. 2D, Rounds T1-T6). Detailed selection conditions are shown in Table 6. For Rounds T1 and T2, 100 μM target was used, achieving pool elution of 6% and 10%, respectively. However, when target concentrations were reduced to 25 μM in Rounds T3 and T4, pool elution respectively decreased to 3.8% and 5.6%. Nevertheless, the pools from these rounds eluted more when adjusted per micromolar concentration of target relative to Rounds T1-2 (FIG. 2E). When target concentrations were reduced to 10 μM in Rounds T5 and T6, absolute pool elution rose again to 11.3% and 11.8%, respectively, indicating that cross-reactive aptamers were successfully being enriched.

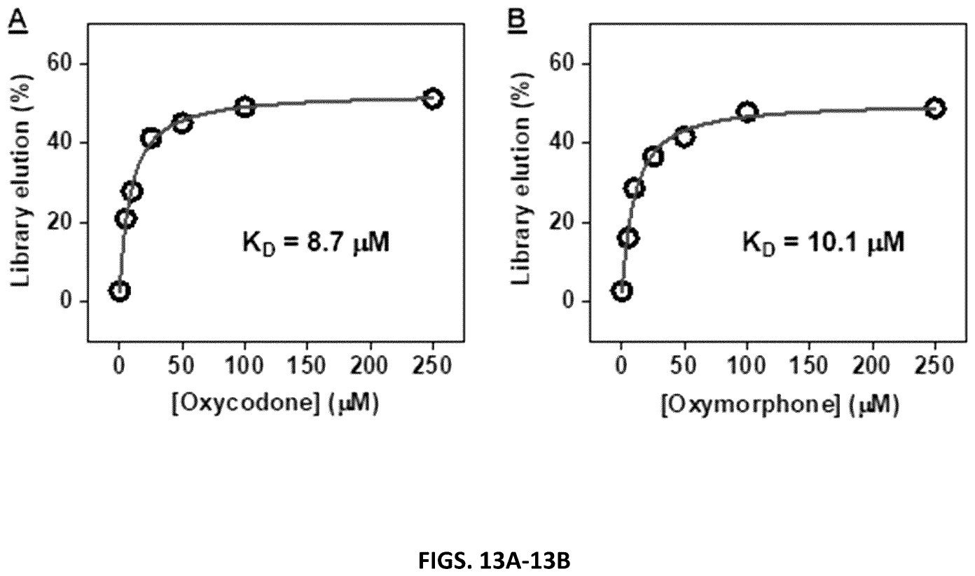

Throughout the toggle rounds, it was noted that papaverine, noscapine, and diazepam consistently eluted a considerable amount of library. More importantly, the structurally similar counter-targets naloxone and naltrexone were responsible for the highest levels of pool elution, with an average of 5% for naloxone. After Round T6, the gel elution assay to characterize the pool's binding properties and determined K_{D}s of 8.7 and 10.1 μM for oxycodone and oxymorphone was performed, respectively (FIG. 13). This pool also demonstrated high specificity, with no response observed against 10-fold higher concentrations of most interferents, even naloxone, although 4-fold higher concentrations of papaverine, noscapine, and diazepam produced cross-reactivities of 20.9, 20.2, and 16.3%, respectively (FIG. 2F). Therefore, the toggle selection was successful in isolating cross-reactive aptamers for oxycodone and oxymorphone.

### Example 3

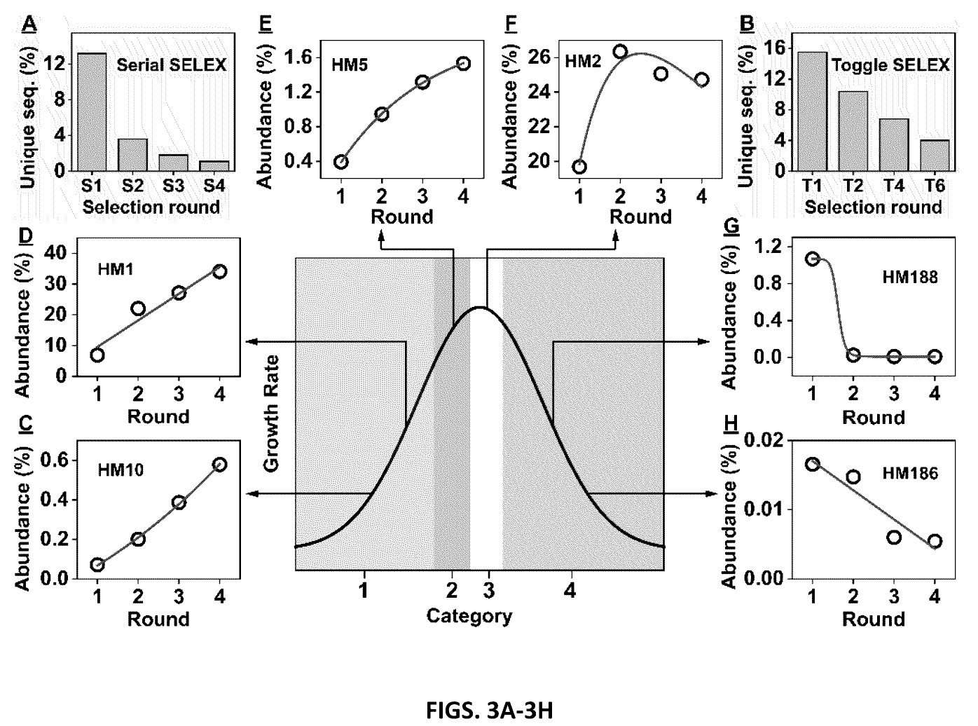

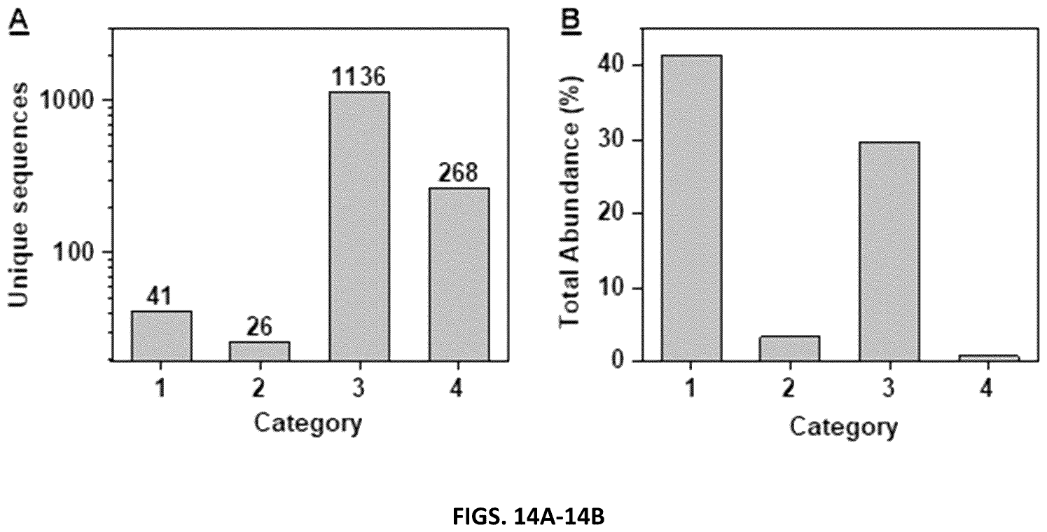

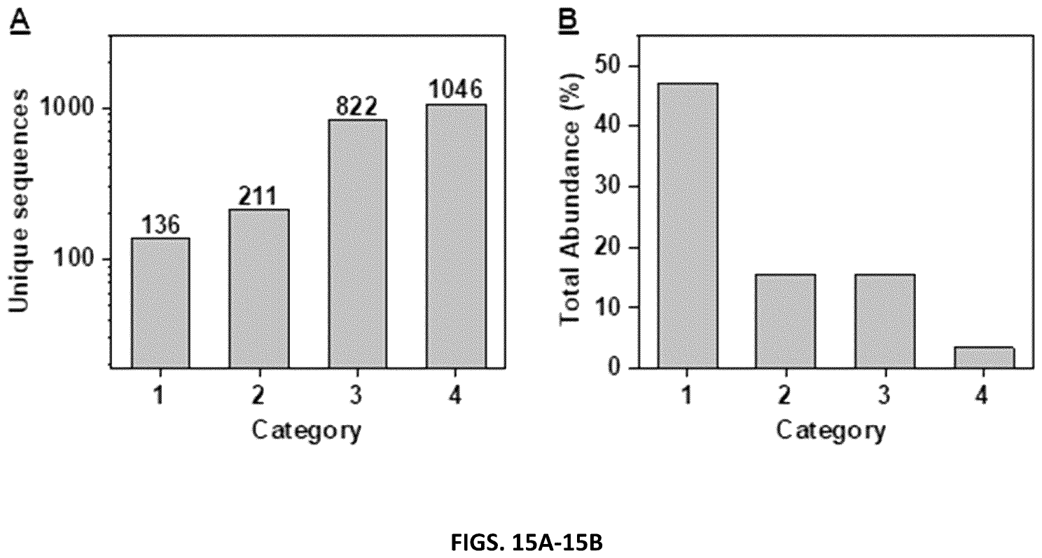

HTS analysis and affinity measurement with ITC. To identify aptamer candidates, four pools from the heroin-morphine (HM) SELEX and five pools from the oxycodone-oxymorphone (OM) SELEX for HTS were submitted. From the HM pools, pools S1-4, obtaining 198,943, 189,690, 231,111, and 257,228 reads, were sequenced respectively. For the OM pools, T1-4 and T6, obtaining 189,685, 219,578, 184,110, 185,984, and 159,168 reads, were sequenced respectively. The diversity of sequences in the pool decreased considerably during selection, with the percentage of unique sequences of the serial selection HM pools decreasing from 13.2 in S1 to 1.1% in the final round and for toggle-selection OM pools decreasing from 15.5 in T1 to 4.0% in T6 (FIG. 3A, B). Overall, the OM pool was less enriched at every round relative to the HM pool. Aptamer candidates were chosen from each pool based on their enrichment behavior. Specifically, candidates were separated into four categories based on evolutionary trends (Tables 10-11, below), in which sequence abundance: 1) continuously increased with an exponential or linear growth rate (FIG. 3C, D), 2) consistently increased with a gradually diminishing growth rate (FIG. 3E), 3) peaked at some point (FIG. 3F), or 4) always decreased between rounds (FIG. 3G, H). It was hypothesized that aptamers from category 1 most likely had the highest target-binding affinities, whereas those from categories 2 and 3 would possess moderate binding affinity. For the final heroin and morphine S4 pool, 41% of the pool (41 unique sequences) was classified as category 1, 3.4% (26 unique sequences) was in category 2, 30% (1136 unique sequences) was in category 3, 0.8% (268 unique sequences) was in category 4, and 25% did not meet criteria for any category (FIG. 14A, B). For the final oxycodone and oxymorphone T6 pool, 47% of the pool (136 unique sequences) was classified as category 1, 15.5% (211 unique sequences) was in category 2, 15.5% (822 unique sequences) was in category 3, 3.5% (1046 unique sequences) was in category 4, and ˜18% did not meet criteria for any category (FIG. 15).

TABLE 10
DNA sequences from parallel-and-serial selection using morphine and
heroin.

| Seq. ID | Category | Sequence (5′ to 3′) |
|---|---|---|
| HM1 | 1 | GGGACGACGGGATTCGGATCGTGGAACAGTGCCTCGTCGTCGTCCC |
| | | (SEQ ID NO: 77) |
| HM2 | 3 | GGGACGACGCGTAGGTTTCCTAGCTGTGCGTCGTTCAAGTCGTCCC (SEQ |
| | | ID NO: 78) |
| HM4 | 1 | GGGACGACTAGCCCGGCGTTGTTCGAGTGCAGCGAGTAGTCGTCCC |
| | | (SEQ ID NO: 79) |
| HM5 | 2 | GGGACGACTAGCCGTGGCGTTGTCCGAACCAGTGAGTAGTCGTCCC |
| | | (SEQ ID NO: 80) |
| HM7 | 3 | GGGACGACAGGGCACGTCTCGGCATCGAGGGTTTCGCGGTCGTCCC |
| | | (SEQ ID NO: 81) |
| HM9 | 1 | GGGACGACGGGAAGGTTGGCAGCGCATCGTCGGGTCACGTCGTCCC |
| | | (SEQ ID NO: 82) |
| HM10 | 1 | GGGACGACGAGGGCACGTCTGTGTGCAGGGTTTCGCGCGTCGTCCC |
| | | (SEQ ID NO: 83) |
| HM11 | 2 | GGGACGACGCGTAGGTCTCCTAGCTGTGCGTCGTTCAAGTCGTCCC (SEQ |
| | | ID NO: 84) |
| HM14 | 2 | GGGACGACGCGTACCTGTGGTAGTTGTACGTCGTTCAAGTCGTCCC (SEQ |
| | | ID NO: 85) |
| HM15 | 1 | GGGACGACGCGTAGGTTCCCTAGCTGTGCGTCGTTCAAGTCGTCCC (SEQ |
| | | ID NO: 86) |
| HM16 | 1 | GGGACGACCGTTGGCGAGTGTGTCCGGGGACTACAGGGGTCGTCCC |
| | | (SEQ ID NO: 87) |
| HM20 | 1 | GGGACGACATGCGTCGAAAAGACCCCTGAGGGCAGCTGGTCGTCCC |
| | | (SEQ ID NO: 88) |
| HM22 | 3 | GGGACGACGCGTACTTCCAGTAGCCGTGCGTCGTTCAAGTCGTCCC |
| | | (SEQ ID NO: 89) |
| HM23 | 2 | GGGACGACTAGCCGCGCGTTGTCCGACCCTTGGAAGTAGTCGTCCC |
| | | (SEQ ID NO: 90) |
| HM24 | 2 | GGGACGACAAGTACCAAGGGTAGCTCTGCGTTGTTCGAGGTCGTCCC |
| | | (SEQ ID NO: 91) |
| HM25 | 3 | GGGACGACGGGGCTGTGGGAGGGGGTTCATGGGTCCCGGTCGTCCC |
| | | (SEQ ID NO: 92) |
| HM29 | 2 | GGGACGACTAGCGCATGCGTTGTTCGAGGATACGAGTAGTCGTCCC |
| | | (SEQ ID NO: 93) |
| HM30 | 2 | GGGACGACCGTTGTTCGAGCCTTGAGTACCATTGGTAGGTCGTCCC (SEQ |
| | | ID NO: 94) |
| HM31 | 2 | GGGACGACGCGTACCATGGGTAGTTGCACGTCGTCCAAGTCGTCCC |
| | | (SEQ ID NO: 95) |
| HM31.2 | 3 | GGGACGACTAGCTGGTGCGTTGTCCGAAGTCATGAGTAGTCGTCCC |
| | | (SEQ ID NO: 96) |
| HM41 | 2 | GGGACGACTAGCCCTAGCGTTGTTCGAGTCCATGAGTAGTCGTCCC |
| | | (SEQ ID NO: 97) |
| HM54 | 2 | GGGACGACGGGATTCGCGCCCCAAGTGGGGAGGGCTCGTGTCGTCCC |
| | | (SEQ ID NO: 98) |
| HM116 | 1 | GGGACGACTAGCCGTGGCGTTGTTCGAGTGCAGCGAGTAGTCGTCCC |
| | | (SEQ ID NO: 99) |
| HM383 | 1 | GGGACGACATGGGATCATGCCTAGTAATGATGTGGTGTGTCGTCCC |
| | | (SEQ ID NO: 100) |
| HM426 | 1 | GGGACGACGCGTACCTGTGGTAGTCGTACGTCGTTCAAGTCGTCCC |
| | | (SEQ ID NO: 101) |

TABLE 11
DNA sequences from toggle selection using oxycodone and oxymorphone.

| Seq. ID | Category | Sequence (5′ to 3′) |
|---|---|---|
| OM1 | 1 | GGGACGACAAGTCAGGCCTGTCTCAGCGAGTTCGACATGTCGTCCC |
| | | (SEQ ID NO: 102) |
| OM2 | 2 | GGGACGACGGCTTAGCAAAGCTAGTAGGGGTCCACGCTGTCGTCCC |
| | | (SEQ ID NO: 103) |
| OM3 | 1 | GGGACGACTGGCGCCGGATTCCCTAAGGTACACGGACTGTCGTCCC |
| | | (SEQ ID NO: 104) |
| OM4 | 1 | GGGACGACGAGTGCGGGGAGTGTGTTTCTGTGGGGGTCGTCGTCCC |
| | | (SEQ ID NO: 105) |
| OM5 | 1 | GGGACGACCCGTAGGGGTACACGCTCTATAGGGGCTGGGTCGTCCC |
| | | (SEQ ID NO: 106) |
| OM6 | 1 | GGGACGACCCGTAGGGGTACACGCTCTATGGGGGCTGGGTCGTCCC |
| | | (SEQ ID NO: 107) |
| OM7 | 1 | GGGACGACATCGTAGGGGCACACGCTTAACGGGCTGATGTCGTCCC |
| | | (SEQ ID NO: 108) |
| OM8 | 1 | GGGACGACGCAGCAGTCCATCTCTCAGCGAGTTCGTCAGTCGTCCC |
| | | (SEQ ID NO: 109) |
| OM9 | 2 | GGGACGACTACGTAGCCCTGTCTCAGCGAGTTCGCGGAGTCGTCCC |
| | | (SEQ ID NO: 110) |
| OM10 | 2 | GGGACGACAGAGTAGGCCTGTCTCAGCGAGTTCGCTATGTCGTCCC |
| | | (SEQ ID NO: 111) |
| OM11 | 3 | GGGACGACGGCTTGCATTGAGCAGAAGGGGTACACGCTGTCGTCCC |
| | | (SEQ ID NO: 112) |
| OM12 | 1 | GGGACGACATGGGATACGAACTCGTTTGGATGTGGTGTGTCGTCCC |
| | | (SEQ ID NO: 113) |
| OM13 | 2 | GGGACGACGCGCATGTGGTGTCGGTCAGATGGGATGAAGTCGTCCC |
| | | (SEQ ID NO: 114) |
| OM14 | 2 | GGGACGACGGATTGCGCTTGCTAACGCCGTGTAGAGTCGTCGTCCC |
| | | (SEQ ID NO: 115) |
| OM15 | 1 | GGGACGACCGGACTTGGGTGGGTGCTGGGGCTTGGGCGGTCGTCCC |
| | | (SEQ ID NO: 116) |
| OM16 | 2 | GGGACGACGGGGGGAGTAATGTTTGTGTGGGGGAGCCGGTCGTCCC |
| | | (SEQ ID NO: 117) |
| OM17 | 3 | GGGACGACGGGATGTGGTGTCAGTCATGATGGGATCAAGTCGTCCC |
| | | (SEQ ID NO: 118) |
| OM19 | 3 | GGGACGACCGCGTAGGGGAACACGCTTGCAGGGCTGAGGTCGTCCC |
| | | (SEQ ID NO: 119) |
| OM20 | 3 | GGGACGACAAGTCAGGTCTGTCTCAGCGAGTTCGACATGTCGTCCC |
| | | (SEQ ID NO: 120) |
| OM22 | 2 | GGGACGACGGGGGGAGTTTGTTTGTGTGGGAGGGTCCAGTCGTCCC |
| | | (SEQ ID NO: 121) |
| OM25 | 3 | GGGACGACGGCCATGGGATTGTCTAATGTGGTGTGGAAGTCGTCCC |
| | | (SEQ ID NO: 122) |
| OM28 | 2 | GGGACGACGCGTGGGATACCTTTTGGGATGTGGTGCACGTCGTCCC |
| | | (SEQ ID NO: 123) |
| OM29 | 1 | GGGACGACTGAGTAGGCCTGTCTCAGCGAGTTCACTATGTCGTCCC |
| | | (SEQ ID NO: 124) |
| OM31 | 2 | GGGACGACATGGGATGGAGCTATGCGAGCATGTGGTGTGTCGTCCC |
| | | (SEQ ID NO: 125) |
| OM36 | 2 | GGGACGACATGGGATACCAGAGTCCTGGGATGTGGTGTGTCGTCCC |
| | | (SEQ ID NO: 126) |

### Example 4

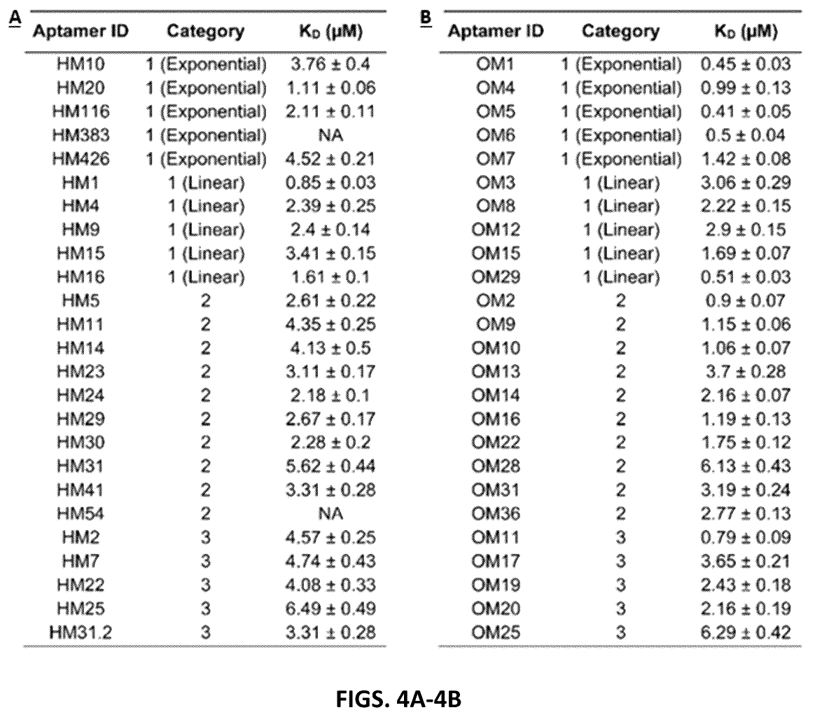

HM and OM pools affinity characterization. The top 10 candidates from category 1 (5 linear and 5 exponential), top 10 candidates from category 2, and top five candidates from category 3 from the HM and OM pools for further affinity characterization. The gold-standard method ITC was used to determine the K_{D} of the 50 candidates against morphine for the HM aptamers or oxycodone for the OM aptamers. K_{D} values ranged from 0.84-6.49 μM and 0.41-6.30 μM for HM and OM aptamers, respectively (FIG. 4A, B). On average, aptamers from category 1 had higher affinity than those in category 2 and 3. For example, the K_{D} of HM aptamers from category 1 ranged from 0.84-4.52 μM, while those from category 2 ranged from 2.18-5.61 μM and category 3 ranged from 3.31-6.49 μM. A similar pattern held for the OM aptamers. It was found that while HM383 and HM54 were promising category 1 and 2 sequences, respectively, ITC indicated they did not bind the target.

### Example 5

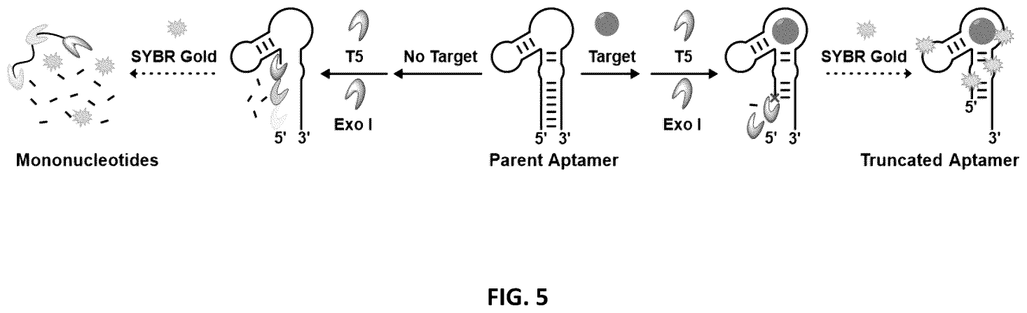

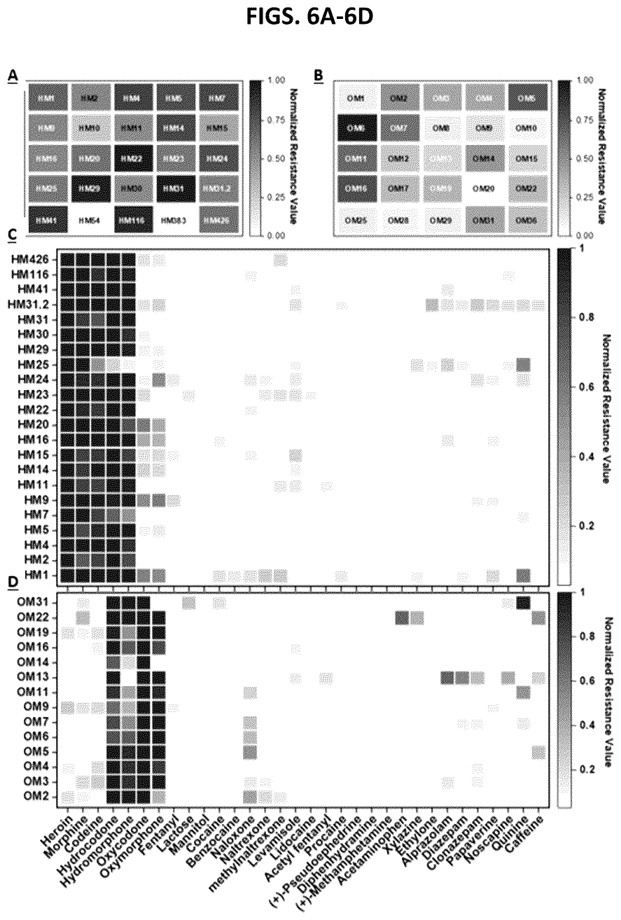

Binding characterization of aptamer candidates using an exonuclease-based fluorescence assay. The binding properties of each aptamer candidate was characterized using a fluorescence assay based on the 5′-3′ DNA exonuclease, T5 Exonuclease (T5 Exo), and the 3′-5′ single-stranded DNA exonuclease, Exonuclease I (Exo I). T5 Exo and Exo I digest DNA aptamers into mononucleotides in the absence of ligand, but digestion is inhibited when the aptamer is bound to a ligand in a manner dependent on ligand concentration and aptamer-ligand affinity (FIG. 5). Monitoring the digestion process over time (for instance, with a DNA-binding fluorescent stain like SYBR Gold) provided a measure of ligand binding affinity and specificity. This interaction was characterized using the metric resistance value (Rᵥₐₗᵤₑ), which is equal to the ratio of the area under the curve (AUC) of the digestion time-course plot with versus without target minus 1. To begin, each candidate was digested with T5 Exo and Exo I in the absence or presence of 100 μM heroin for HM aptamers and 100 μM oxycodone for OM aptamers. In total, 22 HM candidates (HM1, HM2, HM4, HM5, HM7, HM9, HM11, HM14, HM15, HM16, HM20, HM22, HM23, HM24, HM25, HM29, HM30, HM31, HM31.2, HM41, HM116, and HM426) (FIG. 6A) and 14 OM candidates (OM2, OM3, OM4, OM5, OM6, OM7, OM9, OM11, OM13, OM14, OM16, OM19, OM22, and OM31) demonstrated moderate to high enzymatic inhibition (Rᵥₐₗᵤₑ>0.2) (FIG. 6B). All other aptamers had little to no inhibition (Rᵥₐₗᵤₑ<0.2). Next, assessed was the cross-reactivity of the 22 HM aptamers and 14 OM aptamers to seven opioid analogs (heroin, morphine, codeine, oxycodone, hydrocodone, oxymorphone, and hydromorphone) at a concentration of 100 μM using the T5/Exo I assay. Except for HM25, most HM aptamers demonstrated the ability to bind heroin, morphine, codeine, hydrocodone, and hydromorphone with similar levels of enzymatic inhibition. Only five aptamers (HM1 HM9, HM16, HM20, and HM24) also responded to oxycodone and oxymorphone with a cross-reactivity of 40-60% (FIG. 6C). All OM aptamers were able to bind oxycodone and oxymorphone with high cross-reactivity (80-112%), but these also had differing cross-reactivity for the other opioids. In particular, OM3, OM4, and OM5 also cross-reacted to hydrocodone and hydromorphone at a level of 80-130%. However, none of these aptamers could recognize morphine, heroin, and codeine with a cross-reactivity >25% (FIG. 6D).

Next, assessed was the specificity of these aptamers to the counter-targets at a concentration of 250 μM (100 μM was used for papaverine, noscapine, alprazolam, diazepam, clonazepam) relative to 100 μM target using the T5 Exo/Exo I assay. Most HM aptamers did not respond to the synthetic opioids fentanyl and acetyl fentanyl (FIG. 6C), which is imperative for the practical use of these aptamers given that seized drugs and counterfeit medications are often adulterated with such substances. HM2, HM4, HM20, HM29, HM30, and HM31 were the most specific aptamers, with cross-reactivities <10% to all tested interferents. The remaining HM aptamers had moderate to severe cross-reactivity to various counter-targets. Notably, although papaverine consistently eluted a large quantity of the pool during counter-SELEX, all aptamers had minimal cross-reactivity to this compound except for HM1 and HM31.2 (20-22% cross-reactivity). For the OM aptamers, OM3, OM4, OM9, OM14, OM16, and OM19 had high specificity against all interferents, even naloxone, with <15% cross-reactivity. Among these, OM4, OM9, OM14, OM16, and OM19 had zero cross-reactivity to naloxone, which is impressive given the high degree of structural similarity between oxycodone/oxymorphone and naloxone. Other OM aptamers had poorer specificity, particularly against naloxone (FIG. 6D).

Based on these data, three different types of aptamers were identified with a particular pattern of preference for the opioid targets. The first, which includes most HM aptamers (of which HM20 was the best performing), bound to morphine, heroin, codeine, hydrocodone, and hydromorphone with >90% cross-reactivity relative to heroin, but not to oxycodone and oxymorphone, which primarily differ by a hydroxy group at C14. The second type, of which OM9 showed the best affinity and specificity, bound primarily to oxycodone and oxymorphone with >90% cross-reactivity relative to oxycodone and exhibited less cross-reactivity to hydrocodone and hydromorphone (≤50%), which lack the hydroxy group at C14 but retain the C6 ketone. However, these aptamers did not bind morphine, heroin, or codeine, which lack both the C14 hydroxy group and the C6 ketone. The third category, of which OM4 was the best, bound oxycodone, oxymorphone, hydrocodone, and hydromorphone (i.e., the prescription opioids) with >90% cross-reactivity relative to oxycodone, but with no affinity to morphine, heroin, or codeine, which all lack the C6 ketone.

### Example 6

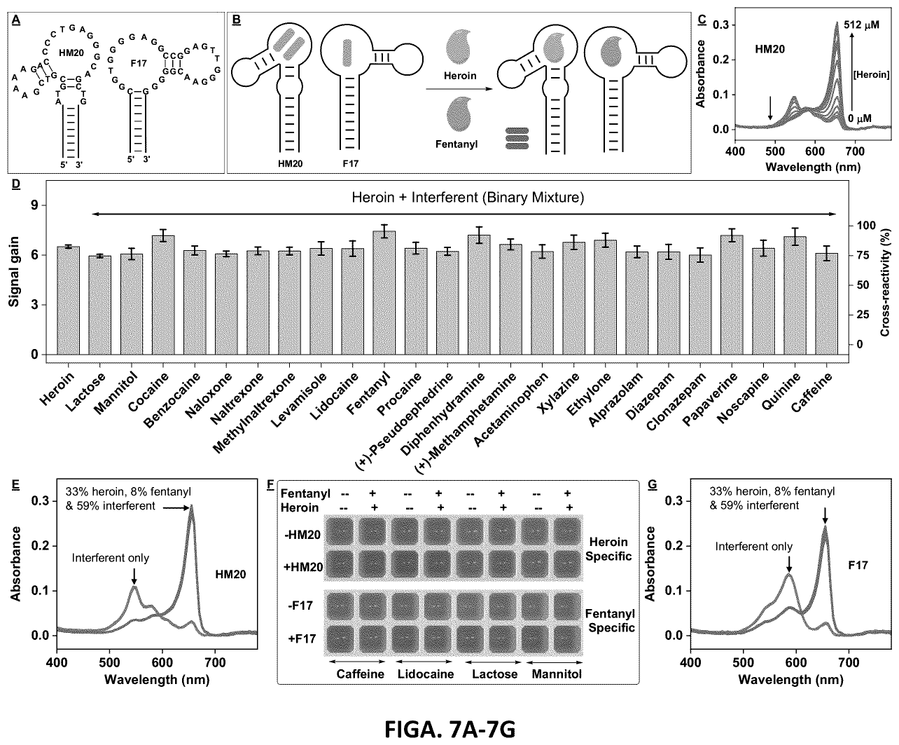

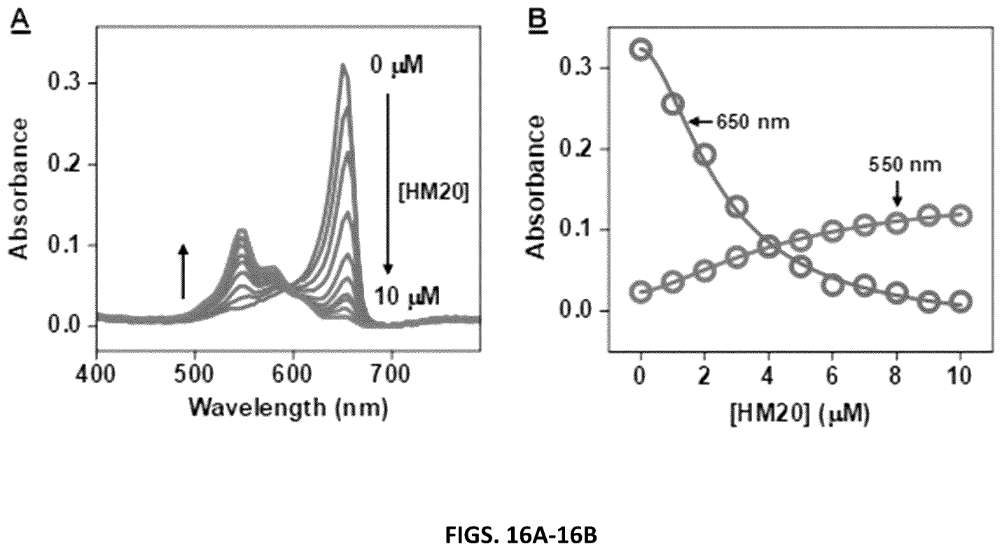

Aptamer-based dye-displacement assay for colorimetric detection of morphine-related opioids. A HM20 was used, which was the aptamer with the highest affinity and specificity from the selections, (FIG. 7A), to develop colorimetric aptamer-based dye-displacement assays for on-site detection of opioids in seized substances and counterfeit pills. Based on previous work, employed was the DNA-binding cyanine dye 3,3′-di (3-sulfopropyl)-4,5,4′,5′-dibenzo-9-methyl-thiacarbocyanine (MTC), which binds aptamers as monomers and dimers in the absence of target. When the target is added to the aptamer-dye complex, the dye is liberated into solution and forms J-aggregates, resulting in a purple-to-blue color change (FIG. 7B). To detect heroin, the concentration of HM20 was optimized and required to deplete MTC aggregates and form aptamer-dye complexes by mixing 2.5 μM MTC with various concentrations of aptamer (0-10 μM) in aqueous buffer. It was observed that there was an increase in monomer and dimer absorbance at 585 nm and 550 nm, respectively, and a decrease in J-aggregates that absorb maximally at 650 nm as a function of aptamer concentration, with nearly 90% bound to the aptamer at ˜6 μM aptamer (FIG. 16). Notably, unlike other aptamers that have been tested with MTC previously, HM20 prefers to bind MTC dimer as opposed to monomer. Since the dimer has an absorbance peak that is farther from the J-aggregate absorbance peak, this potentially increases the assay color contrast to allow for more confident identification of a color change with the naked eye. Next, the aptamer-dye complexes with varying concentrations of heroin (0-512 μM) were challenged and observed a target-concentration-dependent increase in J-aggregate absorbance and decreases in dimer and monomer absorbance (FIG. 7C). To quantify the signal change, the ratio between the AUC of the absorbance spectra between 590-680 nm (for J-aggregates) and 500-590 nm (for monomers/dimers) was calculated.

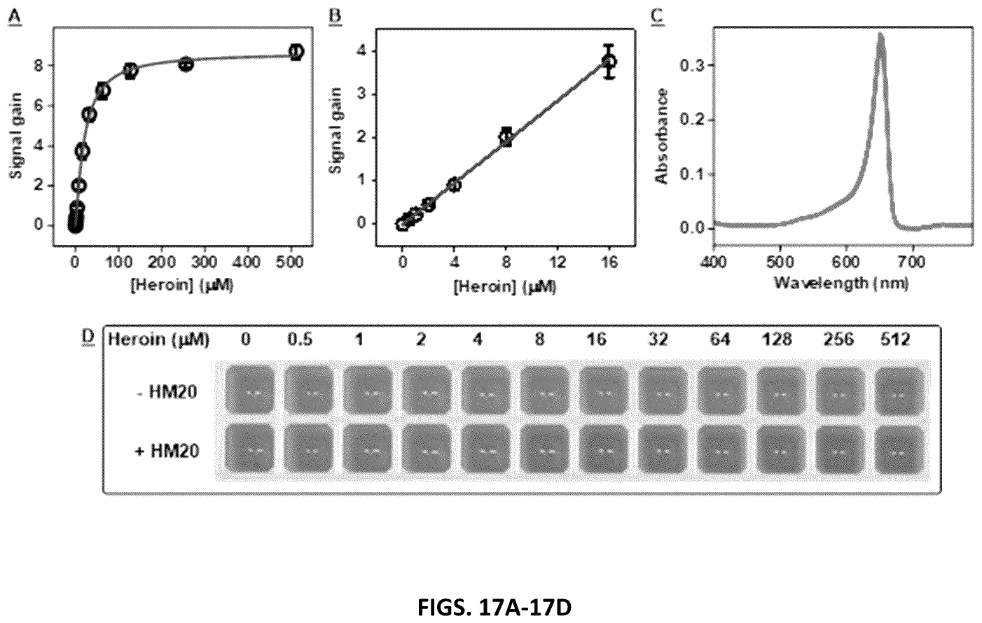

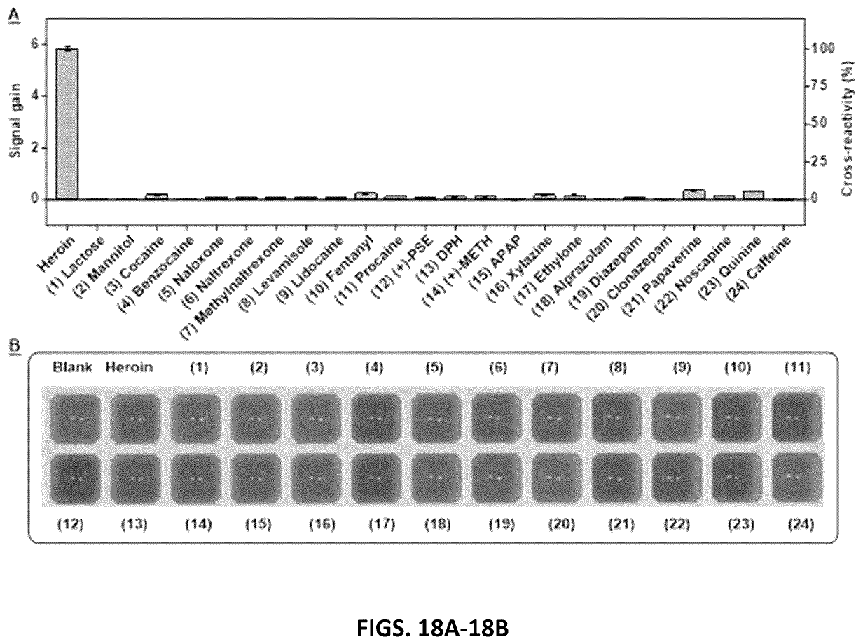

Using this ratio, the signal gains and plotted these values as a function of target concentration to construct a calibration curve were also calculated (FIG. 17A). Based on these data, the assay had an instrumental limit of detection of 0.5 μM with a linear range from 0-16 μM (FIG. 17B). A control experiment performed by mixing the dye with varying concentrations of heroin demonstrated that the target itself does not perturb dye aggregation state or absorbance spectra (FIG. 17C). Notably, the presence of heroin can be clearly identified at as low as 8 μM with the naked eye via a pink-to-blue color change (FIG. 17D), which is sufficiently sensitive for detecting opioids in seized drug samples. Next, determined was the specificity of the assay against a variety of interferents commonly found in illicit drug samples. Since the purity of heroin is often 30-50%, assessed next was the specificity of the assay by challenging aptamer-dye complexes with 25 μM heroin or 50 μM interferent. The assay did not have any meaningful cross-reactivity to any of these interferents (FIG. 18A), and only the heroin sample induced a pink-to-blue color change that was visible to the naked eye (FIG. 18B). After, the assay with binary mixtures of 25 μM heroin (˜33%) and 50 μM interferent (˜67%) was challenged, it was found that none of the interferents altered the signals produced by heroin, demonstrating the excellent specificity of the assay (FIG. 7D).

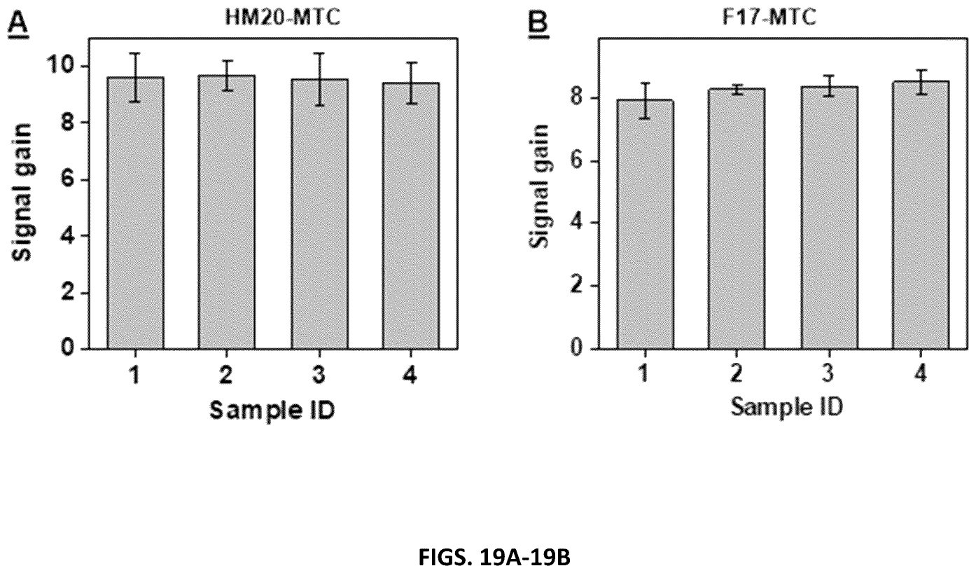

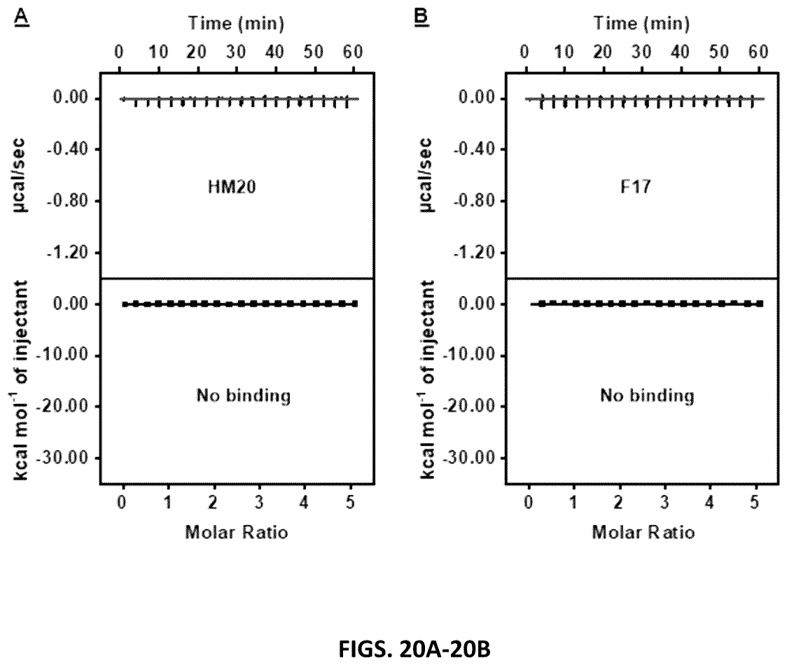

Studies have found that heroin, among other drugs, have been increasingly laced with fentanyl. To determine if this assay could specifically detect heroin in drug mixtures, heroin (33%; 25 μM) and fentanyl (8%; 6 μM) was combined with common cutting agents such as caffeine, lidocaine, lactose, or mannitol (59%; 45 μM). Next, the HM20-based dye-displacement assay, with samples, was challenged and it was found that the assay could specifically and accurately detect heroin even in the presence of cutting agents and fentanyl (FIG. 7E, F and FIG. 19A). In order to test the ability to detect heroin and fentanyl in parallel with this assay format, utilized was a recently isolated aptamer F17, which binds fentanyl with nanomolar affinity, to detect this drug in these drug mixtures. The F17-based assay could detect fentanyl via a pink-to-blue color change despite its low quantity in the mixture, without any response to heroin and other interferents. (FIG. 7F, G and FIG. 19B). These results are consistent with ITC in that HM20 does not bind to fentanyl (FIG. 20A) and F17 does not bind heroin (FIG. 20B). The aptamer-based dye-displacement assays thus enabled visual detection of heroin and fentanyl in a specific and sensitive manner, even in complex mixtures containing both drugs along with other interferents.

### Example 7

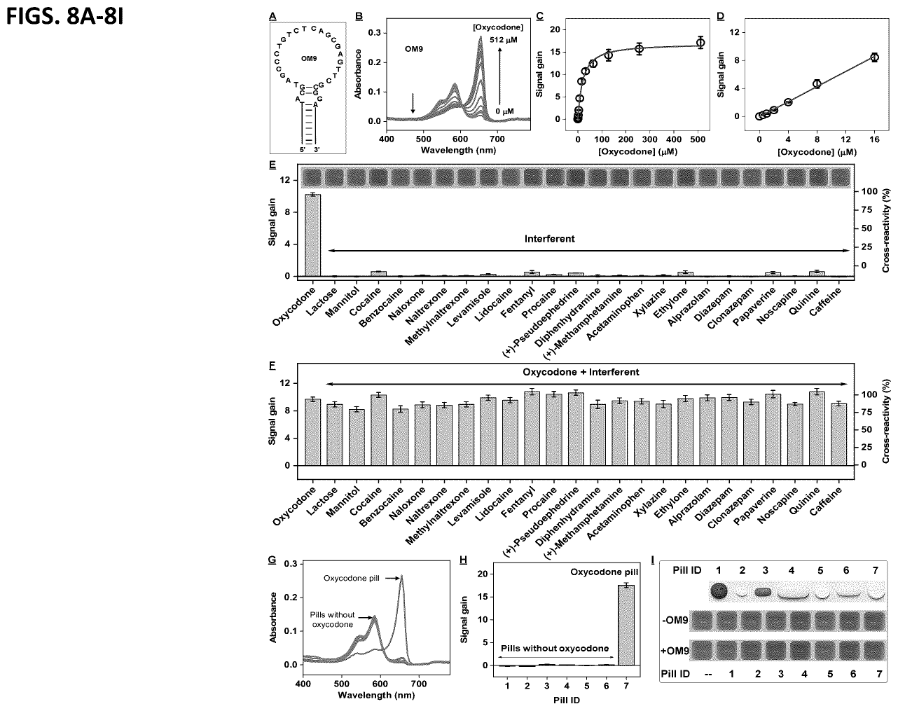

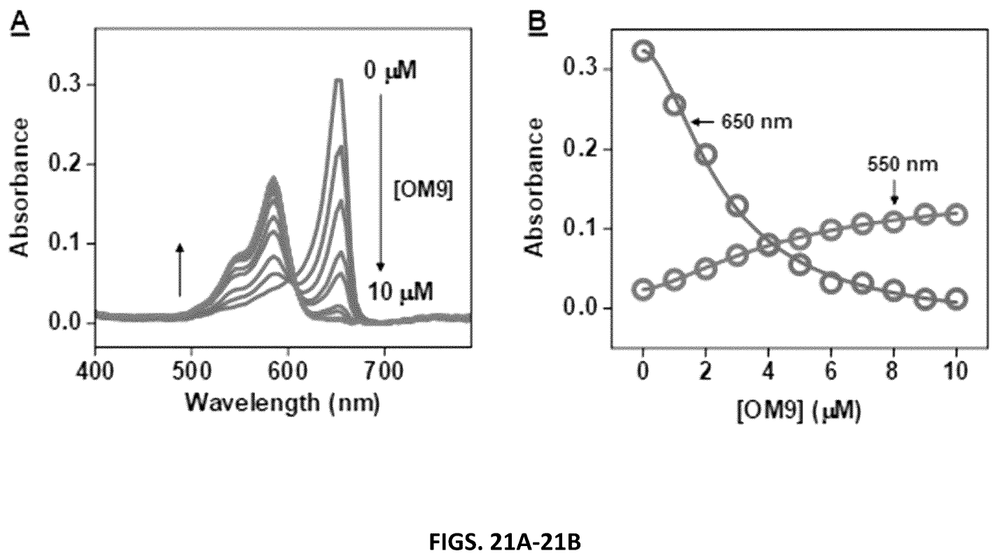

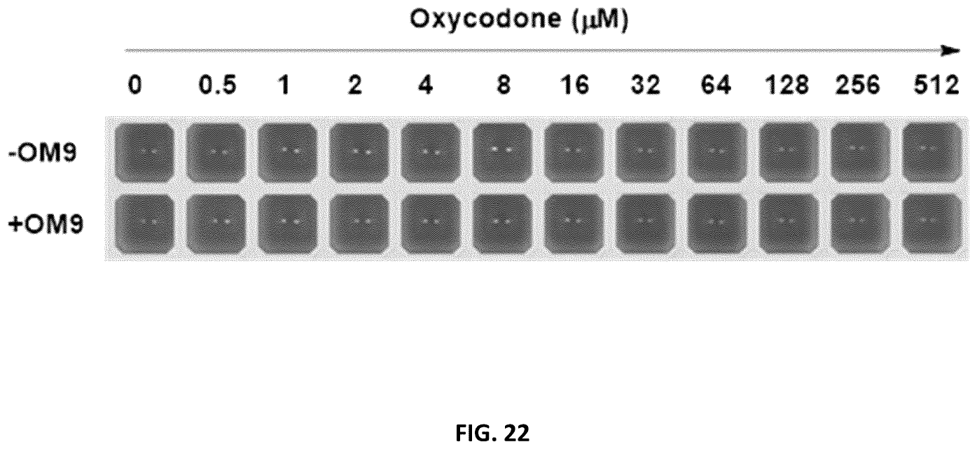

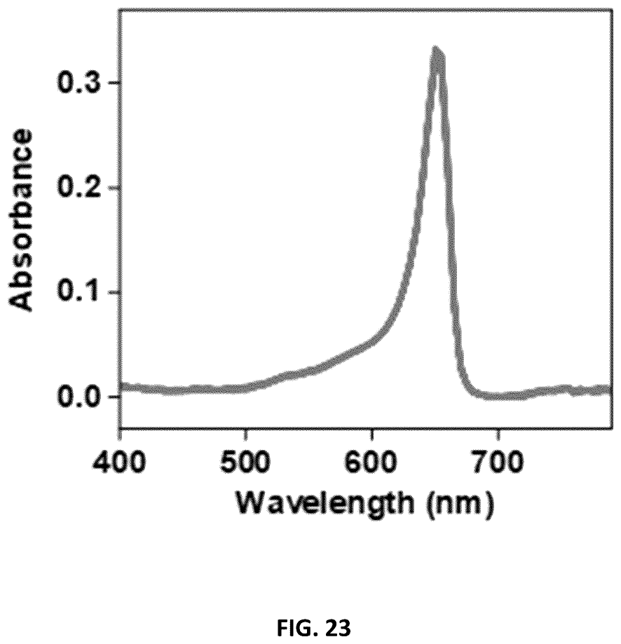

MTC-displacement assay using aptamer OM9. First, the MTC-displacement assay using aptamer OM9 (FIG. 8A) for detection of oxycodone was developed. Aptamer optimization experiments indicated that 85% of the dye was bound to the aptamer at 4 μM OM9 (FIG. 21). Next, the aptamer-dye complex with various concentrations of oxycodone (0-512 μM) was challenged (FIG. 8B) and what was observed was a color change with the naked eye at 8 μM oxycodone (FIG. 22), with an instrumental detection limit of 0.5 μM and a linear range from 0-16 μM (FIG. 8C, D). Control experiments confirmed oxycodone itself does not perturb the dye aggregation state or absorbance spectra (FIG. 23). It was also determined that the specificity of the assay was challenged with 25 μM oxycodone or 50 μM interferent. OM9 demonstrated excellent specificity, with little-to-no response to any of the interferent molecules and only oxycodone producing a visible pink-to-blue color change (FIG. 8E). Finally, the assay with binary mixtures of 25 μM oxycodone and 50 μM interferent was challenged and what was observed was identical color changes as well as signal response from oxycodone no matter whether the interferents were present or not (FIG. 8F).

Diversion and counterfeiting of prescription opioid pills containing oxycodone and hydrocodone has been a major contributor to the opioid epidemic. It was assessed whether the OM9 dye-displacement assay could accurately differentiate legitimate opioid-containing prescription medicines from other pharmaceutical tablets. Specifically, tested was the response of the assays to a 5 mg hydrocodone tablet containing 325 mg acetaminophen and a 5 mg oxycodone tablet with 325 mg acetaminophen, as well as other common over-the-counter drugs including Advil (200 mg ibuprofen), Claritin (10 mg loratadine), Benadryl (25 mg diphenhydramine), Mucinex (60 mg dextromethorphan and 1,200 mg guaifenesin), and Tylenol (325 mg acetaminophen). For drug testing, each tablet was first crushed and extracted in 10 mL of water containing 10% MeOH (v/v) for 30 mins at room temperature with intermittent shaking. Afterwards, the sample was centrifuged, and the supernatant was filtered with a 0.45 μm mixed cellulose ester filter to remove particulates. This extract was then diluted five-fold in the selection buffer, and this diluted sample was used for the dye-displacement assay. The assay yielded a positive response to the oxycodone tablets with a clear pink-to-blue color change, and little-to-no response to the other tablets (FIG. 8F, H). This was important as dextromethorphan, the active ingredient in Mucinex, has a high degree of structural similarity to oxycodone. Thus, it is evident that the aptamer-based dye displacement assays can offer an improved on-site screening test for differentiating actual opioid-containing tablets from other over the counter or prescription medicines.

1. A single-stranded nucleic acid molecule capable of specifically binding heroin or morphine, or a derivative or analog thereof, comprising a nucleic acid sequence that is at least 50% identical to the following: GGATTCG(X)₁₆₋₁₈CTCGT; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; X₁₅ is A, T, C, or G; X₁₆ is A, T, C, or G; X₁₇ is A, T, C, or G; and X₁₈ is A, T, C, or G (SEQ ID NO: 1).

2. The nucleic acid molecule of claim 1, wherein X₁ is G or C; X₂ is A or G; X₃ is T or C; X₄ is C; X₅ is G or C; X₆ is T or C; X₇ is G or A; X₈ is G or A; X₉ is A or G; X₁₀ is A or T; X₁₁ is C or G; X₁₂ is A or G; X₁₃ is G; X₁₄ is T or G; X₁₅ is G or A; X₁₆ is C or G; X₁₇ is G; and X₁₈ is G (SEQ ID NO: 2).

3. The nucleic acid molecule of claim 1, wherein the nucleic acid molecule comprises a nucleic acid sequence that is at least 50% identical to SEQ ID NOs: 9 or 10.

4-8. (canceled)

9. A single-stranded nucleic acid molecule capable of specifically binding heroin or morphine, or a derivative or analog thereof, comprising a nucleic acid sequence that is at least 50% identical to the following: TAGC(X)₃₋₄GCGTTGTTCGA(X)₆₋₈AGTA; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; and X₁₂ is A, T, C, or G (SEQ ID NO: 5).

10. The nucleic acid molecule of claim 9, wherein X₁ is C, G, or T; X₂ is C or G; X₃ is A, T, C, or G; X₄ is G, T, or A; X₅ is G, A, or C; X₆ is T, C, or G; X₇ is A, T, C, or G; X₈ is C, A, or T; X₉ is A, G, or T; X₁₀ is G, T, or C; X₁₁ is C or G; and X₁₂ is G or A (SEQ ID NO: 6).

11. The nucleic acid molecule of claim 10, wherein the nucleic acid molecule comprises a nucleic acid sequence that is at least 50% identical to any one of SEQ ID NOs: 18-26.

12-16. (canceled)

17. A single-stranded nucleic acid molecule capable of specifically binding heroin or morphine, or a derivative or analog thereof, comprising a nucleic acid sequence that is at least 50% identical to any one of SEQ ID NOs: 29-33.

18. A single-stranded nucleic acid molecule capable of specifically binding oxycodone or oxymorphone, or a derivative or analog thereof, comprising a nucleic acid sequence that is at least 50% identical to the following: (X)₁₂₋₁₃TCTCAGCGAGTTCG (X)₃₋₄; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; X₁₅ is A, T, C, or G; X₁₆ is A, T, C, or G; and X₁₇ is A, T, C, or G (SEQ ID NO: 34).

19. The nucleic acid molecule of claim 18, wherein X₁ is A, T, or G; X₂ is A, C, or G; X₃ is G, C, or A; X₄ is T or G; X₅ is C or T; X₆ is A; X₇ is G; X₈ is G, C, or T; X₉ is C or T; X₁₀ is C; X₁₁ is T or A; X₁₂ is G or T; X₁₃ is C or G; X₁₄ is A, C, or T; X₁₅ is C, G, or T; X₁₆ is A or G; and X₁₇ is T or A (SEQ ID NO: 35).

20. The nucleic acid molecule of claim 18, wherein the nucleic acid molecule comprises a nucleic acid sequence that is at least 50% identical to any one of SEQ ID NOs: 46-51.

21-25. (canceled)

26. A single-stranded nucleic acid molecule capable of specifically binding oxycodone or oxymorphone, or a derivative or analog thereof, comprising a nucleic acid sequence that is at least 50% identical to the following: (X)₂₋₆GGGAGT(X)₂₋₃GTTTGTGTGGGG(X)₂₋₆; wherein X₁ is A, T, C, or G; X₂ is A, T, C, or G; X₃ is A, T, C, or G; X₄ is A, T, C, or G; X₅ is A, T, C, or G; X₆ is A, T, C, or G; X₇ is A, T, C, or G; X₈ is A, T, C, or G; X₉ is A, T, C, or G; X₁₀ is A, T, C, or G; X₁₁ is A, T, C, or G; X₁₂ is A, T, C, or G; X₁₃ is A, T, C, or G; X₁₄ is A, T, C, or G; and X₁₅ is A, T, C, or G (SEQ ID NO: 38).

27. The nucleic acid molecule of claim 26, wherein X₁ is G; X₂ is A or G; X₃ is G; X₄ is T; X₅ is G; X₆ is C; X₇ is G, A, or T; X₈ is T or A; X₉ is T; X₁₀ is T, A, or G; X₁₁ is C or G; X₁₂ is C or T; X₁₃ is C; X₁₄ is G or C; and X₁₅ is A (SEQ ID NO: 39).

28. The nucleic acid molecule of claim 26, wherein the nucleic acid molecule comprises a nucleic acid sequence that is at least 50% identical to any one of SEQ ID NOs: 54-56.

29-41. (canceled)

42. A single-stranded nucleic acid molecule capable of specifically binding oxycodone or oxymorphone, or a derivative or analog thereof, comprising a nucleic acid sequence that is at least 50% identical to any one of SEQ ID NOs: 66-70.

43. The nucleic acid molecule of claim 1, wherein the nucleic acid molecule comprises a detection moiety.

44. The nucleic acid molecule of claim 1, wherein the nucleic acid molecule is in solution or attached to a substrate.

45. (canceled)

46. A method of detecting an opioid, or a derivative or analog thereof, the method comprising:
combining the nucleic acid molecule of claim 1 comprising a fluorescent moiety with a quencher-labeled nucleic acid molecule that is at least partially complementary to the nucleic acid molecule of claim 1 to form a quenched composition; and
exposing the quenched composition to a sample comprising or suspected of comprising an opioid, or a derivative or analog thereof;
wherein presence of the opioid, or a derivative or analog thereof, in the sample displaces the quencher-labeled nucleic acid molecule, thereby producing a fluorescent signal proportional to the concentration of the opioid, or a derivative or analog thereof, in the sample.

47. A method of detecting opioid, or a derivative or analog thereof, the method comprising:
combining the nucleic acid molecule of claim 1 with a reporter compound that binds to the nucleic acid molecule of claim 1 to form a complexed composition; and
exposing the complexed composition to a sample comprising or suspected of comprising an opioid, or a derivative or analog thereof;
wherein presence of the opioid, or a derivative or analog thereof, in the sample displaces the reporter compound, thereby allowing the reporter compound to form detectable aggregates proportional to the concentration of the opioid, or a derivative or analog thereof, in the sample.

48. A method of detecting opioid, or a derivative or analog thereof, the method comprising:
immobilizing the nucleic acid molecule of claim 1 to an electrically conductive substrate, wherein the nucleic acid molecule of claim 1 comprise a redox tag, to form a detection sensor; and
exposing the detection sensor to a sample comprising or suspected of comprising an opioid, or a derivative or analog thereof;
wherein presence of the opioid, or a derivative or analog thereof, in the sample binds the nucleic acid molecule of claim 1, thereby producing an electrochemical signal proportional to the concentration of the opioid, or a derivative or analog thereof, in the sample.

49. The method of claim 46, wherein the sample is a biological sample from a human subject, and wherein the biological sample is a saliva sample, a urine sample, a blood sample, a serum sample, a plasma sample, a fecal sample, a CSF sample, or a tissue sample.

50. (canceled)


SEQUENCE LISTING (126 sequences)

SEQ ID NO: 1 - DNA, 30 bp, Synthetic construct
  source 1..30: mol_type = other DNA
  variation 8..25: note = N is A, T, C, or G
ggattcgnnnnnnnnnnnnnnnnnnctcgt

SEQ ID NO: 2 - DNA, 30 bp, Synthetic construct; identical to SEQ ID NO: 1
  source 1..30: mol_type = other DNA
  variation 8: note = N is G or C
  variation 9: note = N is A or G
  variation 10: note = N is T or C
  variation 11: note = N is C
  variation 12: note = N is G or C
  variation 13: note = N is T or C
  variation 14: note = N is G or A
  variation 15: note = N is G or A
  variation 16: note = N is A or G
  variation 17: note = N is A or T
  variation 18: note = N is C or G
  variation 19: note = N is A or G
  variation 20: note = N is G
  variation 21: note = N is T or G
  variation 22: note = N is G or A
  variation 23: note = N is C or G
  variation 24..25: note = N is G

SEQ ID NO: 3 - DNA, 30 bp, Synthetic construct
  source 1..30: mol_type = other DNA
  variation 6..12: note = N is A, T, C, or G
  variation 16..20: note = N is A, T, C, or G
gcgtannnnnnntagnnnnncgtcgttcaa

SEQ ID NO: 4 - DNA, 30 bp, Synthetic construct; identical to SEQ ID NO: 3
  source 1..30: mol_type = other DNA
  variation 6: note = N is G or C
  variation 7: note = N is G, C, or T
  variation 8: note = N is T or A
  variation 9: note = N is T, C, or G
  variation 10: note = N is T, C, or G
  variation 11: note = N is C, G, or A
  variation 12: note = N is C or G
  variation 16: note = N is C or T
  variation 17: note = N is T or C
  variation 18: note = N is G
  variation 19: note = N is T or C
  variation 20: note = N is G or A

SEQ ID NO: 5 - DNA, 31 bp, Synthetic construct
  source 1..31: mol_type = other DNA
  variation 5..8: note = N is A, T, C, or G
  variation 20..27: note = N is A, T, C, or G
tagcnnnngcgttgttcgannnnnnnnagta

SEQ ID NO: 6 - DNA, 31 bp, Synthetic construct; identical to SEQ ID NO: 5
  source 1..31: mol_type = other DNA
  variation 5: note = N is C, G, or T
  variation 6: note = N is C or G
  variation 7: note = N is A, T, C, or G
  variation 8: note = N is G, T, or A
  variation 20: note = N is G, A, or C
  variation 21: note = N is T, C, or G
  variation 22: note = N is A, T, C, or G
  variation 23: note = N is C, A, or T
  variation 24: note = N is A, G, or T
  variation 25: note = N is G, T, or C
  variation 26: note = N is C or G
  variation 27: note = N is G or A

SEQ ID NO: 7 - DNA, 30 bp, Synthetic construct
  source 1..30: mol_type = other DNA
  variation 12..19: note = N is A, T, C, or G
agggcacgtctnnnnnnnnagggtttcgcg

SEQ ID NO: 8 - DNA, 30 bp, Synthetic construct; identical to SEQ ID NO: 7
  source 1..30: mol_type = other DNA
  variation 12: note = N is C or G
  variation 13: note = N is G or T
  variation 14: note = N is G
  variation 16: note = N is A or G
  variation 15: note = N is C or T
  variation 17: note = N is T or C
  variation 18: note = N is C
  variation 19: note = N is G

SEQ ID NO: 9 - DNA, 30 bp, Synthetic construct
  source 1..30: mol_type = other DNA
gggattcggatcgtggaacagtgcctcgtc

SEQ ID NO: 10 - DNA, 31 bp, Synthetic construct
  source 1..31: mol_type = other DNA
gggattcgcgccccaagtggggagggctcgt

SEQ ID NO: 11 - DNA, 30 bp, Synthetic construct
  source 1..30: mol_type = other DNA
gcgtaggtttcctagctgtgcgtcgttcaa

SEQ ID NO: 12 - DNA, 30 bp, Synthetic construct
  source 1..30: mol_type = other DNA
gcgtaggtctcctagctgtgcgtcgttcaa

SEQ ID NO: 13 - DNA, 30 bp, Synthetic construct
  source 1..30: mol_type = other DNA
gcgtaggttccctagctgtgcgtcgttcaa

SEQ ID NO: 14 - DNA, 30 bp, Synthetic construct
  source 1..30: mol_type = other DNA
gcgtacctgtggtagttgtacgtcgttcaa

SEQ ID NO: 15 - DNA, 30 bp, Synthetic construct
  source 1..30: mol_type = other DNA
gcgtacttccagtagccgtgcgtcgttcaa

SEQ ID NO: 16 - DNA, 30 bp, Synthetic construct
  source 1..30: mol_type = other DNA
gcgtaccatgggtagttgcacgtcgtccaa

SEQ ID NO: 17 - DNA, 30 bp, Synthetic construct
  source 1..30: mol_type = other DNA
gcgtacctgtggtagtcgtacgtcgttcaa

SEQ ID NO: 18 - DNA, 30 bp, Synthetic construct
  source 1..30: mol_type = other DNA
tagcccggcgttgttcgagtgcagcgagta

SEQ ID NO: 19 - DNA, 30 bp, Synthetic construct
  source 1..30: mol_type = other DNA
tagccgtggcgttgtccgaaccagtgagta

SEQ ID NO: 20 - DNA, 30 bp, Synthetic construct
  source 1..30: mol_type = other DNA
tagccgcgcgttgtccgacccttggaagta

SEQ ID NO: 21 - DNA, 30 bp, Synthetic construct
  source 1..30: mol_type = other DNA
tagcgcatgcgttgttcgaggatacgagta

SEQ ID NO: 22 - DNA, 30 bp, Synthetic construct
  source 1..30: mol_type = other DNA
tagctggtgcgttgtccgaagtcatgagta

SEQ ID NO: 23 - DNA, 30 bp, Synthetic construct
  source 1..30: mol_type = other DNA
tagccctagcgttgttcgagtccatgagta

SEQ ID NO: 24 - DNA, 31 bp, Synthetic construct
  source 1..31: mol_type = other DNA
tagccgtggcgttgttcgagtgcagcgagta

SEQ ID NO: 25 - DNA, 31 bp, Synthetic construct
  source 1..31: mol_type = other DNA
aagtaccaagggtagctctgcgttgttcgag

SEQ ID NO: 26 - DNA, 30 bp, Synthetic construct
  source 1..30: mol_type = other DNA
cgttgttcgagccttgagtaccattggtag

SEQ ID NO: 27 - DNA, 30 bp, Synthetic construct
  source 1..30: mol_type = other DNA
agggcacgtctcggcatcgagggtttcgcg

SEQ ID NO: 28 - DNA, 30 bp, Synthetic construct
  source 1..30: mol_type = other DNA
gagggcacgtctgtgtgcagggtttcgcgc

SEQ ID NO: 29 - DNA, 30 bp, Synthetic construct
  source 1..30: mol_type = other DNA
gggaaggttggcagcgcatcgtcgggtcac

SEQ ID NO: 30 - DNA, 30 bp, Synthetic construct
  source 1..30: mol_type = other DNA
cgttggcgagtgtgtccggggactacaggg

SEQ ID NO: 31 - DNA, 30 bp, Synthetic construct
  source 1..30: mol_type = other DNA
atgcgtcgaaaagacccctgagggcagctg

SEQ ID NO: 32 - DNA, 30 bp, Synthetic construct
  source 1..30: mol_type = other DNA
ggggctgtgggagggggttcatgggtcccg

SEQ ID NO: 33 - DNA, 30 bp, Synthetic construct
  source 1..30: mol_type = other DNA
atgggatcatgcctagtaatgatgtggtgt

SEQ ID NO: 34 - DNA, 31 bp, Synthetic construct
  source 1..31: mol_type = other DNA
  variation 1..13: note = N is A, T, C, or G
  variation 28..31: note = N is A, T, C, or G
nnnnnnnnnnnnntctcagcgagttcgnnnn

SEQ ID NO: 35 - DNA, 31 bp, Synthetic construct; identical to SEQ ID NO: 34
  source 1..31: mol_type = other DNA
  variation 1: note = N is A, T, or G
  variation 2: note = N is A, C, or G
  variation 3: note = N is G, C, or A
  variation 4: note = N is T or G
  variation 5: note = N is C or T
  variation 6: note = N is A
  variation 7: note = N is G
  variation 8: note = N is G, C, or T
  variation 9: note = N is C or T
  variation 10: map = N is C
  variation 11: note = N is T or A
  variation 12: note = N is G or T
  variation 13: note = N is C or G
  variation 28: note = N is A, C, or T
  variation 29: note = N is C, G, or T
  variation 30: note = N is A or G
  variation 31: note = N is T or A

SEQ ID NO: 36 - DNA, 30 bp, Synthetic construct
  source 1..30: mol_type = other DNA
  variation 5..15: note = N is A, T, C, or G
  variation 24: note = N is A, T, C, or G
ggctnnnnnnnnnnngtaggggtncacgct

SEQ ID NO: 37 - DNA, 30 bp, Synthetic construct; identical to SEQ ID NO: 36
  source 1..30: mol_type = other DNA
  variation 5: note = N is T
  variation 6: note = N is A or G
  variation 7: note = N is G or C
  variation 8: note = N is C or A
  variation 9..10: note = N is A or T
  variation 11: note = N is A or G
  variation 12: note = N is G or A
  variation 13: note = N is C or G
  variation 14: note = N is T or C
  variation 15: note = N is A
  variation 24: note = N is C or A

SEQ ID NO: 38 - DNA, 33 bp, Synthetic construct
  source 1..33: mol_type = other DNA
  variation 1..6: note = N is A, T, C, or G
  variation 13..15: note = N is A, T, C, or G
  variation 28..33: note = N is A, T, C, or G
nnnnnngggagtnnngtttgtgtggggnnnnnn

SEQ ID NO: 39 - DNA, 33 bp, Synthetic construct; identical to SEQ ID NO: 38
  source 1..33: mol_type = other DNA
  variation 1: note = N is G
  variation 2: note = N is A or G
  variation 3: note = N is G
  variation 4: note = N is T
  variation 5: note = N is G
  variation 6: note = N is C
  variation 13: note = N is G, A, or T
  variation 14: note = N is T or A
  variation 15: note = N is T
  variation 28: note = N is T, A, or G
  variation 29: note = N is C or G
  variation 30: note = N is C or T
  variation 31: note = N is C
  variation 32: note = N is G or C
  variation 33: note = N is A

SEQ ID NO: 40 - DNA, 32 bp, Synthetic construct
  source 1..32: mol_type = other DNA
  variation 1..2: note = N is A, T, C, or G
  variation 11: note = N is A, T, C, or G
  variation 19..24: note = N is A, T, C, or G
  variation 31..32: note = N is A, T, C, or G
nncgtaggggnacacgctnnnnnngggctgnn

SEQ ID NO: 41 - DNA, 32 bp, Synthetic construct; identical to SEQ ID NO: 40
  source 1..32: mol_type = other DNA
  variation 1: note = N is C or A
  variation 2: note = N is T or G
  variation 11: note = N is T, C, or A
  variation 19: note = N is C or T
  variation 20: note = N is T, A, or G
  variation 21: note = N is A or C
  variation 22: note = N is T, C, or A
  variation 23: note = N is A or G
  variation 24: note = N is G
  variation 31: note = N is G or A
  variation 32: note = N is T or G

SEQ ID NO: 42 - DNA, 30 bp, Synthetic construct
  source 1..30: mol_type = other DNA
  variation 8..21: note = N is A, T, C, or G
atgggatnnnnnnnnnnnnnnatgtggtgt

SEQ ID NO: 43 - DNA, 30 bp, Synthetic construct; identical to SEQ ID NO: 42
  source 1..30: mol_type = other DNA
  variation 8: note = N is A or G
  variation 9: note = N is C or G
  variation 10: note = N is G, A, or C
  variation 11: note = N is A or G
  variation 12: note = N is A, C, or G
  variation 13: note = N is C, T, or A
  variation 14: note = N is T, A, or G
  variation 15: note = N is C or T
  variation 16: note = N is G or C
  variation 17: note = N is T or C
  variation 18: note = N is T or G
  variation 19: note = N is T, A, or G
  variation 20: note = N is G
  variation 21: note = N is G or C

SEQ ID NO: 44 - DNA, 31 bp, Synthetic construct
  source 1..31: mol_type = other DNA
  variation 1..4: note = N is A, T, C, or G
  variation 14..21: note = N is A, T, C, or G
  variation 29..31: note = N is A, T, C, or G
nnnnatgtggtgtnnnnnnnnatgggatnnn

SEQ ID NO: 45 - DNA, 31 bp, Synthetic construct; identical to SEQ ID NO: 44
  source 1..31: mol_type = other DNA
  variation 1: note = N is G
  variation 2: note = N is C or G
  variation 3: note = N is G
  variation 4: note = N is C
  variation 14: note = N is C
  variation 15: note = N is G or A
  variation 16: note = N is G
  variation 17: note = N is T
  variation 18: note = N is C
  variation 19: note = N is A
  variation 20: note = N is G or T
  variation 21: note = N is G
  variation 29: note = N is G or C
  variation 30..31: note = N is A

SEQ ID NO: 46 - DNA, 30 bp, Synthetic construct
  source 1..30: mol_type = other DNA
aagtcaggcctgtctcagcgagttcgacat

SEQ ID NO: 47 - DNA, 30 bp, Synthetic construct
  source 1..30: mol_type = other DNA
aagtcaggtctgtctcagcgagttcgacat

SEQ ID NO: 48 - DNA, 30 bp, Synthetic construct
  source 1..30: mol_type = other DNA
tacgtagccctgtctcagcgagttcgcgga

SEQ ID NO: 49 - DNA, 30 bp, Synthetic construct
  source 1..30: mol_type = other DNA
agagtaggcctgtctcagcgagttcgctat

SEQ ID NO: 50 - DNA, 30 bp, Synthetic construct
  source 1..30: mol_type = other DNA
gcagcagtccatctctcagcgagttcgtca

SEQ ID NO: 51 - DNA, 30 bp, Synthetic construct
  source 1..30: mol_type = other DNA
tgagtaggcctgtctcagcgagttcactat

SEQ ID NO: 52 - DNA, 30 bp, Synthetic construct
  source 1..30: mol_type = other DNA
ggcttagcaaagctagtaggggtccacgct

SEQ ID NO: 53 - DNA, 30 bp, Synthetic construct
  source 1..30: mol_type = other DNA
ggcttgcattgagcagaaggggtacacgct

SEQ ID NO: 54 - DNA, 30 bp, Synthetic construct
  source 1..30: mol_type = other DNA
gagtgcggggagtgtgtttctgtgggggtc

SEQ ID NO: 55 - DNA, 30 bp, Synthetic construct
  source 1..30: mol_type = other DNA
ggggggagtaatgtttgtgtgggggagccg

SEQ ID NO: 56 - DNA, 30 bp, Synthetic construct
  source 1..30: mol_type = other DNA
ggggggagtttgtttgtgtgggagggtcca

SEQ ID NO: 57 - DNA, 30 bp, Synthetic construct
  source 1..30: mol_type = other DNA
ccgtaggggtacacgctctataggggctgg

SEQ ID NO: 58 - DNA, 30 bp, Synthetic construct
  source 1..30: mol_type = other DNA
ccgtaggggtacacgctctatgggggctgg

SEQ ID NO: 59 - DNA, 30 bp, Synthetic construct
  source 1..30: mol_type = other DNA
atcgtaggggcacacgcttaacgggctgat

SEQ ID NO: 60 - DNA, 30 bp, Synthetic construct
  source 1..30: mol_type = other DNA
cgcgtaggggaacacgcttgcagggctgag

SEQ ID NO: 61 - DNA, 30 bp, Synthetic construct
  source 1..30: mol_type = other DNA
atgggatacgaactcgtttggatgtggtgt

SEQ ID NO: 62 - DNA, 30 bp, Synthetic construct
  source 1..30: mol_type = other DNA
atgggatggagctatgcgagcatgtggtgt

SEQ ID NO: 63 - DNA, 30 bp, Synthetic construct
  source 1..30: mol_type = other DNA
atgggataccagagtcctgggatgtggtgt

SEQ ID NO: 64 - DNA, 30 bp, Synthetic construct
  source 1..30: mol_type = other DNA
gcgcatgtggtgtcggtcagatgggatgaa

SEQ ID NO: 65 - DNA, 30 bp, Synthetic construct
  source 1..30: mol_type = other DNA
gggatgtggtgtcagtcatgatgggatcaa

SEQ ID NO: 66 - DNA, 30 bp, Synthetic construct
  source 1..30: mol_type = other DNA
tggcgccggattccctaaggtacacggact

SEQ ID NO: 67 - DNA, 30 bp, Synthetic construct
  source 1..30: mol_type = other DNA
ggattgcgcttgctaacgccgtgtagagtc

SEQ ID NO: 68 - DNA, 30 bp, Synthetic construct
  source 1..30: mol_type = other DNA
cggacttgggtgggtgctggggcttgggcg

SEQ ID NO: 69 - DNA, 30 bp, Synthetic construct
  source 1..30: mol_type = other DNA
ggccatgggattgtctaatgtggtgtggaa

SEQ ID NO: 70 - DNA, 30 bp, Synthetic construct
  source 1..30: mol_type = other DNA
gcgtgggataccttttgggatgtggtgcac

SEQ ID NO: 71 - DNA, 73 bp, Synthetic construct
  source 1..73: mol_type = other DNA
ggaggctctcgggacgacnnnnnnnnnnnnnnnnnnnnnnnnnnnnnngtcgtcccgcctttaggatttacag

SEQ ID NO: 72 - DNA, 18 bp, Synthetic construct
  source 1..18: mol_type = other DNA
gtcgtcccgagagccata

SEQ ID NO: 73 - DNA, 18 bp, Synthetic construct
  source 1..18: mol_type = other DNA
ggaggctctcgggacgac

SEQ ID NO: 74 - DNA, 25 bp, Synthetic construct
  source 1..25: mol_type = other DNA
ctgtaaatcctaaaggcgggacgac

SEQ ID NO: 75 - DNA, 96 bp, Synthetic construct
  source 1..96: mol_type = other DNA
acactctttccctacacgacgctcttccgatctnnnnnnnnnnnnnnnnnnnnnnnnnnnnnnnnnnnnnnnnnnnnnggaggctctcgggacgac

SEQ ID NO: 76 - DNA, 100 bp, Synthetic construct
  source 1..100: mol_type = other DNA
gactggagttcagacgtgtgctcttccgatctnnnnnnnnnnnnnnnnnnnnnnnnnnnnnnnnnnnnnnnnnnnctgtaaatcctaaaggcgggacgac

SEQ ID NO: 77 - DNA, 46 bp, Synthetic construct
  source 1..46: mol_type = other DNA
gggacgacgggattcggatcgtggaacagtgcctcgtcgtcgtccc

SEQ ID NO: 78 - DNA, 46 bp, Synthetic construct
  source 1..46: mol_type = other DNA
gggacgacgcgtaggtttcctagctgtgcgtcgttcaagtcgtccc

SEQ ID NO: 79 - DNA, 46 bp, Synthetic construct
  source 1..46: mol_type = other DNA
gggacgactagcccggcgttgttcgagtgcagcgagtagtcgtccc

SEQ ID NO: 80 - DNA, 46 bp, Synthetic construct
  source 1..46: mol_type = other DNA
gggacgactagccgtggcgttgtccgaaccagtgagtagtcgtccc

SEQ ID NO: 81 - DNA, 46 bp, Synthetic construct
  source 1..46: mol_type = other DNA
gggacgacagggcacgtctcggcatcgagggtttcgcggtcgtccc

SEQ ID NO: 82 - DNA, 46 bp, Synthetic construct
  source 1..46: mol_type = other DNA
gggacgacgggaaggttggcagcgcatcgtcgggtcacgtcgtccc

SEQ ID NO: 83 - DNA, 46 bp, Synthetic construct
  source 1..46: mol_type = other DNA
gggacgacgagggcacgtctgtgtgcagggtttcgcgcgtcgtccc

SEQ ID NO: 84 - DNA, 46 bp, Synthetic construct
  source 1..46: mol_type = other DNA
gggacgacgcgtaggtctcctagctgtgcgtcgttcaagtcgtccc

SEQ ID NO: 85 - DNA, 46 bp, Synthetic construct
  source 1..46: mol_type = other DNA
gggacgacgcgtacctgtggtagttgtacgtcgttcaagtcgtccc

SEQ ID NO: 86 - DNA, 46 bp, Synthetic construct
  source 1..46: mol_type = other DNA
gggacgacgcgtaggttccctagctgtgcgtcgttcaagtcgtccc

SEQ ID NO: 87 - DNA, 46 bp, Synthetic construct
  source 1..46: mol_type = other DNA
gggacgaccgttggcgagtgtgtccggggactacaggggtcgtccc

SEQ ID NO: 88 - DNA, 46 bp, Synthetic construct
  source 1..46: mol_type = other DNA
gggacgacatgcgtcgaaaagacccctgagggcagctggtcgtccc

SEQ ID NO: 89 - DNA, 46 bp, Synthetic construct
  source 1..46: mol_type = other DNA
gggacgacgcgtacttccagtagccgtgcgtcgttcaagtcgtccc

SEQ ID NO: 90 - DNA, 46 bp, Synthetic construct
  source 1..46: mol_type = other DNA
gggacgactagccgcgcgttgtccgacccttggaagtagtcgtccc

SEQ ID NO: 91 - DNA, 47 bp, Synthetic construct
  source 1..47: mol_type = other DNA
gggacgacaagtaccaagggtagctctgcgttgttcgaggtcgtccc

SEQ ID NO: 92 - DNA, 46 bp, Synthetic construct
  source 1..46: mol_type = other DNA
gggacgacggggctgtgggagggggttcatgggtcccggtcgtccc

SEQ ID NO: 93 - DNA, 46 bp, Synthetic construct
  source 1..46: mol_type = other DNA
gggacgactagcgcatgcgttgttcgaggatacgagtagtcgtccc

SEQ ID NO: 94 - DNA, 46 bp, Synthetic construct
  source 1..46: mol_type = other DNA
gggacgaccgttgttcgagccttgagtaccattggtaggtcgtccc

SEQ ID NO: 95 - DNA, 46 bp, Synthetic construct
  source 1..46: mol_type = other DNA
gggacgacgcgtaccatgggtagttgcacgtcgtccaagtcgtccc

SEQ ID NO: 96 - DNA, 46 bp, Synthetic construct
  source 1..46: mol_type = other DNA
gggacgactagctggtgcgttgtccgaagtcatgagtagtcgtccc

SEQ ID NO: 97 - DNA, 46 bp, Synthetic construct
  source 1..46: mol_type = other DNA
gggacgactagccctagcgttgttcgagtccatgagtagtcgtccc

SEQ ID NO: 98 - DNA, 47 bp, Synthetic construct
  source 1..47: mol_type = other DNA
gggacgacgggattcgcgccccaagtggggagggctcgtgtcgtccc

SEQ ID NO: 99 - DNA, 47 bp, Synthetic construct
  source 1..47: mol_type = other DNA
gggacgactagccgtggcgttgttcgagtgcagcgagtagtcgtccc

SEQ ID NO: 100 - DNA, 46 bp, Synthetic construct
  source 1..46: mol_type = other DNA
gggacgacatgggatcatgcctagtaatgatgtggtgtgtcgtccc

SEQ ID NO: 101 - DNA, 46 bp, Synthetic construct
  source 1..46: mol_type = other DNA
gggacgacgcgtacctgtggtagtcgtacgtcgttcaagtcgtccc

SEQ ID NO: 102 - DNA, 46 bp, Synthetic construct
  source 1..46: mol_type = other DNA
gggacgacaagtcaggcctgtctcagcgagttcgacatgtcgtccc

SEQ ID NO: 103 - DNA, 46 bp, Synthetic construct
  source 1..46: mol_type = other DNA
gggacgacggcttagcaaagctagtaggggtccacgctgtcgtccc

SEQ ID NO: 104 - DNA, 46 bp, Synthetic construct
  source 1..46: mol_type = other DNA
gggacgactggcgccggattccctaaggtacacggactgtcgtccc

SEQ ID NO: 105 - DNA, 46 bp, Synthetic construct
  source 1..46: mol_type = other DNA
gggacgacgagtgcggggagtgtgtttctgtgggggtcgtcgtccc

SEQ ID NO: 106 - DNA, 46 bp, Synthetic construct
  source 1..46: mol_type = other DNA
gggacgacccgtaggggtacacgctctataggggctgggtcgtccc

SEQ ID NO: 107 - DNA, 46 bp, Synthetic construct
  source 1..46: mol_type = other DNA
gggacgacccgtaggggtacacgctctatgggggctgggtcgtccc

SEQ ID NO: 108 - DNA, 46 bp, Synthetic construct
  source 1..46: mol_type = other DNA
gggacgacatcgtaggggcacacgcttaacgggctgatgtcgtccc

SEQ ID NO: 109 - DNA, 46 bp, Synthetic construct
  source 1..46: mol_type = other DNA
gggacgacgcagcagtccatctctcagcgagttcgtcagtcgtccc

SEQ ID NO: 110 - DNA, 46 bp, Synthetic construct
  source 1..46: mol_type = other DNA
gggacgactacgtagccctgtctcagcgagttcgcggagtcgtccc

SEQ ID NO: 111 - DNA, 46 bp, Synthetic construct
  source 1..46: mol_type = other DNA
gggacgacagagtaggcctgtctcagcgagttcgctatgtcgtccc

SEQ ID NO: 112 - DNA, 46 bp, Synthetic construct
  source 1..46: mol_type = other DNA
gggacgacggcttgcattgagcagaaggggtacacgctgtcgtccc

SEQ ID NO: 113 - DNA, 46 bp, Synthetic construct
  source 1..46: mol_type = other DNA
gggacgacatgggatacgaactcgtttggatgtggtgtgtcgtccc

SEQ ID NO: 114 - DNA, 46 bp, Synthetic construct
  source 1..46: mol_type = other DNA
gggacgacgcgcatgtggtgtcggtcagatgggatgaagtcgtccc

SEQ ID NO: 115 - DNA, 46 bp, Synthetic construct
  source 1..46: mol_type = other DNA
gggacgacggattgcgcttgctaacgccgtgtagagtcgtcgtccc

SEQ ID NO: 116 - DNA, 46 bp, Synthetic construct
  source 1..46: mol_type = other DNA
gggacgaccggacttgggtgggtgctggggcttgggcggtcgtccc

SEQ ID NO: 117 - DNA, 46 bp, Synthetic construct
  source 1..46: mol_type = other DNA
gggacgacggggggagtaatgtttgtgtgggggagccggtcgtccc

SEQ ID NO: 118 - DNA, 46 bp, Synthetic construct
  source 1..46: mol_type = other DNA
gggacgacgggatgtggtgtcagtcatgatgggatcaagtcgtccc

SEQ ID NO: 119 - DNA, 46 bp, Synthetic construct
  source 1..46: mol_type = other DNA
gggacgaccgcgtaggggaacacgcttgcagggctgaggtcgtccc

SEQ ID NO: 120 - DNA, 46 bp, Synthetic construct
  source 1..46: mol_type = other DNA
gggacgacaagtcaggtctgtctcagcgagttcgacatgtcgtccc

SEQ ID NO: 121 - DNA, 46 bp, Synthetic construct
  source 1..46: mol_type = other DNA
gggacgacggggggagtttgtttgtgtgggagggtccagtcgtccc

SEQ ID NO: 122 - DNA, 46 bp, Synthetic construct
  source 1..46: mol_type = other DNA
gggacgacggccatgggattgtctaatgtggtgtggaagtcgtccc

SEQ ID NO: 123 - DNA, 46 bp, Synthetic construct
  source 1..46: mol_type = other DNA
gggacgacgcgtgggataccttttgggatgtggtgcacgtcgtccc

SEQ ID NO: 124 - DNA, 46 bp, Synthetic construct
  source 1..46: mol_type = other DNA
gggacgactgagtaggcctgtctcagcgagttcactatgtcgtccc

SEQ ID NO: 125 - DNA, 46 bp, Synthetic construct
  source 1..46: mol_type = other DNA
gggacgacatgggatggagctatgcgagcatgtggtgtgtcgtccc

SEQ ID NO: 126 - DNA, 46 bp, Synthetic construct
  source 1..46: mol_type = other DNA
gggacgacatgggataccagagtcctgggatgtggtgtgtcgtccc